(19) United States
(12) Patent Application Publication
(10) Pub. No.: US 2026/0279575 A1
(43) Pub. Date: Sep. 17, 2026

(54) COMPUTATIONAL MEDICAL GUIDELINES AND CONNECTED KNOWLEDGE GRAPH FOR DIAGNOSIS AND TREATMENT

(71) Applicant: Evidium, Inc., San Francisco, CA (US)

(72) Inventors: Arnav SAXENA, San Francisco, CA (US); Wian STIPP, San Francisco, CA (US); Matthew MCSORLEY, Pittsburgh, PA (US); Fady NAKHLA, San Francisco, CA (US); Jennifer May LEE, London (GB); Ashwini MATHAI, San Francisco, CA (US); Sean THOMAS, San Francisco, CA (US); Carl BATE, San Francisco, CA (US)

(21) Appl. No.: 19/566,611

(22) Filed: Mar. 13, 2026

Related U.S. Application Data
(60) Provisional application No. 63/773,042, filed on Mar. 17, 2025.
(60) Provisional application No. 63/826,777, filed on Jun. 19, 2025.

(51) Int. Cl. G16H 50/20; G16H 10/60; G16H 50/70

(52) U.S. Cl. CPC G16H 50/20; G16H 10/60; G16H 50/70

(57) ABSTRACT

A computational platform includes a clinical knowledge graph having medical condition nodes to which are linked factor nodes and investigation nodes. Medical guideline nodes linked to a medical condition represent a computational guideline. Observation nodes and laboratory test nodes within the guideline link to factor nodes or investigation nodes. Therapy ingredient nodes represent drugs with a guideline. A patient is diagnosed or treated by traversing the guideline nodes associated with a particular condition. Probabilities annotated upon edges facilitate predicting a future patient state. Use cases include queries from a clinician, an insurance claims database, or an LLM, generation of FHIR value sets, and generation of executable CQL for a medical guideline. The knowledge graph receives a medical query, executes it and outputs a result. A medical condition ontology with factors and investigations connects to an ontology representing a computational medical guideline. Elemental therapeutic intervention nodes represent drug or nondrug therapies.

### CROSS-REFERENCE TO RELATED APPLICATIONS

This application claims priority of U.S. provisional patent application Nos. 63/773,042 and 63/826,777, filed Mar. 17, 2025 and Jun. 19, 2025, respectively, both entitled “COMPUTATIONAL MEDICAL GUIDELINES FOR DIAGNOSIS AND TREATMENT AND APPLICATIONS THEREOF” (attorney docket Nos. RCVRP007P and RCVRP007P2), which are hereby incorporated by reference. This application incorporates by reference U.S. Pat. No. 11,869,674 (attorney docket No. RCVRP002), entitled “Translation of Medical Evidence into Computational Evidence and Applications Thereof.” This application also incorporates by reference U.S. patent application Nos. 63/789,664, Ser. Nos. 19/189,650, 19/189,664, 19/189,701 (attorney docket Nos. RCVRP009P, P009A, P009B and P009C), entitled “COMPUTATIONAL EVIDENCE WITH THERAPEUTIC INTERVENTIONS AND APPLICATIONS THEREOF,” “COMPUTATIONAL EVIDENCE WITH THERAPEUTIC INTERVENTIONS AND APPLICATIONS THEREOF,” “DETERMINING PATIENT STATE AND NEXT-BEST ACTIONS WITH THERAPEUTIC INTERVENTIONS” and “COMPUTATIONAL EVIDENCE WITH DOCUMENT INDEXING,” respectively.

### FIELD OF THE INVENTION

The present invention relates generally to medical guidelines. More specifically, the present invention relates to medical guidelines that are made computational.

### BACKGROUND OF THE INVENTION

Currently, U.S. healthcare waste is estimated at $1.5 T, and a number of reasons are evident. For one, bias and misdiagnosis are omnipresent, with each of us likely to be misdiagnosed in our lifetime or impacted groups due to bias, and the timeframe for new research to be put into practice is 5-17 years. Further, there is already more knowledge than is humanly possible to stay current on and apply, and only 20% of clinical guidelines are backed by high-quality evidence.

Unfortunately, the burden to be more efficient falls on clinicians, who have reached a ~50% burnout rate. Even though evidence is the fundamental unit of diagnosis, treatment, payer economics, research and regulation, current scientific practice and operational medical knowledge is primarily represented separately as documents and in if-then rules in workflows. This severely limits diffusion, feedback and scalability. And, while powerful, AI architectures based on text alone are not sufficient to represent, process or reason on this knowledge controllably or reliably. And yet further, mapping standard ontologies to documents, for example, as done by existing clinical decision support systems, does not enable controllable use of the information by AI systems; they only provide metadata to the narrative form.

Physician and healthcare needs for evidence and guidance are at an inflection point. Solving this problem is critical to improved (and improving) health outcomes and sustainable healthcare. Current content providers, i.e. publishers, such as DynaMed, BMJ, UpToDate, etc., provide encyclopedia-style medical content look-up for clinicians covering diagnosis, treatment and management of diseases. They assemble the published evidence through expert, human-centric activity—looking at published papers and published society guidelines, sometimes supported by tooling to help human-led assembly activity. These papers are supporting references only. Current best practice takes what are essentially narrative paper documents, even if they are in a digital form, and boils them down to other documents, again in narrative form, that require human beings to interpret their contents. This process is slow, inefficient and generalized, although it is the best available, until now.

Medical evidence is the fundamental form of knowledge underpinning the science and practice of medicine. It is used in the understanding, prevention, treatment and diagnosis of disease. There is no effective way, however, to diffuse, obtain feedback or strengthen evidence across the system. Today, evidence is predominantly narrative and document-based for dissemination. This creates significant problems including: delay—there is a 3- to 19-year lag from bench to bedside; ineffective at scale—manual search is a time-sink in practice; alert fatigue—documents become hard coded rules out of context; and minimal feedback—there is sparse feedback from practice back to research. Further, there is a tendency to put medical evidence into “silos,” that is, a diagnosis or treatment guideline may refer to signs, symptoms and risk factors of a patient in order to direct treatment, but that patient data is stored separately from the guidelines and is not readily available to execute the guideline efficiently. There is no overarching framework for medical reasoning that can make use of all relevant medical evidence in a computationally efficient manner.

Moreover, a medical guideline for diagnosis or treatment can be confusing for physicians because of the wealth of information it contains, especially for a condition difficult to diagnose or difficult to treat. When multiple guidelines exist for a particular condition, it can be more confusing and difficult for a physician to leaf through journals or to access the guidelines online. Further, many guidelines are disconnected in that treatment guidelines for different conditions are kept separate from each other. This does not match reality, where health conditions often affect each other and one guideline should interact and activate another guideline to be considered for the patient. When guidelines do not “talk” to each other, physicians miss important connections, potentially leading to incorrect treatment plans.

Thus, requiring humans, such as physicians, to interpret paper documents or even those documents in digital form is prone to error, physician overload, is time-consuming and does not result in immediate feedback to a patient. Even if medical knowledge and medical guidelines are known to a physician, he or she cannot possibly be aware of all relevant medical knowledge and medical guidelines and cannot process this information fast enough to provide meaningful feedback to a medical query, or to a patient question for diagnosis, treatment, a patient trajectory or other.

There are major challenges with a physician performing the manual process of medical reasoning in his or her mind: humans struggle with complex probability calculations and can make errors with Bayesian reasoning as their cognitive load increases; trying to manage 50 medical information nodes can exceed the typical working memory of a human; the manual process is subjective and will vary between physicians; and clinical settings require fast decisions, making a thorough manual analysis impractical.

Considering the simple example of a single patient with ten observations, 1,000 total nodes in a graph database, we can assume approximately forty nodes after mapping (the ten initial nodes plus thirty nodes inferred). Assuming that the physician needs time not only to make these observations but also to perform the node mapping, create inferences for the other nodes, assign attributes, and perform manual Bayesian calculations, the total time for one patient can be one to two working days for a skilled medical professional, making this manual process for this small number of nodes impractical for routine diagnosis. And the overall error probability can be as high as 50% for significant errors and in complex cases can be much higher. In order to achieve a less than 1% error rate, such a manual process would require a human team of at least six medical professionals per patient case in order to perform: literature review, node mapping, Bayesian calculation, cross-validation, and final consensus. The total time required to reach a result with less than 1% error can be as high as two to three weeks per patient. Assuming that the patient has twenty observations and that there are 10,000 nodes in the knowledge graph, the total time for a patient result balloons to at least two weeks of full-time work for that patient and in order to achieve a less than 1% error is on the order of months. In the context of healthcare, the effort and time involved for even a simple knowledge graph and a patient with relatively few observations is not reasonable and in complex circumstances it is impossible for a human to perform.

Accordingly, a new platform, computer representation and techniques are needed to address the above deficiencies.

### SUMMARY OF THE INVENTION

To achieve the foregoing, and in accordance with the purpose of the present invention, a clinical knowledge graph that connects to computational guidelines is provided.

In general, one embodiment of a computational platform represents a medical guideline in computational form; the platform receives a medical query, processes the medical query using the computational guideline, and then outputs a response in a form that may be useful for any downstream computation. Unlike CQL, the computational platform can be used in any context. Further, a clinical knowledge graph may include one or more of conditions, factors, investigations, therapies and differentials that is connected to the computational guidelines in order to provide a representation for performing end-to-end healthcare, from initial symptoms to treatment.

Computational guidelines provide smart decision support: they can do more than simply provide information. They can allow for software to perform reasoning and predictions on which treatment might work best next or how quickly a condition might progress, using the same medical reasoning a doctor would use. In addition, feedback loops back to guideline publishers. When a guideline is made computational and deployed in a real-world setting, feedback and analytics can be directly measured on the computational guideline; this is not possible with document-based or static online guidelines. And, computational guidelines provide live updates: the guidelines can be updated immediately when new medical evidence emerges. This means doctors always have access to the latest recommendations without waiting for new printed or online materials.

Advantageously, the representation of medical knowledge in a knowledge graph, the representation of medical guidelines as linked nodes in a graph, and the conjunction of the two allow a computer to more efficiently evaluate medical queries and to produce responses that a human cannot perform in his or her mind. By representing medical knowledge in a knowledge graph and medical guidelines that can be traversed as nodes in a graph, the platform described herein can efficiently and accurately process medical information on a computer in order to provide accurate medical results in a realistic healthcare time frame using minimal competing resources.

### BRIEF DESCRIPTION OF THE DRAWINGS

The invention, together with further advantages thereof, may best be understood by reference to the following description taken in conjunction with the accompanying drawings in which:

FIG. 1 illustrates a platform able to diagnose a patient's condition and recommend a treatment using computational guidelines.

FIG. 2 an example of a visualization of a portion of a knowledge graph after any quantity or type of medical evidence has been translated into computational elements.

FIG. 3 provides more detail on the content of condition, factor group, demographics and EDF nodes.

FIG. 4 provides more detail on the content of conditions, investigations group, EDI Result and EDI nodes.

FIGS. 5A and 5B are a visualization of a computational guideline linked to other knowledge graph portions shown herein.

FIGS. 6A and 6B are a visualization of another example of a computational guideline linked to other knowledge graph portions shown herein.

FIG. 7 shows example guideline and determination nodes.

FIG. 8 shows examples of decision nodes.

FIG. 9 shows a drug adjustment node along with various decision result nodes.

FIG. 10 shows examples of drug-related therapy nodes and nondrug therapy nodes.

FIG. 11 shows examples of other specific nondrug therapy nodes and a generic nondrug therapy node.

FIG. 12 shows a portion of a clinical knowledge graph that implements elemental therapeutic interventions along with a portion of a computational guideline.

FIG. 13 is a block diagram showing details on therapy groups, therapy restrictions and elemental therapeutic interventions.

FIG. 14 is another example of a portion of a knowledge graph showing specifics for therapy groups, therapy restrictions and ETIs.

FIGS. 15A-15B are a flow diagram for determining a diagnosis of the patient or a treatment for a particular diagnosis.

FIG. 16 is a flow diagram describing how to compare patient trajectories with a target trajectory.

FIG. 17 is a block diagram showing example use cases.

FIG. 18 is a block diagram showing other example use cases.

FIG. 19 is a flow diagram describing how a knowledge graph may be queried by any of a variety of users and applications, for any of a variety of purposes, and for outputting results in a variety of forms.

FIG. 20 illustrates two different ontologies that are connected, thus creating a computational guideline linked to portions of a knowledge graph.

FIG. 21 illustrates two different ontologies that are connected, thus creating a single, comprehensive graph.

FIG. 22 is a flow diagram describing how patient data inferred from a guideline may be used in a knowledge graph action.

FIG. 23 is a flow diagram describing how to predict a patient trajectory using information obtained from a knowledge graph.

FIGS. 24A and 24B illustrate a computer system suitable for implementing embodiments of the present invention.

### DETAILED DESCRIPTION OF THE INVENTION

It is realized that multiple clinical ontologies and taxonomies exist (e.g. SNOMED-CT), but that these by themselves do not provide the end-to-end computational representation necessary to make evidence and associated medical guidelines operational or computational. A given taxonomy can only be used for limited reasoning: while one taxonomy provides a hierarchy of diagnostic factors and another taxonomy provides a hierarchy of conditions, each provides no information as to how particular factors relate to a condition, how a diagnosis of a patient may be derived using these hierarchies or how treatments may be used to perform reasoning. These are hierarchies of concepts, not relationships, and cannot be used to perform reasoning to link a condition to particular diagnostic factors, to link factors and investigations to treatments, or to link treatments to possible conditions.

We represent medical evidence, the “raw material,” as computational evidence and disclose guidelines that are application ready, computational, diffusible and deployable at unprecedented scale and associated functionality utility across healthcare stakeholders. Computational means that medical guidelines, relating to the understanding of, diagnosis of and treatment of medical conditions, are processable by software applications (including artificial intelligence and other computational models), and combinable with other healthcare data (including patient data and other real-world data), in context and in real time.

Embodiments of the invention are able to combine diagnosis and treatment guidelines and medical evidence into one common ground. The underlying database provides computational elements (e.g., factors, investigations, treatments) found in medical evidence sources in narrative form; the linking of these computational elements with respective conditions and with guidelines allows for computation using the database to provide answers to medical questions. We believe that the only way to scale the delivery of quality healthcare within the world of precision medicine is to make medical evidence computational by converting it into computational elements, by converting medical guidelines into computational guidelines, and then linking the two ontologies. Such computational elements and guidelines can be used in real time and within the technical and cognitive workflow of the clinician and overcome deficiencies in the prior art.

For example, even when a prior art guideline is embodied in a language like CQL (Clinical Quality Language), it is designed to operate in a specific context under a specific set of constraints. That guideline in CQL is not a generalized machine-readable representation of medical evidence that can be used in any context. The CQL guideline is limited in that it has a specific purpose to be used for a specific use case.

### Computational Platform

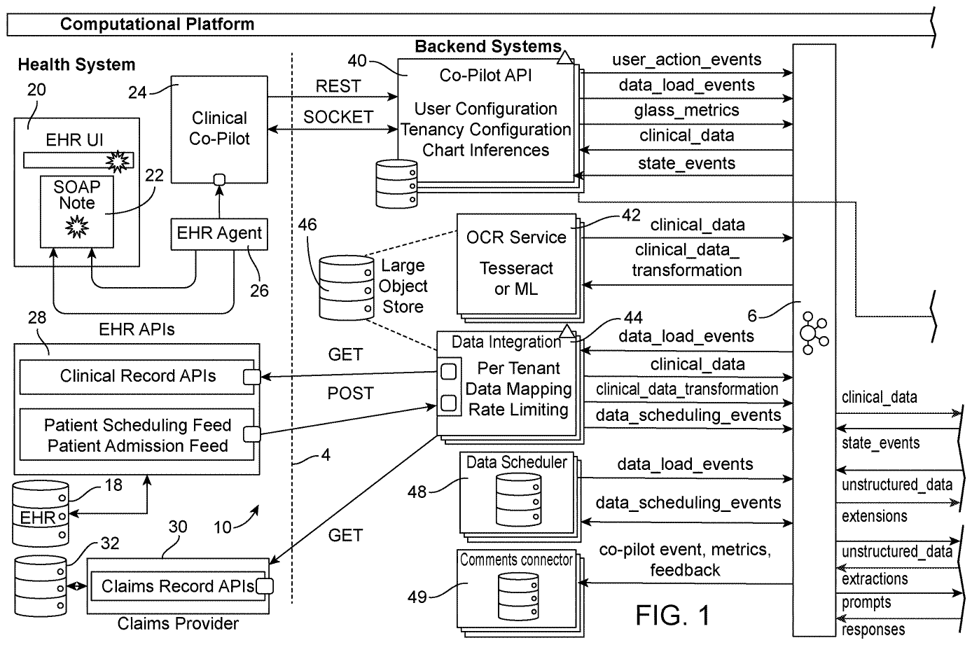

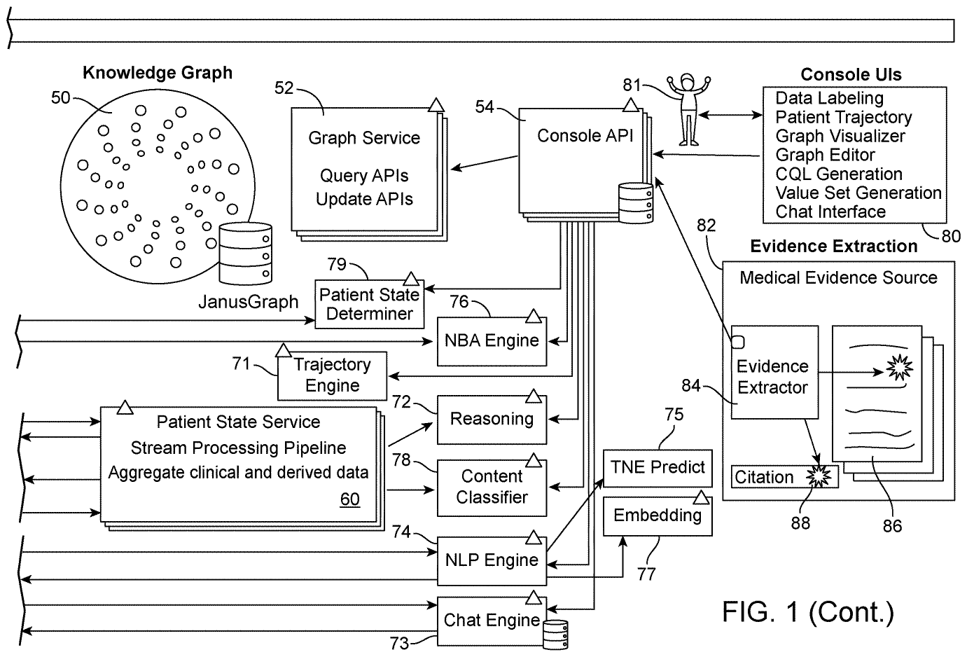

FIG. 1 illustrates a computational platform 10 able to evaluate and diagnose a patient's condition, recommend a next-best action or actions, recommend a treatment or treatments separate from or in addition to a diagnosis, perform diagnosis by treatment, among other tasks, all based upon medical evidence and medical guidelines which have been made computational. Generally, platform 10 may be viewed as including any number of health systems (the user environment) and backend systems, shown separated by vertical dashed line 4. A health system is that hardware and software generally located on customer premises (such as in hospital, clinic, doctor's office, insurance company, or other client of the platform, etc.), while the backend systems are generally that hardware and software located at offices and locations owned by or under order control of the provider of platform 10, such as locations controlled by Evidium, Inc. or located in cloud services such as AWS, Azure, Paperspace, etc. Nevertheless, certain components of the health system (such as an EHR database 18) may be located remotely from the clinician (e.g., in the cloud) and in one embodiment a user interface software application (“Clinical Co-pilot”) 24 is also cloud based and accessed by a clinician using a Web browser on a computer in his or her office. Other components of a health system may also be cloud based. Note that “clinician” used herein refers to medical doctors, physicians, physician assistants, nurse practitioners and other medical professionals qualified to diagnose, treat and care for patients.

An EHR 18 (electronic health record, also referred to as an electronic medical record or EMR) system of record is an external database providing the EHR-stored medical data for the patient, including but not limited to patient demographics, symptoms, investigations, examinations, laboratory test results, signs, diagnoses, treatments or guidelines performed and history. Data may be accessed in both real time and asynchronously pre-encounter or post-encounter. Data is accessed via various methods including but not limited to FHIR (Fast Healthcare Interoperability Resources) standards.

EHR UI 20 is the user interface of the EHR system; the user interface application 24 is a stand-alone application that runs on the clinician's computer (e.g., it is a browser application), although it is capable of being launched from the EHR UI 20 environment with a single sign-on authorization including but not limited to SMART on FHIR and other relevant standards. EHR Agent 26 is an event detector that detects user actions on a computer. It sends messages via user interface 24 to the Co-pilot API 40 which in turn is responsible for retrieving the relevant information from the EHR, automatically detecting any new relevant data, including but not limited to new data added to the EHR system (for example, a new patient record or state change of patient data such as diagnostic data both in real-time and asynchronous modes of operation), and provides that data to application 24, i.e., data that is typically added by a clinician. Shown is a standard SOAP note 22 by which a clinician may enter observations of a patient during or outside of an encounter; other data entry techniques may also be used. EHR APIs 28 is a collection of APIs such as FHIR, USCDI (United States Core Data for Interoperability), CDS Hooks, and vendor-specific APIs which allow EHR agent 26, user interface 20 and other components to communicate with the EHR system. Claims record APIs 30 are any number of APIs used by a claims provider (such as a health insurance company, e.g. UnitedHealth Group, CVS Health, Elevance Health Inc.) to access platform 10 in order to submit queries to knowledge graph 50 and perform other tasks as will be described below, and which may use patient claims information from medical visits and procedures, etc. from a claims database 32.

Clinical Co-pilot 24 is a real-time, fully-automated, possibly hands-free software application and user interface, designed specifically to be the system of interaction by which a clinician may enter patient data, direct that a patient EHR be downloaded to the platform, request a diagnosis or patient state, request a next-best action, treatment, etc. of a patient either during a patient encounter or when the patient is not present, or perform any query of the knowledge graph. For example, application 24 is used by a clinician in real time during a patient encounter (live, by telephone, online via computer, chat via mobile telephone or computer, a video call, etc.) or may be used by a clinician before or after a patient encounter. Application 24 is an interface to any of the backend engines herein which run in parallel with the clinician's actions and do not require any additional input from the clinician. Application 24 may be used as an overlay, as a window tab, or may appear using other user interface techniques.

Application 24 accepts manual clinician entries and selections (e.g., typed input, mouse input, etc.) and may use speech recognition technology (microphone and speech recognition software not shown) as well as live note capture technology to collect real-time patient encounter information. Any verbatim text (either from manual data entry, speech recognition, live notes, from the patient EHR medical chart, etc.) is then converted in real time into factors, investigations, treatments, prior conditions, history, etc., by NLP Engine 74 and matched to nodes in knowledge graph 50 for use by NBA Engine 76, Trajectory Engine 71, Reasoning 72, Content Classifier 78, Determiner 79 and Chat Engine 73 to determine patient state, potential patient conditions, next-best actions, treatments, etc. Application 24 is able to display pertinent positive and negative history from the patient's medical chart in the EHR system that has been extracted by NLP Engine 74 and Reasoning 72.

In one embodiment, application 24 is an application integrated into the EHR environment via relevant standards such as a SMART (Substitutable Medical Applications, Reusable Technologies) on FHIR application for EHRs (such as Epic and Cerner), and is integrated into the clinical workflow. In another embodiment, application 24 is a standalone system, integrated with relevant external data sources. The suggestions from application 24 are provided for expert consideration and not as a replacement for the clinician. Thus, application 24 provides real-time (or outside of a patient encounter), in-context differential diagnosis, next-best-action and treatment suggestions to validate and expand clinical consideration, along with query capability.

OCR Service 42 provides mechanisms to convert images of text to digital text strings for further analysis. Data Integration Service 44 mediates access to external EHR services including the translation from the abstract data model used by the CEP to the specific data models of the EHRs. Data Scheduler Service 48 provides a mechanism that enables patient charts to be analyzed and cached prior to a patient's visit thus facilitating better real-time performance. Comments Connector 49 is a software module that collects user feedback for different systems that can be present in the form of reviews, upvote and downvote ratings, and other similar mechanisms. Communications channel 6 is a software layer that communicates information between application API 40 (and Comments Connector 49 and components 42, 44, 48, 49) with components 60, 74, 76 and 73.

Knowledge graph 50 is a database or data store that stores the extracted information from the medical evidence sources after that information has been translated into computational elements. Knowledge graph 50 may also be referred to as a clinical knowledge graph, a database, a knowledge base, an evidence base, an inferential database, or an inferential database of computational elements. Any of a wide variety of types of databases or data stores may be used to store this information such as a relational database, an object store, an object-oriented database or non-relational database (NoSQL). In one embodiment, knowledge graph 50 is implemented using a JanusGraph graph database having nodes and arcs (or links), the nodes containing medical evidence of different types (e.g., conditions, differential diagnoses, diagnostic factors, risk factors, investigations, treatments, drug and non-drug therapies) and the arcs (or links) indicating linkages, causality, flow, or other (made clear by the context) between the nodes. Depending upon the context, these arcs or links may be unidirectional or bi-directional. In one particular embodiment, the knowledge graph is undirected and thus allows traversal in any direction depending upon the use case. For instance, while predicting conditions given a set of investigation results and diagnostic factors, we can traverse up from EDIs and EDFs to condition nodes, whereas if a user wishes to find all diagnostic factors of a particular condition we can traverse down from condition nodes to EDFs. Further, the knowledge graph may contain nodes and edges, and the edges themselves may also hold any of the medical evidence listed above.

Evidence extraction 82 is performed to extract textual medical evidence from any of a variety of sources for representation within the knowledge graph 50. Any of a wide variety of medical evidence sources 86 may be used and include: National Library of Medicine; JAMA Rational Clinical Evidence; BMJ Best Practice (or other British Medical Journal databases); SNOMED-CT (Systemized Nomenclature of Medicine-Clinical Terms); LOINC (Logical Observation Identifiers, Names, and Codes) database; CPT (Current Procedural Terminology) codes; StatPearls content; and other online sources such as Merck Manuals, WebMD, NORD (raredisease.org), the CDC (for endemic information), the WHO, RxNorm (for medications), First Data Bank (FDB, for drug-drug interactions), NICE and other accredited associations that publish treatment guidelines such as American College of Cardiology etc.; as well as laboratory test reference ranges, drug interactions (drug-drug, drug-condition, drug-food), clinical dictionaries, and content on rare diseases. The evidence extractor 84 can extract clinical items from any textual data source; it maps extracted concepts to these code systems or ontologies.

Typically, the sources used are in electronic form, such as a JSON file, XML file, or other electronic format representing textual information. If they are Web sites, then they may be marked up in HTML. The textual information may be free-form text, may be in narrative form, may be semi-structured text (e.g., text within flow diagrams, decision trees, etc.). In addition, evidence-based medicine sources are used and include a collection of external data, such as datasets of clinical support reference content for healthcare professionals which contain best practices, evidence-based research relevant for clinical decision support including diagnosis, treatment and prognosis.

An evidence extractor 84 is a system which extracts relevant text from an evidence source and delivers it to NLP engine 74 for recognition of the clinical concepts within the text. Evidence extractor 84 identifies and retrieves appropriate contextual clinical data from evidence sources discussed above and delivers this data to any component requiring clinical concept validation. By surfacing authoritative medical evidence that supports system decisions, it helps build user confidence in the system's recommendations while also providing explanation directly from the evidence source.

The extractor facilitates the selection, extraction, and delivery of relevant text to the NLP engine 74 for the recognition of domain-specific concepts. By way of example, when text related to a particular medical condition is input the extractor extracts the condition, its diagnostic factors, risk factors, investigations, any differential diagnoses, rankings, treatments, guidelines, etc. and delivers those to the NLP engine. The extractor 84 also extracts a citation or citations 88 associated with the extracted text. The extractor may be implemented in forms such as a stand-alone application or a Web browser plug-in function. After the NLP engine extracts structured concepts from unstructured or narrative text, the evidence extractor fetches the appropriate condition, its diagnostic factors, risk factors, investigations, any differential diagnoses, rankings, treatments, guidelines, etc. associated with the structured concepts.

The extractor can also allow users to author custom evidence of their own. An example use case is a medical association that publishes treatment guidelines; the association can use the Graph Editor tool to build a new computational treatment or diagnosis guideline and annotate it with their custom evidence. Use of an NLP engine for evidence extraction is optional, for example when the extracted text is already in structured form or when a human-in-the-loop reviews and edits the extracted text in order to place it into structured form.

Console user interface (or interfaces) 80 is used by a person 81 and includes a Graph Visualizer for viewing knowledge graph 50, a Graph Editor by which the person views or reviews information extracted from the medical evidence, corrects errors, adds factors (or other nodes), perform groupings, etc., thus changing and improving the extracted evidence before it is stored into knowledge graph 50. The Graph Editor may also be used to create, add to, or edit a computational guideline and store it within the knowledge graph 50. A CQL Generation interface allows the user to generate executable CQL (or other) code by providing natural language descriptions of any custom query the user wishes to execute on a patient database. A Value Set Generation interface allows a user to generate value sets of any desired elements of the knowledge graph. A Chat Interface allows the user to query the knowledge graph in natural language (e.g., via an LLM) and retrieve context-relevant clinical information along with evidence. A Patient Trajectory interface allows the user to find potential future trajectories of a patient along with analyses of risk scores and evidence that explain the predicted trajectories.

NLP (natural language processing) engine 74 may include any number of NLP processes (such as an NLP pipeline), each may use a model or not, and each may choose to store information in a wall cache. The NLP processes that may be used include but are not limited to: Word Segmenter, Sentence Segmenter, Negation Cues, Negation Scopes, NER (Named Entity Recognition), Relationship Prediction, NEL (Named Entity Linking), Phrase Extractor, Composite Matcher, Embedding Matcher, Graph Matcher, NLI and Long-to-Long Matcher. The NLP engine may also include models or functions for extracting temporality cues and scopes, experiencer cues and scopes, sex cues and scopes, and age cues and scopes.

NLP Engine 74 takes in some textual narrative from a medical document, extracts medical concepts and then translates them into appropriate nodes in the clinical knowledge graph (EDFs, EDIs, ETIs). It also plays a key role in gathering patient state from unstructured textual data (like patient notes, discharge summary, etc.). Thus, NLP Engine 74 is used not only for extracting information from medical evidence sources, but also for interpreting a patient's chart and outputting diagnostic factors, risk factors, results of investigations, treatments and other items of a patient's medical history that may be matched to nodes within the knowledge graph or used to implement a computational diagnosis, treatment guideline, or other query within the course of traversing the comprehensive graph. Preferably, the NLP engine is trained in the domain of medical concepts.

NBA Engine 76, Reasoning Service 72, Content Classifier 78, Chat Engine 73, Determiner 79 and Trajectory Engine 71 are all software modules used by platform 10 to perform various tasks. NBA Engine 76 is used with any use case that requires cross-condition prediction given an existing patient state. Its primary purpose is to determine high risk or likely conditions and then suggest the next-best investigation or observation to conduct given the patient's current state. It may use structured data sources (like EHR FHIR data, claims data, etc.) and/or the NLP Engine to extract patient's clinical concepts from unstructured data.

Patient State Service 60 functions as an interface layer that ingests patient data from diverse sources and in diverse format (e.g., FHIR records from an EHR, patient data input from application 24, claims information from insurance providers). It transforms this heterogeneous data into system-specific representations tailored to the internal requirements of the software modules used (e.g. 71-73, 76, 78, 79). Patient State Service 60 also acts as a caching layer that serves to enhance real-time operation for already-analyzed patients and to control access to the various functions provided by the platform. By way of example, during a diagnosis, determination of the next-best action, recommendation of a treatment or guideline, or other query, etc., patient state service 60 keeps track of the patient's state (including the factors, investigations, treatments, etc. that the patient has presented with over time or that are presented in the encounter). Patient state service 60 may also send the patient state (including any inferred factors, investigations, treatments from the Reasoning Service) to the Content Classifier 78 (or other diagnostic classifier) which performs a straight classification using condition scoring.

During determination of the patient state, service 60 takes the known factors, treatments and investigations and sends those to Reasoning Service 72 in order to infer other factors, treatments and investigations using an ontology link in the corresponding EDF, ETI or EDI. Using the appropriate ontology, service 72 and walks the relevant ancestor-descendant chains. Reasoning Service 72 also understands the ancestor-descendant relationships of clinical tests, e.g. CBC with differential is a parent of CBC (complete blood count) is a parent of Hematocrit and White Blood Count.

For example, given that the patient has “acute fever” we may also infer that the patient has the broader factors “fever” and “change in body temperature.” Not all conditions are described in the literature with the same level of specificity, and similarly, patients may be very specific in their description of symptoms, e.g. a patient suffering from botulism might report very specific left-upper quadrant abdominal pain, whereas the evidence for botulism does not localize the abdominal pain to a specific area and its diagnostic factor is “abdominal pain” only. Without the inference from LUQ abdominal pain to UQ abdominal pain to “abdominal pain” (found by traversing the relevant ancestor-descendant chains of an ontology), a diagnostic factor for botulism would be missed.

Chat Engine 73 performs dynamic conversation orchestration against an input medical query from a clinician with optional additional context as can be fetched from service 60 or supplemental database integrations. The primary use of the Chat Engine is to allow humans to interact with patient records (coming from either EHR, notes or by copying and pasting information in the chat, etc.) contextualized with healthcare knowledge coming from the clinical knowledge graph. One embodiment of the Chat Engine uses an LLM and a graph query processor. Chat Engine 73 executes appropriate reasoning sequences, including calling NBA Engine, Reasoning, Determiner and NLP Engine where required to return an answer grounded in the contents of the knowledge graph.

TNE Predict module 75 performs temporality, negation and experiencer attribute tagging on elements of the knowledge graph. Embedding module 77 transforms medical concepts (factors, diagnoses, investigations, procedures, treatments, etc.) into vector representations. These embeddings capture semantic relationships between medical concepts, enabling efficient similarity-based retrieval. Trajectory Engine 71 is used by an insurance company (or other) to traverse a patient trajectory in a guideline using patient claims data. Modules 40, 44, 52, 54, 60, 71-74 and 76-79 (flagged with a small triangle) generate responses based upon clinical data structured in the knowledge graph, ensuring semantic consistency, factual accuracy, and contextual relevance across all system operations.

### Computational Data

In general, the platform makes medical evidence computational including medical conditions, factors, investigations (including laboratory tests and examinations) and therapeutic interventions (i.e., drug and nondrug treatments). A medical condition may be any condition, a disease, an orthopedic anomaly, an adverse drug reaction, or a drug-to-drug interaction.

A diagnostic factor is a factor that the physician can observe, detect or measure quickly or have the patient describe to him or her during an office visit (traditional signs and symptoms), while an investigation is a laboratory test or an examination performed by a specialist; both are more sophisticated and take more time. By way of example, diagnostic factors include: “patient has a headache,” “patient skin appears yellow,” “patient reflexes appear normal,” etc. Thus, the value of a particular diagnostic factor may be a simple yes or no, a numerical value, a numerical range, the presence or absence of the diagnostic factor, an observation (e.g., “appears normal”), or other results such as a specific physiological description like “the nasopharynx is symmetric.” The platform also distinguishes between diagnostic factors and risk factors; as is known in the art, a diagnostic factor is caused by a condition, whereas a risk factor may contribute to, or partially cause, a condition. Risk factors may include gender, age, family history, smoking, medications, etc., and examples include: “sex is male,” “patient smokes,” and “family has a history of colon cancer.”

Typically, investigations require either the collection and processing of some sort of biological sample (a laboratory test) or a physiological analysis using some sort of device (an examination). Laboratory tests may include fluid analysis (e.g., blood panel, urine, semen, and CSF), and examinations may include diagnostic imaging (CT, MRI, X-ray, PET, ultrasound, endoscopy) and pathology analysis among others. By way of example, the result of a laboratory test is “CBC blood test shows reduced hemoglobin level” and the result of an examination is “MRI of the pelvis appears normal.” Thus, the result of a particular laboratory test or examination may be a simple yes or no, a numerical value, a numerical range, the presence or absence of a particular item, an observation (e.g., “appears normal”), or other result. Depending upon the implementation, vital signs, e.g. heart rate, blood pressure, oxygen saturation, etc. may also be considered as investigations-they can be performed quickly, by clinicians with a lower level of education or training, and for little to no cost. Generally, although not always, investigations will be the ones with numbers or numeric ranges as results whereas diagnostic factors indicate the presence or absence of something. Some observable factors may be considered investigations.

Our design uses EDFs (elemental diagnostic factors) to represent factors, which are computational elements resulting from translating human-readable evidence. Clinical factors described by evidence-based medicine and medical guidelines are translated into EDFs. Specifically, an EDF is a clinical factor that may be evident in a number of conditions, either as caused by a condition (e.g., signs, symptoms, vital signs) or as a risk factor that may contribute to the cause of a condition. Traditional evidence-based medicine considers factors on a condition-by-condition basis; EDFs enable these factors to be disambiguated across conditions by the platform. In cases where the same factor applies to multiple conditions (but may be referred to by different terminology), EDFs enable those like factors to be understood as having the same meaning. Thus, EDFs provide a common representation of a potential disease state of a patient, useful in diagnosis as well as in next actions and treatment. As an example, EDFs disambiguate clinical factors where one human-readable text describes “neck stiffness” for one condition and “nuchal rigidity” for another; the EDF “stiff neck” replaces both, thus using a single EDF across both conditions. Another factor is possibly described in different sources as “being feverish” or “high temperature,” all meaning the same; thus the single EDF “fever” is used.

EDFs may also be used to describe higher-level factors than the atomic elements, for example the factor group “GI symptoms” is defined to include the EDFs “nausea,” “abdominal pain” and “vomiting.” Another example is the factor group “constitutional symptoms,” defined using the EDFs “fever,” “malaise,” and “weakness.” Another example is a medical reference that lists the diagnostic factor “pelvic pain” under the condition “ectopic pregnancy.” The factor “pelvic pain” is quite broad and we extract from the narrative in the reference the very specific EDFs of “acute pelvic pain,” “right-lower quadrant pain,” “lower abdominal pain,” and “pelvic pain” which then allows “pelvic pain” to be a factor group and used for other conditions (e.g., BPH, benign prostatic hyperplasia which is a male condition). The following EDFs related to pelvic pain may be used: acute pp, burning pp, chronic female pp, chronic pp without obvious pathology, constant pp, cyclic pp, dull pp, female pp, hx (history) of chronic female pp, hx of cyclic pp, intermittent pp, no female pp, no pp, pp on penetration, sharp pp, throbbing pp, which are all distinct concepts used in various conditions, each being disambiguated across conditions in the knowledge graph.

EDFs are part of a computational evidence ontology in a clinical knowledge graph, which includes other relevant concepts related to conditions, investigations and therapeutic interventions. EDFs are defined in a way such that each can be applicable across any condition and may be used in any type of consultation such as diagnosis, determination of a next-best action or actions, treatment, prediction, etc. Each EDF may also be connected to an ontology or taxonomy, including but not limited to SNOMED to allow inferences to be drawn (using Reasoning Service 72) and to provide standardized synonyms. A particular EDF may be found in a one-to-one relationship with another clinical factor in an ontology or taxonomy, although many EDFs are created anew and do not exist in other ontologies or taxonomies.

Each EDF may also be associated with a variety of properties specific to a particular condition such as an age and sex restriction (e.g., the factor is only applicable to persons of a particular age and only to male or female patients). These condition-specific properties may be held in a demographics node as shown below (or on an edge between the condition and the EDF) but separate from the EDF. Other conditions having that EDF may have different demographics nodes that are specific to that condition, and demographic nodes need not be present for a condition. Thus, EDFs remain elemental, independent, and can be used computationally across conditions. EDFs may also have properties defined such as temporality of the factor (historic or present), experiencer of the factor (patient or family member) and negation (whether the clinical factor does not occur or is not present). Synonyms may also be defined in an EDF and allow the EDF to be recognized across different sources that use different terms.

EDFs are made independent by placing various properties that are specific to different conditions into different data structures linked to both a particular EDF and to the particular condition. In this manner, an EDF remains independent and may be used computationally across different conditions, yet within the context of a particular condition these properties are taken into consideration. In one particular embodiment, properties of an EDF relevant to a specific condition are held in a factor group linked to that specific condition and to the EDF (or held on an edge between the condition and EDF).

Clinical factors of the patient state (from the EHR or other input source) are translated and matched to the relevant EDF in the course of diagnosis, next-best-action, treatment, or other query. This enables the clinical state of the patient to be determined, for next-best-actions to be determined, and for diagnosis and other computations to be performed across all conditions, all using the same knowledge graph.

In a similar manner, the platform uses elemental diagnostic investigations (EDIs) to represent independent laboratory tests and examinations which may be used for computation across conditions in a knowledge graph. Similar to EDFs, certain properties of a laboratory test (e.g., a result, units, range, etc.) specific to a particular condition are not held within the EDI itself (thus ensuring its independence) but are held separately (in a results node, or on an edge) yet still linked to a specific condition. Further, an investigation group specific to a particular condition represents any number of laboratory tests or examinations and has its own properties defined as well. As with EDFs, condition-specific properties are not present within the EDI, but are held in a results node, in an investigation group node, or on any edge or edges between a condition and the EDI.

And further, the platform uses elemental therapeutic interventions (ETIs) to represent independent pharmacological (drug) and nondrug therapies (or treatments) also used for computation across conditions in a knowledge graph. Certain properties of a therapeutic intervention specific to a particular condition are not held within the ETI itself (thus ensuring its independence) but are held separately (in related nodes, or on an edge) yet still linked to a specific condition. A therapy group specific to a particular condition represents any number of therapies and has its own properties defined as well. Thus, condition-specific properties are not present within the ETI, but are held in related nodes or on any edge or edges between a condition and the ETI.

Each EDF, EDI or ETI includes a link to its corresponding entry in a suitable ontology for uniquely identifying the node, performing inferences, finding synonyms, matching, etc. Embodiments may use a clinical ontology with pre-coordinated and post-coordinated expressions from SNOMED for EDFs, use logical observation identifiers names and codes (LOINC) for EDIs, use RxNorm or FDB for ETIs (pharmaceuticals), use ICD or CPT, or use UMLS or OMOP for reasoning (these last two contain crosswalks from one ontology to another). Certain nondrug therapy ETIs can also link to SNOMED, e.g., ETIs for genetic and cellular therapy can link to body structure and substance hierarchies in SNOMED. Other ETIs link to other suitable ontologies.

Each of these EDFs, EDIs and ETIs have characteristics that differentiate them from the prior art. Each is atomic: EDFs, ETIs and EDIs can represent the most specific fundamental elements of healthcare knowledge. Each is independent but can be placed in context: EDFs, ETIs and EDIs are independent with respect to any condition, but related contextual nodes (e.g., groups, demographics, results, restrictions) describe the dependence on, or relationship with, linked medical conditions. Each is composable into a representation in a knowledge graph: any healthcare artifact (notes, research papers, documents, queries, etc.) can be linked to multiple EDFs, ETIs and EDIs and hence can be represented as a composition of these computational elements in a knowledge graph. Each is made computational because each may be used in: deterministic reasoning (using logic built into these constructs and into the knowledge graph); traversal operations of a knowledge graph with defined edges and nodes (e.g., a query expansion by finding all contextually-related search terms); and probabilistic reasoning (group nodes include how an element is probabilistically related to a condition, hence allowing for probabilistic calculations and inferences). Because of these characteristics, each may be used horizontally across many use cases: patient state determination, next-best action recommendation, non-contextual or contextual querying of the knowledge graph, and document indexing and representation within the knowledge graph.

Translation of Medical Evidence into Computational Elements

Medical evidence such as medical conditions, the diagnostic factors that indicate those conditions, the risk factors that may cause conditions, and the investigations (laboratory tests and examinations) that indicate conditions, may be extracted from a variety of sources and translated into computational evidence as described in U.S. Pat. No. 11,869,674 (attorney docket No. RCVRP002). Similarly, diagnosis, treatments for conditions, along with the related decisions, results from those decisions, therapy choices, and therapy ingredients as herein described, may also be extracted from a variety of sources (using the techniques described in U.S. Pat. No. 11,869,674 or by using other techniques) and translated into computational guidelines such as the respective treatment nodes, ETIs, decision nodes, decision result nodes, drug adjustment nodes therapy choices nodes, and therapy ingredient option nodes that are described below. By way of example, the Graph Editor is a tool that allows users to build the different components of a guideline, i.e., to construct the various types of nodes and edges as is described herein.

Example Knowledge Graph with Conditions, Diagnostic Factors, Investigations

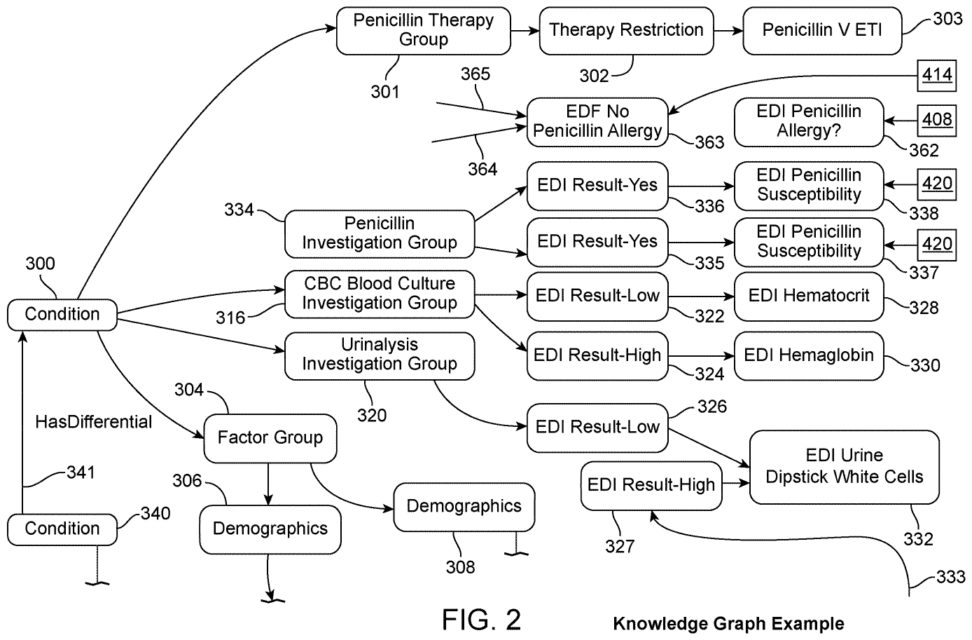

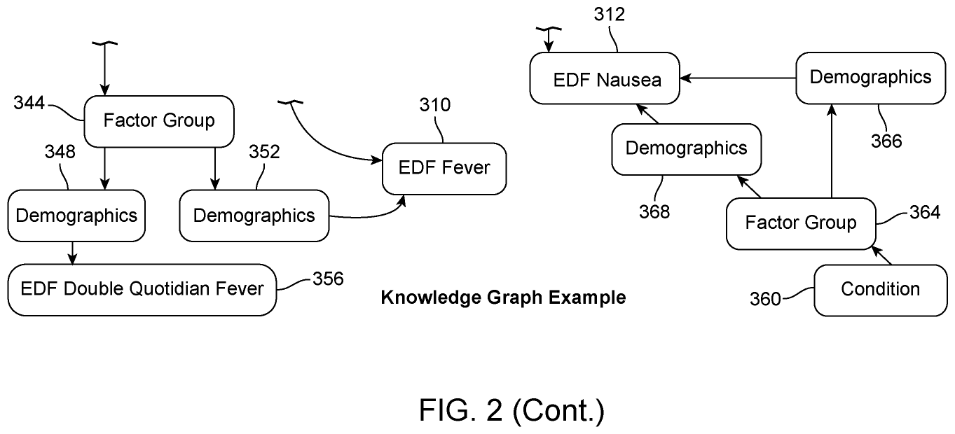

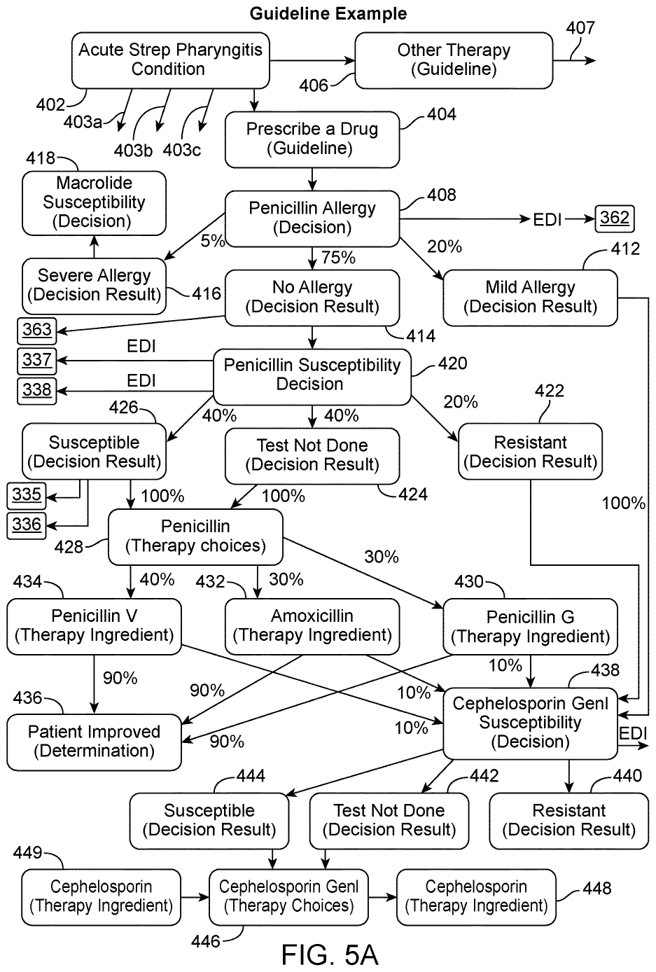

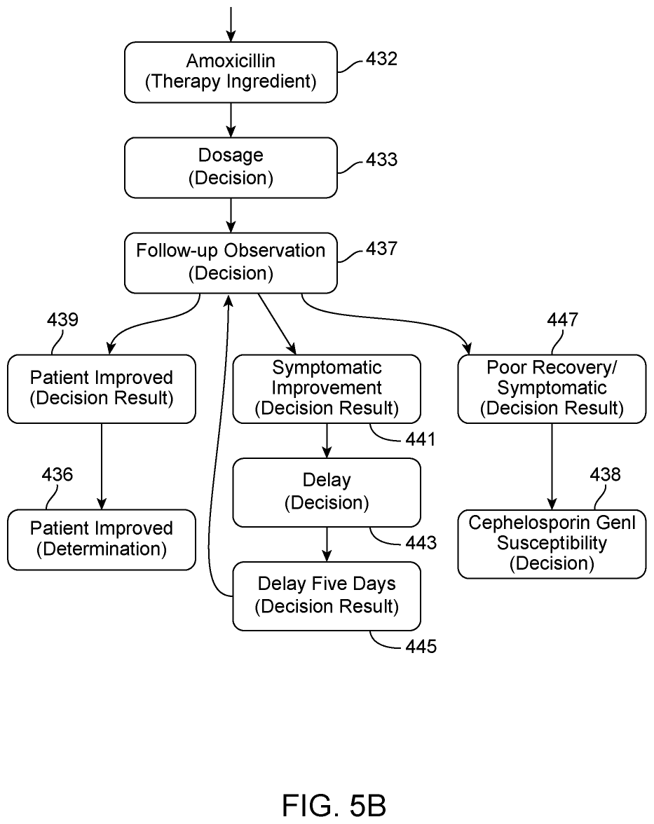

FIG. 2 is an example of a visualization of a portion of a clinical knowledge graph after any quantity or type of medical evidence has been translated into computational elements. The portion shown in FIG. 2 is linked with the guidelines shown in FIGS. 5A, 5B, 6A, 6B and 12 as is described below to form a comprehensive graph having any number and type of nodes and links. There may be any number of such nodes of medical evidence in a clinical knowledge graph, hundreds, thousands, hundreds of thousands, millions or even billions of such nodes. There also may be at least any number of such nodes, e.g., at least hundreds, at least thousands, at least hundreds of thousands, at least millions or even at least billions. There also may be any number of nodes in computational guidelines, typically a computational guideline will include anywhere from dozens of nodes up to around a few thousands of nodes, up to approximately 10,000 nodes, although some guidelines may have up to 100,000 nodes or more. Typically, there may be up to approximately 1,000 computational guidelines in a comprehensive graph, although many thousands of guidelines are possible. There also may be at least any number of such guideline nodes, e.g., at least hundreds, at least thousands, at least ten 10,000, or at least hundreds of thousands. There also may be at least any number of clinical and guideline nodes in a comprehensive graph, e.g., at least hundreds, at least thousands, at least hundreds of thousands, at least millions or even at least billions.

Shown are specific conditions 300, 340 and 360, each having any number of factor groups or investigation groups. In this example conditions 300, 340 and 360 are specific medical conditions that are not named for purposes of the example. Condition 300 has a factor group 304 including two EDFs, fever 310 and nausea 312, each of those associated with a particular demographics node 306 or 308. The demographics nodes are inserted automatically by the system and there may be a single demographics node for each EDF node. Linkages between condition 300, factor group 304, demographics and EDF nodes are provided based upon information gleaned during translation of medical evidence. In this simple example, the input text revealed that the factors fever and nausea are diagnostic factors for condition 300. Similarly, linkages between condition 300 and its investigation groups, result and EDI nodes, as well as linkages between condition 300 and its therapy group nodes 301, therapy restriction nodes 302 and ETI nodes 303 are also based upon the translation of medical evidence.

Condition 340 has a factor group 344 including two EDFs, double quotidian fever 356 and fever 310, each of those associated with a particular demographics node 348 or 352. In this simple example, input text revealed that the factors double quotidian fever and just plain fever are diagnostic factors for condition 340. Note also that the EDF fever 310 is a diagnostic factor for two conditions, as is EDF nausea 312. Condition 360 has a factor group 364 including one EDF nausea 312 having a demographics node 366 and a demographics node 368. For example, there may be two demographics nodes for nausea node 312 because for this particular condition the factor is only present for the very young (node 368) or the very old (node 366). Other conditions may require two or more demographics nodes for a single EDF (or multiple Result nodes for an EDI). In another example, for the condition, “abnormal uterine bleeding,” there will be two demographics nodes for the factor “women at extremes of reproductive age.” One demographic node will have “females 12-19” and the other will have “females 40-60.” Advantageously, each EDF has one or more demographics nodes linked to it that hold condition-dependent properties that further define how and when a particular factor is present for a specific condition. For instance, if the EDF fever 310 only occurs because of condition 300 in children under 12 years of age, that information is present within demographics 306.

Condition 340 also has a related differential diagnosis indicated via link 341 of condition 300. This means that if condition 340 is determined to be present in the patient (as described below) that a differential diagnosis is also condition 300. But, there is no corresponding differential diagnosis link from condition 300 to condition 340, thus indicating that, for this example, condition 340 is not a differential diagnosis of condition 300. Link 341 is added based upon extracting factors and investigations for a particular condition, e.g., the text may say “condition A is often mistaken for condition B.” For example, influenza may be confused with RSV because they have the same signs and symptoms. Person 81 using the console will add link 341 if condition 340 might also be condition 300 but will only add a similar link from 300 to 340 if condition 340 might also be mistaken for condition 300. Such links may also be added by the platform automatically during translation by the natural language processing.

Condition 300 also has two investigation groups, CBC blood culture 316 and urinalysis 320, both also produced by portions of an evidence source being input via evidence extraction. Similar to factors, the single laboratory test EDI urine dipstick for white cells 332, is at the end of the linkage, with its possible result, EDI Result low 326, being between nodes 320 and 332. The EDI Result type of node is similar to a demographics nodes in that an EDI Result node provides a result specific to its indicated condition and is indicative of that condition only when the result has a particular value, such as “low” in node 326.

The other investigation group of condition 300 is CBC blood culture 316 and is an example of an investigation group which may include many specific tests, each having a particular value which is indicative of the condition, if that value is present. Although a CBC blood culture may include many results, only shown are the specific tests EDI hematocrit 328 and EDI hemoglobin 330, each having a particular result 322 and 324. Again, the specific laboratory tests 328 and 330 are only indicative of condition 300 if the results 322 and 324 are as shown.

Nodes 322-326 hold the specific results of a test that indicates when condition 300 is present and EDI nodes 328-332 contain a description of the actual test itself. As with EDFs, an EDI may be linked to many different conditions, each such link including an EDI result node between the EDI and the condition, in this fashion, an EDI is independent of a condition, yet it may have a result which indicates a particular condition. By way of example, node 326 links to condition 300, and a different result node 327 of EDI node 332 with a result of “high” links 333 to a different condition (not shown). Thus, each EDI node may link to any number of EDI result nodes. Or, the result of “low” in node 326 may link to two or more different conditions, each link indicating that node 326 indicates the condition to which it is linked.

Condition 300 also links to any number of therapy group nodes, in this example to penicillin node 301 representing the penicillin class of drugs that may be used to treat this condition 300. Each therapy group node in turn links to any number of therapy restriction nodes, such as node 302, representing restrictions and contraindications for any number of ETI nodes, such as node 303, representing a specific prescribable drug (or nondrug therapy) that may be prescribed to treat condition 300. In general, restrictions inform when a particular therapy of an ETI should not be prescribed, recommended or given, or to which demographics should not be given the therapy. ETI nodes are also atomic, independent, do not include condition-dependent properties, and may be linked to any number of conditions as well as to nodes of a medical guideline as shown and described below.

Condition-dependent properties for EDFs, EDIs and ETIs are held respectively in factor group and demographics nodes, in investigation group and result nodes, and in therapy group and therapy restriction nodes. For example, each factor group node such as 304 holds a ranking indicating the likelihood that fever 310 is caused by condition 300. Separating out specific demographics and the ranking from an EDF node allows each EDF node to be truly elemental, independent and atomic in the sense that each EDF node can be referenced and used by any condition; the specifics as to how a factor manifests because of a particular condition and its ranking are held in the factor group and demographics nodes (or on edges between the condition and the EDF node), not in the EDF node. By way of example, if fever 310 presents itself in a different way for different ages depending upon whether the patient has condition 300 or condition 340, these details are held in demographics nodes 306 and 352 rather than within EDF node 310. And, because fever 310 may be ranked differently for condition 300 and condition 340, these rankings are held in nodes 304 and 344 as well. Thus, a fever 310 in the context of condition 300 is a definition of fever 310 within that node plus its properties within nodes 306 and node 304.

For certain conditions with bimodal distributions there will be two demographics nodes between fever, for example, and condition 300 (e.g., when the fever only occurs in very young children and in very old people). In another example, a patient with condition 340 may exhibit double quotidian fever 356 as well as plain fever 310, each being a separate EDF node with its own particular demographics and ranking. This separation of different fevers into different EDF nodes, each having different demographics and ranking allows more accurate diagnosis and reasoning. Because a person with condition 340 may exhibit double quotidian fever and a patient with condition 300 will not, it is advantageous to keep these two elemental factors separate yet still allow the factor “fever” to be shared by both conditions are shown.

In a similar manner, an investigations group holds a ranking for its linked EDIs, and each EDI result node holds results of the investigation specific to the condition to which it is linked. Similarly, a therapy group holds an indication ranking and the line of treatment for its linked ETIs, and a therapy restriction node includes a condition interaction for the condition to which it is linked.

### Computational Elements Detail

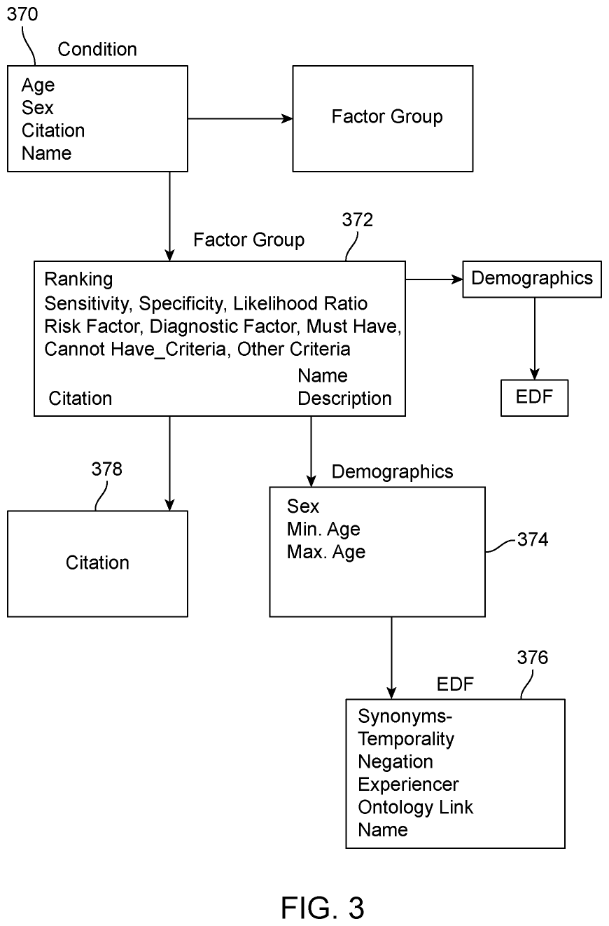

FIG. 3 provides more detail on the content of condition, factor group, demographics and EDF nodes. As shown, condition node 370 may include properties for that particular condition such as a name, age (e.g., the condition can only occur in individuals having an age between 12 and 55), sex (e.g., the condition may only pertain to males or females), and a link to a citation describing that condition may also be present. Factor group 372 may include a ranking (applicable to each of the EDFs to which it is linked), a weight value (which may be used instead of the ranking) which may incorporate sensitivity, specificity and the likelihood ratio, a type which indicates whether the EDFs linked below the factor group are diagnostic factors or risk factors, and “must have” and “cannot have” criteria indicating whether or not the EDFs of this factor group must be present for the condition to occur or must not be present. Other criteria include a certain number of the linked EDFs must be present for the condition to occur (e.g., “at least two of the below diagnostic factors must be present”), or a points system may be used, each of the below factors receiving a number of points and a minimum must be reached before the condition is known to occur. Also included is a link to a citation 378 providing support in a medical evidence source for the use of the factors of the factor group in indicating the condition. Each factor group may also have a name (e.g., gastrointestinal symptoms) and a brief description taken from the evidence source.

The ranking of the factor group indicates how likely it is that the particular factors of the factor group have been caused by its linked condition (in the case of diagnostic factors), or how likely is it that the factors have caused the medical condition (in the case of risk factors). A ranking may be expressed in an evidence source using numerical values, or words and phrases that refer to the likelihood that a group of factors is caused by particular condition or have caused the condition and may be directly extracted from the evidence source or may be added by person 81 reviewing that source.

Other condition dependent properties include sensitivity, specificity, likelihood ratio, incidence, and prevalence among other elements. These all may be combined with the ranking to provide a weight value for the factor group, the weighting being a function calculated outside of a factor group, although the weight value may be stored in the factor group if desired. A weight value may be calculated in a wide variety of manners, including using the ranking only; another technique is to use specificity and sensitivity to calculate the likelihood ratio (the probabilities of people with the factor having or not having the condition).

The condition-dependent properties within a factor group node may be added in a variety of manners. Some of the attributes may be automatically added when the factor groups are added to the knowledge graph based upon output from the NLP engine when it recognizes the input text. Or properties may be automatically determined by using external ontologies or taxonomies. Or person 81 may add properties to a particular factor group when reviewing the clinical concepts during extraction.

Demographics node 374 includes the demographics of EDF node 376 which must be true for that factor to indicate condition 370. By way of example, if the factor of EDF node 376 is only present in certain genders, or only below or above certain ages in the context of condition 370 (i.e., the factor can only indicate the condition if the patient meets the demographics), then that information is held in node 374. Holding these demographics related to a particular condition also ensures that the EDF remains independent and applicable to any of a number of conditions.

EDF node 376 may include only the basic information needed to represent a particular factor (e.g., fever, headache, nausea) such that node 376 is elemental and independent and may be used as a factor in other conditions. By only holding the basic information in node 376, and by holding properties for that factor that are only relevant to condition 370 in nodes 372 and 374 (or on edges between nodes 370 and 376), node 376 can be used as a factor for many conditions.

For all of the elemental nodes of a knowledge graph described herein (EDFs, EDIs, ETIs), such a node may be implemented using basic information such as a unique link (e.g., a code, unique identifier, URL, URI, etc.) to an entry in a suitable ontology which defines the concept (e.g., factor, laboratory test, examination, or therapeutic intervention) represented by the node. This entry is a unique, standardized code which acts as a single source of truth in a knowledge graph. Even though particular ontologies and taxonomies are used, any of the ontology links herein may link to a suitable ontology, taxonomy or hierarchy depending upon the concept. In addition to or instead of an ontology link, the node's unique name or other unique identifier (e.g., a UUID or universally unique identifier) may be used to uniquely define the concept.

Not only does an ontological link provide a unique definition of the concept, it also provides extra information, logic, and data about the concept that may not be present in the graph itself. For EDFs, the link allows us to reason and perform inferences in the SNOMED hierarchy. Similarly, for EDIs we can infer a result of one test as a result of another more generic test; for example, by knowing that White Blood Cell count of an individual is abnormal, we can infer that their CBC panel is abnormal as well—the ontology allows us to traverse and connect WBC count with CBC panel. For drug-based ETIs the ontology allows us to find “label” information for a drug which helps us compute a dosage for a patient, predict future adverse reactions, and obtain other information from the drug label. For example, using the ontology, an adverse reaction to penicillin helps us predict with some likelihood that an adverse reaction to carbapenems also exists given they both are β-lactams. A link to at least one ontology or taxonomy using a unique code references that same factor (or test or drug) within the known ontology providing the ability to traverse the hierarchy of that ontology either up or down to perform inferential reasoning from the node using the medical ontology or taxonomy.

A node may also include its name (e.g., fever), synonyms (e.g., elevated temperature, pyrexia, febrile, feverish, etc., derived from the evidence source or from input by person 81), a description, temporality (T, a binary value indicating whether the factor was experienced in the past or now), negation (N, a binary value indicating whether the factor is present or not present), and experiencer (E, a binary value indicating whether the factor is experienced by the patient or family member). These values for TNE may be stored within node 376 or in a separate node linked to node 376, or not present. Further, given that there are three binary values, there may be as many as eight EDF nodes 376, each having a different value using the combination of these three values. Each of these eight EDF nodes is also independent and each may be used by many different conditions. For instance, an EDF node for fever may list temporality is now, fever is present, and experiencer is the patient. A second EDF node fever may list temporality is now, fever is not present, and experiencer is the patient. The other six nodes vary the other two attributes T or E.

An EDF, EDI or ETI is also made computational by virtue of its context in the knowledge graph. Its links to other nodes and conditions provide the ability to traverse between elements and conditions and allows reasoning capabilities. Further, logic for use in this reasoning is included within related nodes. For EDFs this logic is included within factor groups (e.g., a ranking) and demographic nodes (e.g., to whom the linked condition may apply); for EDIs the logic is within the investigation groups (e.g., a ranking) and result nodes (e.g., a result which indicates a condition); for ETIs the logic is within therapy groups (e.g., an indication ranking) and restriction nodes (e.g., when not used therapy). As mentioned before, this logic need not necessarily be held within particular nodes but may be held upon edges or in an associated location between the elemental concept and the condition.

Even though a factor group and a demographics node are shown between an EDF node and its indicated condition, it is optional to include these two nodes. If a particular diagnostic or risk factor is not dependent upon a patient's gender or age in order to indicate its linked condition, then a demographics node is not relevant and need not be present. Further, any properties of a factor group node such as ranking, risk factor or diagnostic factor, must-have, cannot have, etc. can be held on the edge that links a condition to its EDF, in which case an explicit factor group node is not needed. And diagnostic factors and risk factors may be held in different types of nodes, and the “must have” and “cannot have” criteria may also be represented by different types of EDFs. For a condition that does not have groups of factors that can be grouped conveniently, it may be simpler to link a condition directly to that factor without having any intermediate factor group node.

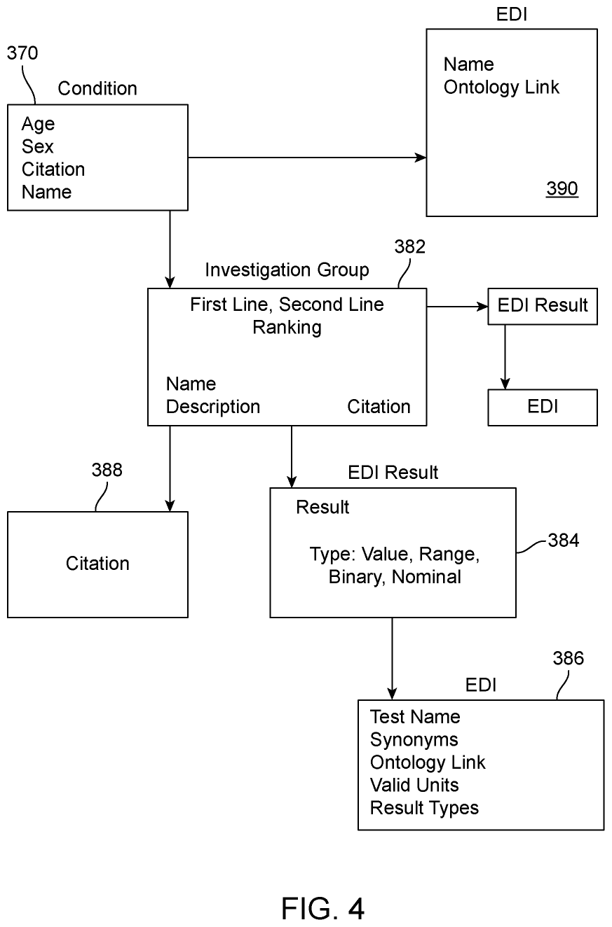

FIG. 4 provides more detail on the content of investigations groups, EDI Result and EDI nodes. Investigations group 382 includes a name and description as above and a ranking (applicable to each of the EDIs to which it is linked) representing the likelihood that the linked condition is indicated when the result in the EDI result node is present. Also included is whether the group is first line, second line or emerging, etc., thus ordering the various investigation groups, which ordering may be adjusted by person 81. While an EDI node represents a laboratory test or examination that may be shared between many different conditions, its corresponding EDI Result node holds the particular result of that test which indicates that its linked condition is present. An EDI node may very well have two or more EDI Result nodes, each having a different result which links to a different condition.

Each EDI Result node 384 includes the actual result (e.g., “low,” 32, greater than 100, in-between 50 and 100, yellowish, normal, etc.) from the laboratory test or examination that indicates the condition 370, units of the result, possibly reference range values, and the type of the result: a value, range, a binary, an ordinal, nominal, etc. Each EDI node 386 may include the test or examination name, synonyms, a link to one more taxonomies or ontologies (such as LOINC) using a unique code for that particular test or examination, and possible result types and valid units for that test or examination. Note that the types listed for a particular EDI Result 384 are likely a smaller subset than the result types listed in node 386. In another example, an EDI 390 may link directly to a condition and the condition-dependent properties and results may be stored in an associated location between condition 370 and EDI 390 such as on an edge. Thus, all condition-dependent properties indicating what is a valid result to indicate a linked condition are held outside of an EDI. Thus, each individual EDI node (whether for a laboratory test or examination) remains elemental and independent and may be used computationally to indicate various conditions. An EDI node may include only the basic information such as an ontology link, and optionally the other information.

Because the results of some laboratory tests and examinations can vary for the same test or examination (e.g., some machines have different reference range values, some are machine-readable and some are hand-readable, hospitals may use different tests for the same concept, different machines refer to the same test by a different name), it is advantageous to keep particular properties and their values within an EDI Result node rather than within the EDI node itself, thus ensuring that an EDI node remains independent and may be used for computation for any condition. Other investigations such as examinations are handled in the same manner.

Even though an investigation group node and a result node are shown between an EDI and its indicated condition, it is optional to include these two nodes. Properties of an investigation group may be held on an edge or edges between a condition and an EDI. For a condition that does not have groups of investigations that can be grouped conveniently, it may be simpler to link a condition directly to that investigation without having any intermediate investigation group node. Even the actual result in a result node may be held on an edge, such as an edge between an investigation group and EDI, or between a condition and an EDI in the case where investigation group and result nodes are not present.

Similar to the relationship between EDFs nodes and demographics nodes, while an EDI node such as 328 holds a laboratory test that may be shared between many different conditions, its corresponding EDI Result node 322 holds the particular result of that test which indicates that the particular condition 300 is present. EDI node 328 may very well have a different parent EDI Result node having a different result which links to a different condition or to no condition at all.

Returning now to a discussion of the investigation groups of condition 300, note that each has one or more EDI Result nodes below which hold results of laboratory tests or examinations that are specific to a particular condition. Similarly, penicillin investigation group 334 links to two different penicillin susceptibility tests 337 and 338, each having an EDI result node 335 or 336 indicating whether a culture tested from a patient is susceptible to penicillin or is resistant; in this case, each result node 335, 336 stores the property of “susceptible.” Not shown are the other EDI result nodes for each EDI 337 and 338 which store the property of “resistant” or “test not done.” In this example, penicillin investigation group 334 does not link to a condition; in the context of using tests 337, 338 to determine if a particular drug should be used, the tests need not necessarily link to a condition. Investigations may be used for tasks other than condition diagnosis such as treatment guidance, therapeutic response monitoring, etc. Hence not all investigation groups need to be linked to a condition; in fact, an investigation group is not strictly necessary, and investigations 337, 338 and their EDI results need not link to anything else, other than decision nodes.

For any given laboratory test or examination, there may be many laboratory tests that may be used in order to make a similar determination: each test may be a slight variation in how a result is determined, some tests may be machine-readable, others may be hand-readable; one clinic or hospital may use a test different from another; the particular machine being used for the test may refer to the test by a different name, even though the result provided is the same; or different tests providing the same result may be referred to by different names for any variety of reasons. In another example, an EDI 390 may link directly to a condition (or need not link to any condition or result) such as when the EDI represents whether the patient has a drug allergy (see discussion of node 408 below).

Above is described how an EDF or EDI may include an ontology link and below is described how an ETI and various nodes in a computational guideline may each also include an ontology link. Such an ontology link for each of these nodes is a unique code, unique link, or similar unique reference that uniquely identifies the medical concept represented by the node in the relevant ontology. By way of example, an EDF representing the diagnostic factor “double quotidian fever” includes an ontology link to the ontology SNOMED-CT that identifies that particular fever within the ontology. Or, an ETI representing a prescribable drug includes an ontology link to the ontology Rx-Norm that identifies that drug. Or, a nondrug therapy node representing a particular procedural therapy (for example) includes an ontology link to the relevant ontology that uniquely identifies that particular procedure.

### Example of Connected Ontologies

Above and below are shown portions of a clinical knowledge graph having a variety of nodes such as conditions, EDFs (along with factor groups and demographics), EDIs (along with investigation groups and results), ETIs, and the various links or edges between these nodes. Any number of other ontologies representing computational guidelines (such as for diagnosis or treatment) and its nodes may also be linked to the above-described knowledge graph creating connected ontologies and a larger comprehensive graph. Any ontology for a computational guideline can thus leverage the EDFs, ETIs and EDIs of the above clinical knowledge graph, thus creating a comprehensive graph that enables end-to-end diagnosis and treatment of a patient. Within such a comprehensive graph, the conditions, EDFs, EDIs, therapy nodes and ETIs may be referred to as the clinical knowledge graph (i.e., a knowledge graph of clinical knowledge), while an ontology of nodes representing a computational guideline that links to the clinical knowledge graph may be referred to as the computational guideline. Any number of computational guidelines may be linked to the clinical knowledge graph as shown and the computational guidelines are preferably implemented as is the clinical knowledge graph, i.e., in any suitable database or data store and preferably using the Janus Graph graph database. Examples of a comprehensive graph representing both a clinical knowledge graph and computational diagnosis and treatment guidelines, are presented below. There may be any number of nodes in a comprehensive graph, including hundreds, thousands, hundreds of thousands, millions, billions or more.

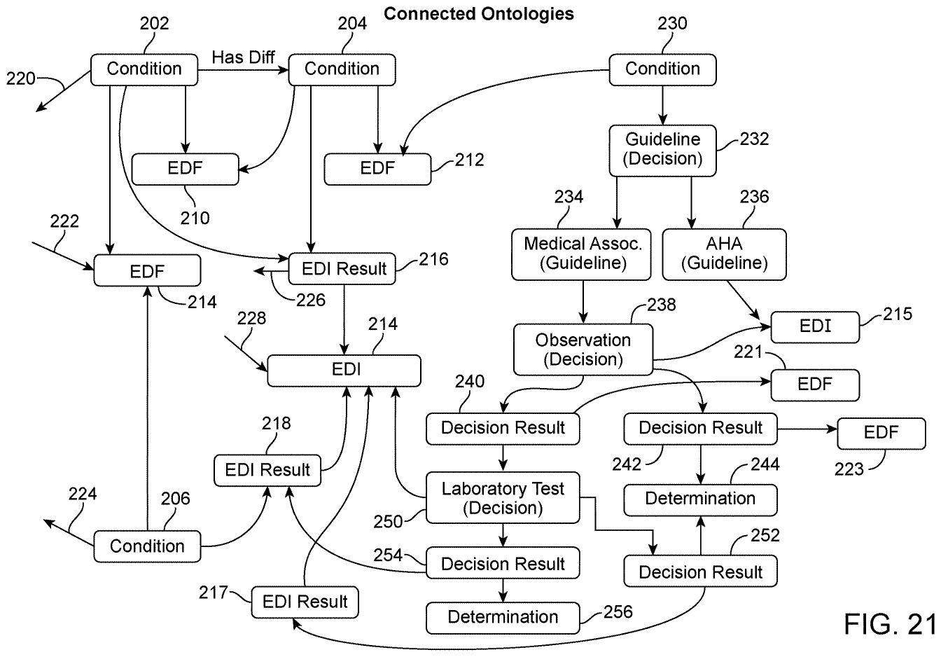

FIG. 21 illustrates two different ontologies that are connected, thus creating a single, comprehensive graph. Connecting any number of computational guidelines to the clinical knowledge graph provides information (via EDFs, EDIs, ETIs and their related nodes) to a guideline which is not present in the guideline itself. While a clinical knowledge graph contains medical evidence as herein described, a computational guideline describes steps to diagnose or treat. While a clinical knowledge graph may be traversed beginning at any node and in any direction, a computational guideline may only be meaningfully traversed downward, i.e., from a condition to an eventual determination in order to diagnose or treat. For purposes of identifying nodes, though, a guideline may be traversed in other directions over links between nodes. A clinical knowledge graph includes nodes representing medical evidence or medical concepts such as conditions, factors, investigations, results, therapeutic interventions, etc. that generally describe how these concepts are related; it does not include patient-specific information such as may be found in an EHR, clinician notes, insurance claims database, or other source of the patient's medical history. A computational guideline, while it includes nodes providing reasoning for making a diagnosis or treatment, does not include nodes found in the clinical knowledge graph which hold medical evidence.

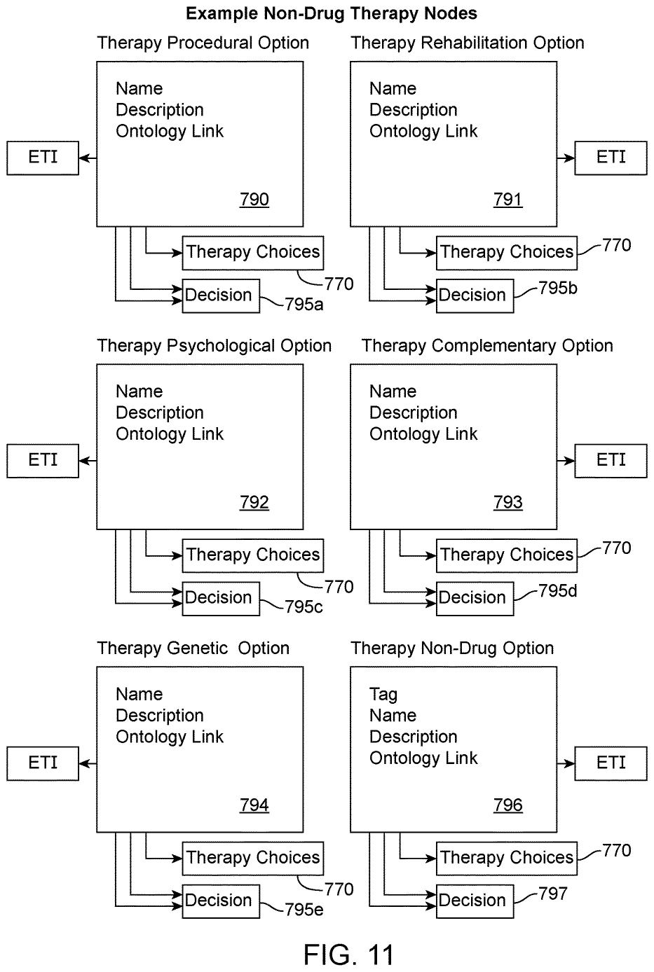

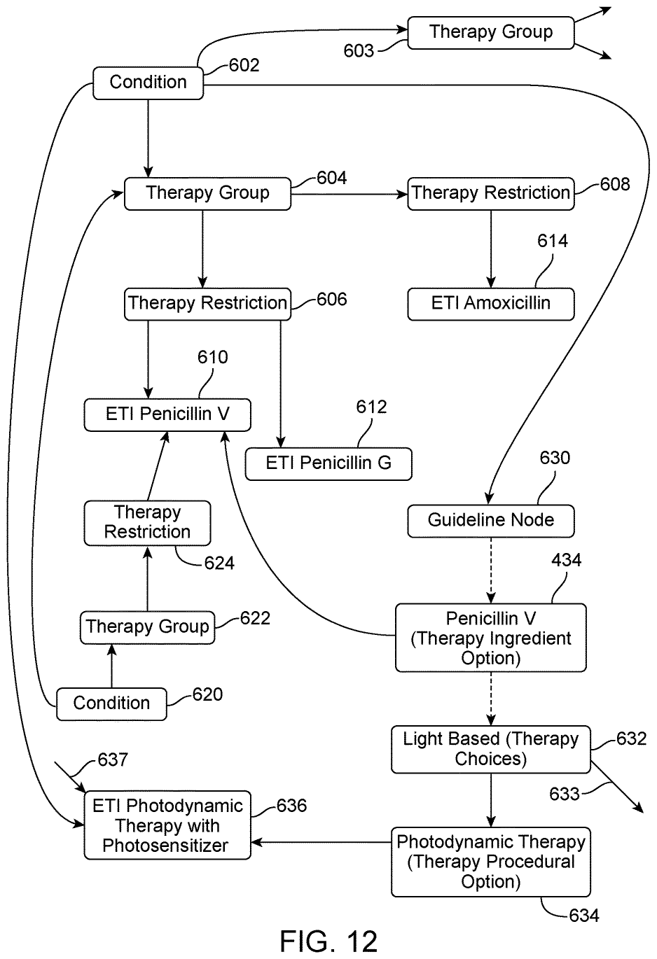

Shown generally on the left-hand side of the figure are nodes 202-223 representing a clinical knowledge graph described in the previous figures for performing diagnosis and determining next-best actions, among other tasks. This portion includes three condition nodes 202-206, four EDF nodes 210-214, 221, 223 representing different diagnostic or risk factors, EDI nodes 214, 217 representing particular investigations that inform a condition, two result nodes 216, 218 representing different results from that investigation, and an EDI node 215 not directly linked to any condition. As shown, any condition may link to any number of factor or investigation nodes, a factor node may indicate any number of conditions, and an EDI node may indicate any number of conditions via its result nodes). As shown at links 220, 222, 224, 226, 228, any of the conditions, factors, investigations or results shown may link to other portions of the clinical knowledge graph not shown in this drawing (e.g., links to ETIs). As mentioned above, it is not strictly necessary that explicit factor group and demographics nodes are located between an EDF and a particular condition, or that explicit investigation group and result nodes are located between an EDI and a particular condition. FIG. 12 shows how nodes in a computational guideline may link to ETIs in a clinical knowledge graph.

The right-hand side of the figure generally are nodes 230-256 representing an example of a computational guideline for diagnosis, these nodes and computational guidelines being described in greater detail below. Briefly, at node 232 a decision is made to choose a particular medical association guideline 234 and an observation of the patient is needed at 238. Advantageously, node 238 links to EDI node 215 which is used to obtain the particular observation of a patient needed to make decision 238.

If the result of that decision leads to node 242 then a determination may be made in 244 that the condition is present in the patient. If the result is as in node 240, that leads to decision node 250 which requires a particular laboratory test. Advantageously, node 250 links to investigation node 214 representing a laboratory test or examination which is used to obtain (or order) the result from that laboratory test from the patient's EHR. If that result leads to node 252 (using information from node 217) then the determination in node 244 is confirmed, while if the result leads to node 254 (using node 218) then the determination in node 256 may conclude that that condition is absent in the patient.

Thus, linking of nodes in a computational guideline for diagnosis or treatment into a clinical knowledge graph in which diagnostic factors, risk factors, investigations and interventions are represented as EDFs, EDIs and ETIs leads to many advantages. Preferably, observation, allergy, and laboratory decision nodes link to EDIs, observation and allergy decision result nodes link to EDFs, and laboratory decision result nodes link to EDI result nodes. As shown, condition 230 is any condition within the clinical knowledge graph and it may link to any number of EDFs, EDIs, or ETIs, as well as other differential diagnosis conditions, etc. Condition 230 links to the guideline below it and may also link to any number of other conditions, factors, investigations, etc. as described herein. Further, even though FIG. 21 shows a computational guideline for diagnosis generally on its right-hand side, any computational guideline such as for treatment, or for diagnosis and treatment (including those described below) may be linked to EDFs, EDIs and ETIs in a clinical knowledge graph as explained herein.

Example Computational Guideline with Decision and Related Nodes

FIGS. 5A and 5B are a visualization of a computational guideline linked to a clinical knowledge graph portion shown herein, thus forming a comprehensive graph. In this example, a particular condition node 402 is linked to particular guideline nodes 404, 406 to facilitate diagnosing or treating a patient having (or suspected of having) that particular condition. As will be explained in greater detail below, a guideline node may be the entrance to a portion of the computational guideline which describes a flow for performing diagnosis of the patient, for performing treatment of the patient, or for performing a mix of diagnosis and treatment within the same flow. In this simple example, we assume that the condition 402 is known and thus choose a guideline 404 which describes a treatment. Thus, FIG. 5A illustrates a treatment guideline made computational; in one embodiment, it is NBA Engine 76 which executes the computational guideline.

As shown, condition 402 may link in 403a to any number of other condition nodes (via a “has differential” link), may link in 403b to any number of other EDF nodes (either directly or indirectly via factor group and demographics nodes), may link in 403c to any number of other EDI nodes (either directly or indirectly via investigation group and result nodes), or may link to ETIs, thus linking FIG. 5A with any other knowledge graph portions. Linked below condition node 402 are any number of guideline nodes 404, 406 each describing a particular guideline for treating a condition; there may be a decision node in between a condition and the various guidelines in order to select one of the guidelines (described below), or, as in FIG. 5A, there may be a predetermined order in which the guidelines are applied. As will be explained in greater detail below, a particular guideline node is the entrance into a computational guideline for describing how a patient will be diagnosed or treated using the guideline.

In another example, assuming that the condition is hypertension, a decision node below the condition node may allow different guidelines to be selected, e.g., the first line may be exercise and diet modification while a second line may be any number of different types of diuretics, meaning that two guideline nodes are below a decision node. Or, assuming a different condition in which various drugs may be used, a guideline node for this condition indicates that a drug should be prescribed, and there may be any of a number of different drugs that a clinician might pick as the first line. Accordingly, there may also be a decision node directly below a “Prescribe a Drug” guideline node that decides which drug to try first before deciding whether an allergy is present or not, i.e., before moving an allergy decision node.

In this example, a treatment guideline 404 for prescribing a drug is the preferred, or first-line, treatment, and it will be used first. Another possible therapy (or second-line treatment) begins at guideline node 406. In this example, penicillin is the preferred drug to treat this condition, so control moves first to decision node 408. In order to determine if the patient has an allergy to penicillin or not, shown is a link from node 408 to an EDI 362 in the clinical knowledge graph of FIG. 2, for example, an EDI representing the investigation “Does the patient have a penicillin allergy?” thus prompting the guideline to perform this investigation (if not already done) and attempt to match patient data to the results. In this example, EDI 362 does not link to any result nodes (EDIs need not link to result nodes), but may link to any condition such as anaphylaxis. Observation decision nodes also link to EDIs.

Each allergy decision result node 412-416 links to a corresponding EDF in the clinical knowledge graph: a “Mild Penicillin Allergy” EDF, a “No Penicillin Allergy” EDF 363, and a “Severe Penicillin Allergy” EDF (only EDF 363 is shown along with link 414). EDF 363 links via 364, 365 to any number of condition nodes, either directly or via demographic or factor group nodes; the other two EDFs may also link to condition nodes. Thus, although EDI 362 EDI does not have a direct link to EDF 363, it is indirectly connected via the decision result 414 of its corresponding observation decision 408.

The guideline then matches the relevant patient data (either using the patient's EHR data or by prompting the clinician to ask the patient about their allergy status) in order to match the patient's allergy status to one of nodes 412-416. Depending upon the patient state, patient availability, the use case, etc., the system may reference the EHR, may prompt the clinician to query the patient or to perform the investigation of the EDI. There may be a mixture of referencing the EHR, querying the patient or performing the investigation in any given guideline.

Depending upon whether this patient has a penicillin allergy or not, control then moves to one of the decision result nodes 412-416. If there is a mild allergy, then control moves to decision node 438 where a different drug may be tried. If there is a severe allergy, then control moves directly to decision node 418 which is a different class of drug, and that node performs a susceptibility laboratory test similar to that performed in node 420, and the process continues from there with respect to that particular drug. If there is no allergy, then control moves to laboratory decision node 420 which references or requests a laboratory test (in this case, a culture test) to determine whether this particular culture from the patient's throat is susceptible to the penicillin family of drugs. Links from node 420 lead to the EDIs 337 and 338 of FIG. 2 which are different laboratory tests for testing if the particular strain of bacteria in the patient's culture is susceptible to penicillin, and if so, this means that penicillin will be effective in treating the patient. As shown, node 420 links to EDIs 337, 338 and node 426 links to EDI results 335, 336.

Matching patient data to nodes in the computational guideline may be performed in different manners. By way of example, from node 420, the backend engine executing the guideline takes the patient's EHR data with the result of a specific test and attempts to match it to one of the EDIs 337, 338 and their corresponding results 335, 336 (e.g., using ontology links and codes specific to the particular test). Hence, if patient data suggests that penicillin susceptibility test 337 was performed (rather than test 338) and the result was indeed “susceptible”, the backend engine maps that result to nodes 337 and 335, and then to the “susceptible” result node 426. Since it is possible that a single EDI and EDI result pair may map to multiple conditions, by connecting the patient state to the EDI and EDI results in the knowledge graph we can infer more information than is available in the patient state. Preferably, patient data is matched to EDI and EDI result nodes as a pair, rather than mapping first to an EDI and then to a particular EDI result, although that technique may also be used. In another example of matching, patient data may first be mapped to an EDI and from there to the appropriate EDI result. Or, the EDI may be matched to patient data, and that result in the patient data for the particular EDI may then be matched to the appropriate decision result node of the computational guideline. Other techniques for matching may also be used.

Depending upon whether the patient is present in person, whether this flow is being executed upon an existing EHR, or whether or not any of these laboratory tests have already been performed, the results from laboratory tests may be obtained in different manners. In one example, a culture has already been obtained from the patient, the test has been performed and the result is present in the EHR from where it is obtained. Or, the patient walks into the clinic, a rapid strep test is performed, a culture is taken, and the result is entered into the patient's EHR at that time, which may be available hours or days later. Or, the clinician orders such a test as he or she is examining the patient. Or, the patient or clinician knows or suspects that the drug will be effective (or has a strong desire to use it or try it), and enters the result of “susceptible” into a user interface in order to arrive at node 426. In any case, if the result of either test 337 or 338 indicates “susceptible” or if the test is not done, then control moves to node 428. If the results of any test indicate that the culture is resistant to penicillin, then control moves to node 422 and then on to node 438 in order to try a different drug.

Therapy Choices node 428 indicates any number of nodes which represent the penicillin group of drugs. Below node 428 are any number of therapy ingredient option nodes 430-434 indicating particular options in the penicillin group of drugs that may be used by the clinician. At this point, these drug options are output to the clinician in some form and the clinician chooses one of the drugs to prescribe and then monitors the patient's progress. These options may be output to a clinician via Chat Engine 73 and ultimately clinical copilot 24. At some point in the future, if the patient improves then a determination is made at node 436 that the patient has improved and no other drug need be prescribed. To input the patient's improvement the clinician may (a) update the patient state in the EHR and the integrated APIs 28 will update the patient state in patient state service 60, or may (b) provide an update on the patient's improvement via clinical copilot 24.

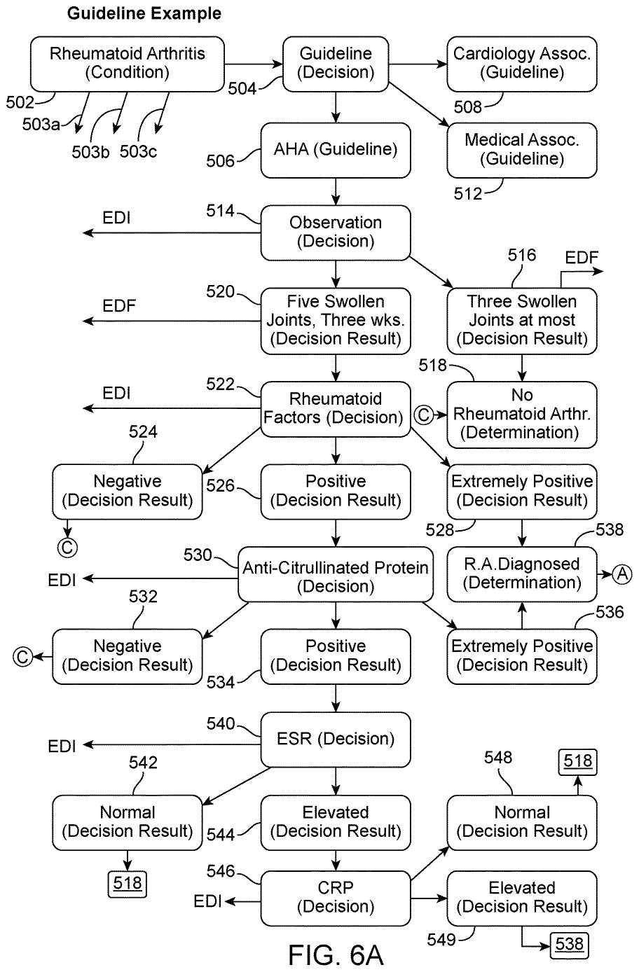

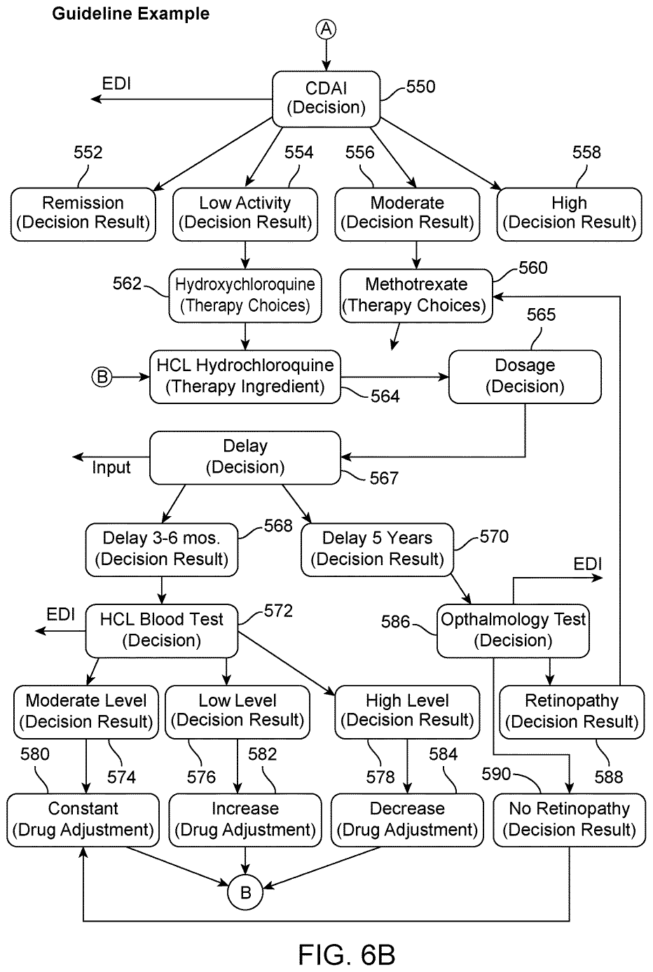

FIG. 5B shows a particular embodiment for handling dosage of a drug, follow-up care, and patient improvement. As shown, in this case of strep pharyngitis, each drug (e.g. 432, 434, etc.) is preferably followed by a dosage decision (433), and each dosage decision is followed by a follow-up observation decision node (437). The dosage regimen is calculated and stored using node 433 and recommended to the clinician as described in FIG. 6B. The drug is prescribed and the patient is monitored and observations of the patient may be input by the clinician as described above.

A patient can demonstrate one of the three decision results to this observation decision node: patient improved 439, symptomatic improvement 441 (i.e., patient condition improved slightly but he or she is not fully well yet, symptomatic remission) and poor recovery, patient still symptomatic 447. In case of patient improved 439 the treatment should come to an end and hence this result is connected to the determination node 436 as shown. In case of the second result 441, the clinician will typically give the patient more time to recover and ask him or her to come back for a follow-up after some delay (and hence the cyclic edge in the diagram after five days). In case of the third result 447, the clinician will suggest the next line of treatment (Cephalosporin in this case, node 438) given that the first line has failed to address the illness. Depending upon the condition, it is possible to have different decision results 439, 441, 447 and those decision results may lead to other types of nodes such as drug dosage adjustment nodes, etc.

In addition to prescribing one of the three drugs 430-434, the clinician may also ask the patient (or consult the EHR) to determine if any of these drugs are contraindicated (i.e., they would have an adverse reaction with another medication the patient is taking), and if so, then control moves to node 438 which is a decision node similar to node 420, in which it is determined whether the culture from the patient's throat is susceptible or resistant to the drug cephalosporin Gen1, which is the first choice in the cephalosporin group of drugs. (Control may also arrive here from node 412 if the patient has a mild allergy to penicillin.) Similar to the flow discussed above at node 420, any number of laboratory tests may be used to determine if the patient culture is susceptible or resistant to this drug; flow moves to any of nodes 440-444 depending upon the results, and if the culture is susceptible or the test not done, then control moves to the cephalosporin Gen1 therapy choices node 446. Any of the therapy ingredient options may then be prescribed using nodes 448-449 and control may continue as discussed above with reference to nodes 430-434. If contraindicated, then control may move to another node, cephalosporin Gen2 susceptibility, another decision node, in which it is determined whether the culture is susceptible or resistant to this particular drug, and the flow may continue, if needed, on down to cephalosporin Gen3, etc. In general, if the patient has an allergy to a particular drug or that drug is contraindicated, the clinician may choose a drug affecting broader types of bacteria.

In general, if it is determined that a culture is resistant to a particular drug, the patient does not improve using a particular drug, or that a drug should not be prescribed for whatever reason, then control may move to different drug within guideline node 404, or to a different therapy not involving drugs under a different guideline node (e.g. node 406). For example, control may move to a different class of drugs at node 418 if the patient is allergic to penicillin or if the patient is allergic to the cephalosporin class of drugs. Or, depending upon the guideline, at node 447 if a patient is not improving then control may move to a node that prescribes a nondrug therapy.

At any point during execution of a computational guideline, the clinician may desire to try a different therapeutic intervention, to request a factor or investigation not in the flow, or to choose a different guideline, he or she can do so in the following manners: manually-he or she uses clinical co-pilot 24 to navigate to another guideline or to jump to a different entry point within the same; automated-if the clinician EHR is integrated into the computational platform via APIs, he or she can simply continue with the alternate investigation or entry point, the patient state will update automatically and the backend engine will automatedly detect the new entry point in the same or alternative guideline.

Example Computational Guideline with Decision and Related Nodes

FIGS. 6A and 6B are a visualization of another computational guideline linked to a clinical knowledge graph forming a comprehensive graph. In this example, a condition 502 “rheumatoid arthritis” is suspected. FIG. 6A includes nodes representing a guideline for diagnosing the condition (at nodes 538, 518), while FIG. 6B includes nodes for treating that condition. Thus, these figures illustrate how a guideline that includes diagnosis and treatment may be made computational.

Accordingly, a computational guideline may represent a guideline for diagnosing a condition, treating a condition, diagnosing and treating a condition, diagnosing a condition by treating it, or any combination of these, as well as other guidelines such as administrative guidelines (e.g. situation-based decision making for administrative staff), protocol guidelines (e.g. a guideline to be followed during a clinical trial), preventive guidelines, and cohort-based guidelines. Not every guideline will include diagnostic components and similarly, not every guideline will include treatment components, although some guidelines may include both diagnostic and treatment components.

Typically, information about the patient's state is already known (such as patient and family history, investigations already performed and known diagnostic and risk factors, treatments performed), and this patient state may be determined by a clinician using manual techniques, by using an automated technique such as that described in U.S. Pat. No. 11,869,674 (attorney docket RCVRP002), or by using any other suitable technique. Assuming that rheumatoid arthritis is suspected, this guideline is initiated and will lead to an update of the patient state, i.e. confirming the diagnosis or ruling it out. If confirmed, then flow may continue into another portion of the guideline to implement a computational treatment guideline.

It is the responsibility of NBA Engine 71 (or whichever software module is traversing the guideline) to map the current patient state to the correct location in the computational guideline by inferring that RA has already been diagnosed and it is time to begin the treatment. Assuming that rheumatoid arthritis has been positively diagnosed, then control may begin at node 550 of FIG. 6B.

As shown, condition 502 may have links 503a to any number of EDFs, links 503b to any number of EDIs, links 503c to any number of other differential conditions and links to ETIs. A first decision node 504 decides which guideline 506-512 should be used for diagnosing the condition. Guidelines are typically published by a medical association such as the American Heart Association, the American College of Cardiology, or any number of other associations, governments or corporations such as pharmaceutical companies, healthcare systems, pharmacy benefit managers, etc. Each guideline may have a different approach to diagnosing and treating a particular condition, such as the order in which tests are performed, the type or name of these tests, which drugs are prescribed first, drug dosages, first line treatment, second line treatment, or forking the treatment on the basis of demographics for appropriate conditions. It is also possible that a particular guideline is a modification of, or a combination of, an established guideline from any medical association. A guideline may begin with diagnosis and lead into treatment (in this example), may be diagnosis only, may be treatment only, or may perform diagnosis by suggesting treatments. As there may be any number of guidelines below such a decision node 504, the decision may be to choose a particular diagnosis guideline if the condition is suspected or unknown (as in this example), to choose a treatment guideline if the condition is known, or perhaps to choose a treatment guideline if the condition is suspected (diagnosis by treatment). The clinician may be allowed to choose a particular guideline, the system may default to always choose a guideline from the AHA, for example, or other criteria may be used. To make it easier for clinicians, the system may pick a guideline from a default source. A clinician also has the option to select a guideline from their preferred source via clinical co-pilot 24.

Assuming that the decision is to follow the guideline at node 506, control then moves to a decision node 514 which links to an EDI representing a query such as “patient has swollen joints?” and “how many swollen joints?” and “for how long has the patient had these swollen joints?” Typically, such direct observation or questions can also be coded as investigations or EDIs. Hence, observation decision nodes as well as lab decision nodes link to EDIs while their respective results link to EDFs and EDI results. Depending upon the use case, the information can be taken from the EHR or the clinician is prompted to ask the patient. When a laboratory test should be performed or a diagnostic factor needed, the guideline attempts to deduce results for all decision nodes given the patient state even if the result is not explicitly mentioned. But, if the guideline fails to do so and arrives at a node with no way of pursuing a particular child node, it will prompt the clinician on a user interface.

Typically, the set of all factors needed to interpret a decision result node is represented as a single EDF node. Also, the NBA Engine also uses inferential reasoning (e.g., using the ontology link in an EDF) to infer additional information about the patient based upon the patient state to expand the factors that the patient has, in order to provide answers to as many decision nodes as possible. Matching the patient data to the EDF “five swollen joints” will typically take into account demographics from a demographics node linked to that EDF. Preferably, guideline component nodes link to “must have” factors by default. Further, this EDF also links to the condition “rheumatoid arthritis” as it is a diagnostic factor for that condition.

There may be any number of decision result nodes below a decision node such as 514, and assuming that the result is three swollen joints or less, then control moves to node 516 and then on to determination node 518 which determines that rheumatoid arthritis is not present in this patient. The determination node acts as a stopping point for the flow that is traversing the guideline and the way it manifests itself will change from use case to use case. For instance, if a clinician is using the platform the guideline may end with a response suggesting that treatment is complete. If software is traversing through the guideline, the determination acts as a conditional statement indicating the end of treatment has been reached and hence the execution of treatment (or at least a single path of treatment) should end.

Assuming that the result is five swollen and tender joints for more than three weeks 520, then rheumatoid arthritis is still possible and control moves to decision node 522 to perform a test. Node 522 links to an EDI which represents a laboratory test for rheumatoid arthritis. In general, a decision node may link to any number of EDIs as there may be more than one way to represent a particular diagnostic factor or investigation. For example, there may be more than one blood test used to test for rheumatoid factors, depending upon the test used by a particular hospital, the particular machine used, etc. Preferably, a decision node links to EDIs because a slight variation in tests will result in a different unique identifier for each test.

There may be three possible results from this test shown in decision result nodes 524-528. If the result is negative then control moves to node 518 as rheumatoid arthritis is absent. If the result is extremely positive then control moves to node 538 and a determination node indicating that rheumatoid arthritis is diagnosed. If the result is positive in node 526 then a determination cannot be made either way and control moves on to decision node 530 for another laboratory test to confirm or rule out rheumatoid arthritis.

In this example, the flow continues and decision node 530 is linked to one or more EDIs that provide the results of a protein test. Again, if the test result is negative then control moves to node 518 and a determination of no rheumatoid arthritis, if the result is extremely the positive then control moves to node 538 and rheumatoid arthritis is diagnosed, while if the result is positive then control moves to decision node 540 to perform another test. The ESR node 540 links to any number of EDIs which may return a result of normal or elevated depending upon the results. If normal, then there is a determination of no rheumatoid arthritis, while if elevated, then yet one more laboratory test is performed at decision node 546. Decision node 546 links to any number of EDIs which, after reference to the patient's EHR, obtaining results from a new test or a question for the patient, a result of normal or elevated is returned. If normal at decision result node 548 then there is a determination of no rheumatoid arthritis, while if elevated, then control moves to node 538 for a determination of rheumatoid arthritis.

In this fashion, the computational guideline facilitates implementation of a flow that may have a series of laboratory tests, examinations or questions regarding diagnostic and risk factors, each result perhaps confirming or ruling out a suspected diagnosis, or leading to further investigations, or requests for patient diagnostic and risk factors. In this example, a guideline has been made computational and a diagnosis has been reached based upon the results of laboratory tests, examinations, and diagnostic and risk factors obtained from the patient, from the patient's EHR or from observations by the clinician. This flow may begin with a suspicion of a condition and no other information available, in which case the flow prompts for investigations to be performed, factors to be asked about, etc. Or, certain laboratory tests or examinations may have already been performed, and the patient's EHR may contain various known diagnostic factors and risk factors, but at any point within this flow there may be laboratory tests or examinations that need to be performed and factors that need to be asked about, in which case the flow prompts for those results. In a typical scenario, a clinician begins with questions about diagnostic and risk factors because those are simple, quick and inexpensive, and then moves on to any needed laboratory tests and examinations as those are more expensive and require more time but can be more definitive.

Assuming that a determination has been reached in node 538 then control moves to node 550 which then implements a computational guideline for treating rheumatoid arthritis (based upon the original guideline chosen in node 506). Similar to FIG. 5A, the treatment guideline of FIG. 6B essentially begins at determination node 538 in which a particular condition has been diagnosed. For example, if RA has already been diagnosed and confirmed, the system may quickly traverse from node 502 down to node 538 if all of the relevant diagnostic factors are known and the relevant laboratory tests have been performed; thus, treatment begins more or less immediately from the clinician's perspective. Alternatively, the flow may begin at a condition node which represents the condition rheumatoid arthritis. It is the responsibility of the NBA Engine traversing the treatment guideline to find the location of the patient in the treatment guideline by analyzing the patient state. If the clinician knows (via the EHR) that the patient has rheumatoid arthritis, he or she may jump directly into treatment using the manual or automated methods described above. Decision node 550 has a link or links to one or more EDIs, each representing an investigation termed a Clinical Disease Activity Index (CDAI), again, each EDI representing a different implementation of that investigation, if available. Multiple variations of a test to do accomplish the same tasks can exist. All of these variations will have their unique identifier code which is picked up from the EHR or is assigned by the NLP engine if the concept is extracted from unstructured notes. The engine looks up the correct EDI in the graph using this code.

The possible results from this investigation are remission, low activity, moderate activity and high activity, shown in decision result nodes 552-558. If the result is remission, then control moves to the determination node that no treatment is needed and the flow comes to a stop. If the result is low activity, then control moves to a therapy choices node 562 representing hydroxychloroquine as a therapy in this instance. In this example, there are not multiple options for different classes of this drug and control moves to therapy ingredient node 564 which recommends a prescription for the drug hydro chloroquine (HCL). The dosage regimen is calculated in dosage decision node 565 (using its rule link) and stored within that node. This dosage regimen from node 565 is then recommended to the clinician. The recommendation may take the form of an output which is displayed on a computer device to the clinician, an output of the recommendation in electronic form, or any other suitable option for outputting the recommended prescription. The clinician thus prescribes the drug, the patient takes the drug and then the patient is monitored over time. The act of prescribing or taking the drug and entering that into the platform depends on the use case. If claims data is being analyzed, that act will already be presented in a claim. If not, the clinician will update the patient state by entering the information in their EHR with which the platform is integrated. Or, there is an option for the clinician to enter this information manually using a user interface.

Decision node 567 implements a decision delay in which after a certain delay or delays more tests may be performed. Data may be input indicating how much time has passed and may be input from the EHR, patient, clinician or other. To determine that the relevant time period has passed, the NBA Engine keeps track of a reference time remaining for executing a particular node. The first time the NBA Engine visits a delay decision node it sets a timer for all possible events (two in this case) to zero indicating that the timer has begun for both nodes. Then, every time a path is visited the engine resets the delay corresponding to the last occurrence of that event to zero and indicates a restart for the timer for that event. The delay is not reset for the path that was not visited, ensuring that the time to event is being calculated from the visit when the timer was last set to zero.

If the result is that three to six months have passed then control moves to node 568 and this result indicates that a blood test should be performed by using the one or more links to an EDI, whose results may be moderate, low or high levels of HCL in the patient's blood as shown in nodes 574-578. Basically, if the level is moderate then the dosage is kept constant as shown in drug adjustment node 580, if the level is low then the dosage is increased in node 582 and if the level is high then the dosage is decreased as shown in node 584. The drug may be adjusted using a factor, i.e., a factor of “1” means keep the drug dosage constant. The factor within each of the drug adjustment nodes is dependent upon the particular drug, the condition, a particular guideline and other indication such as demographics, laboratory results, therapeutic response to earlier dosages, and other elements from the patient state. The clinician thus writes a new prescription for the patient depending upon nodes 580-584, the patient takes the drug and control returns to node 564.

As each of these three nodes 580-584 lead back to node 564, here is an example where an edge may carry the property representing the factor rather than implementing it explicitly using drug adjustment nodes 580-584. By way of example, each of the edges leading from nodes 574-578 may lead directly to node 564 and each of those edges carries the relevant factor, such as a factor of “1”, a factor greater than one and a factor less than one, respectively. Thus, nodes 580-584 do not exist and the factor of each of those nodes is simply a property of their respective edges leading to node 564. Where suitable, and depending upon a particular implementation, storing properties on edges instead of in explicit nodes may be used anywhere within the knowledge graph.

Once control returns to node 564 every three to five months this blood test and possible adjustment will be repeated. Once the decision result from node 566 indicates that five years have passed, then control moves to an ophthalmology test in decision node 586 which may use any of a number of ophthalmology tests indicated by a link to one more EDIs. If the result is that the patient does not have retinopathy, then control moves to decision result node 590 and then on to drug adjustment node 580 indicating that the drug level should be kept constant. If, however, the result is that the patient does have retinopathy, then control moves to node 588 and then on to a new therapy choices node 560 in which a new drug (a second line treatment) may be prescribed due to the side effects of HCL and use of HCL is stopped.

Therapy choices node 560 recommends the drug methotrexate, and although not shown in this diagram, the flow continues from node 560 in order to choose a particular therapy ingredient based upon methotrexate, a decision delay node follows, tests at particular frequencies monitor for the drug in the patient's blood and for adverse reactions, all in a similar manner as shown in nodes 564-590 for the drug HCL. With RA, patients do not fully recover; they undergo treatment to keep it under control. Hence, this flow never ends but based upon a change in his or her biomarker the treatment may continue to be updated via drug dosage adjustment, drug change etc. With other flows, a determination node may mark the end.

Above, FIGS. 5A, 5B, 6A and 6B show portions of computational guidelines in which a drug is eventually prescribed in order to treat a particular condition. A treatment guideline may also include nodes representing decisions, results, delays and determinations that represents a computational guideline for performing a therapy that does not necessarily include drugs. By way of example, node 406 is the beginning of a guideline for a different therapy that may not include a drug; any of the nodes 504, 508 or 512 may also be the beginning of a guideline that implements a therapy not including a drug, and once a condition is diagnosed (such as in node 538), control may branch to a decision node (not shown) that decides whether to implement a therapy involving medication (such as node 550) or to a different set of nodes which implemented therapy not involving medication. Of course, any particular treatment guideline may include a mix of medication and non-medication therapies. Accordingly, a therapy guideline beginning at node 406 (for example) may include any number of decision nodes (with corresponding decision result nodes having links to EDFs or to EDIs), any number of decision delay nodes (with their corresponding decision result nodes), and any number of determination nodes, all arranged to represent a computational guideline that may or may not include therapy choices nodes, therapy ingredient nodes, drug adjustment nodes, or other nodes related to drugs.

### Example Computational Nodes

Any of the nodes described herein (whether in the clinical knowledge graph or in a computational guideline) include a unique identifier within the comprehensive graph such as a hash value (over any relevant information of the node), a universally unique identifier (UUID), a unique name, or other suitable unique identifier. After any traversal of a guideline for a patient (whether for diagnosis, treatment, etc.), a representation of that traversal is stored as a subgraph in patient state service 60 and records those nodes that were traversed. Any suitable representation may be used such as: a list of nodes traversed in order by their unique identifiers, a list of nodes traversed by their unique names, pairs of nodes stored together indicating a traversal from one to the other, a graph-based representation of the nodes traversed, etc. Such a subgraph need not include all of the details of the nodes described above and below, only an identifier identifying which node in the comprehensive graph is needed. Further, if a guideline references an EDF, EDI or ETI, such a reference or an identifier of one of these elemental nodes need not be included in the subgraph as such a reference or identifier can be accessed via one of the computational nodes. Thus, patient state service 60 includes a subgraph for every traversal for a patient and each subgraph may be indexed by a unique patient identifier for later retrieval of that subgraph. During a later query or analysis, a particular patient subgraph may be mapped to a computational guideline in the comprehensive graph to understand that patient's path and elicit more information.

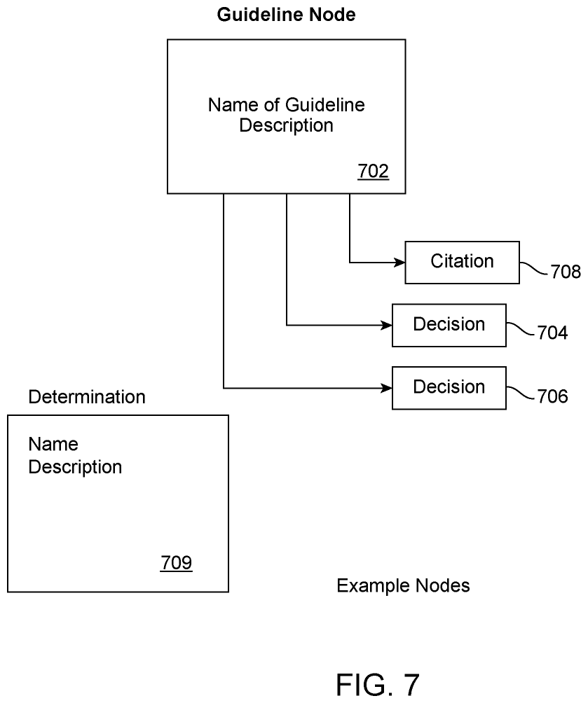

FIG. 7 is an example of a guideline node 702 and a determination node 709. While a guideline node represents the beginning of a computational guideline, the determination node represents a possible end of that guideline. As mentioned earlier, a guideline node may represent a guideline from a particular publisher regarding how to diagnose a particular condition, how to treat a particular condition, or a combination of both. The node includes the name of a particular guideline (e.g., Cardiology Association) and a description indicating what the guideline diagnoses and treats, and other related information. This node may be linked to any number of decision nodes 704, 706 described in more detail in FIG. 8, and especially to observation decision nodes, allergy decision nodes or laboratory decision nodes. The node includes a link (or URL) to a citation 708 which is an online article, paper, a reference to a location within a published medical journal, database or similar, or a reference to a Web page or Web site. An example is the following citation standard (inspired from AMA) that includes the name of authors, title of publication being cited, publication name, date, volume or issue, page numbers, and DOI. The citation would be: Authors: Smith J, Jones M; Title: Clinical outcomes of XYZ; Publication name: JAMA; Publication date: 2023; Volume/Issue: 329 (10); Page Numbers: 891-901; DOI (digital object identifier): 10.1001/jama.2024.24276.

Determination node 709 includes a name (e.g., “rheumatoid arthritis diagnosed”) and a description indicating what this determination means (i.e., a description as to why the condition is diagnosed or not, why the treatment is successful or not, etc.). It typically is not linked to other nodes as it determines the end of treatment flow.

FIG. 8 shows examples of decision nodes. Other types of decision nodes may also exist depending upon the specific implementation of the knowledge graph and its intended use. In general, a decision node will have a link to another node in the knowledge graph (e.g., an EDF or EDI) or to an outside source (e.g., input from the clinician on a computer) in order to obtain information to make its decision, and a link to a result node or nodes (e.g., a guideline node, a decision result node) depending upon the obtained information.

Shown is a guideline decision node 710 having a name, e.g., “guidelines for rheumatoid arthritis” and a description of the available guidelines and how and why one may be chosen over the others. Accordingly, the node includes links to any number of guidelines 702 and a link to a citation 712 describing why these multiple guidelines are relevant for a particular condition. Shown also is an observation decision node 716 having a name such as “number of swollen joints” and a description indicating how this decision relates to a diagnosis or treatment of the guideline. An ontology link links to a medical ontology such as SNOMED-CT and specifically to the swollen joints concept in that ontology. A rule link provides a link between this decision node and rules indicating which of the possible results should be followed. The node has any number of links to observation decision result nodes 744 and any number of links to EDIs 718. There are links to any number of EDIs because there can be variations of an investigation for performing the same task (for example, investigation of bacteria presence using a stool sample versus a urine sample). The term “observation” is used to refer to factors of the patient observed by the clinician, and thus links to an EDI or EDIs in order to make a decision and obtain results by querying the EHR, by asking the patient, by a clinician's observation, by an investigation, or by other technique.

By linking to an EDI we can (a) reason using a SNOMED hierarchy to infer which other factors the patient has, and (b) reason which other conditions the patient might have and hence which signs and symptoms related to that condition the patient may potentially have. The EHR might suggest a patient has two or three factors, but once we map all of the patient state EDIs we are able to infer more factors than present in the EHR.

Shown also is an allergy decision node 722 having a name such as “penicillin allergy?” and a description indicating how this decision relates to a diagnosis or treatment of the guideline. An ontology link links to a medical ontology such as SNOMED and specifically to the penicillin allergy concept in that ontology. The node has any number of links to allergy decision result nodes 750 and any number of links to EDIs 724. Typically, an allergy decision node may link to any number of EDIs because for particular class of drug, such as penicillin, there may be numerous types of penicillin available and thus possible allergies to each of those types; each EDI represents a possible allergy to each type.

Shown also is a laboratory decision node 726 having a name such as “penicillin susceptibility?” and a description indicating how this decision relates to a diagnosis or treatment of the guideline. An ontology link links to a medical ontology such as SNOMED and specifically to the penicillin susceptibility concept in that ontology. The node has any number of links to laboratory decision result nodes 756 and any number of links to EDIs 728. Typically, a laboratory decision node may link to any number of EDIs because a particular laboratory test may be implemented in a different way depending upon the hospital, may have a different name, or may use a different machine with a different range. Optionally, each lab decision node may include possible result types such as numeric scaler, nominal, binary, ordinal, a numeric range, etc., in order to enforce which result types are possible for particular test. These result types may or may not be used depending upon the implementation. A laboratory decision node represents any laboratory test or examination, i.e., any investigations that might be performed. Laboratory decision nodes, observation decision nodes, and allergy decision nodes may also link to the LOINC ontology as well.

Shown also is a delay decision node 732 having a name such as “wait three months?” and a description indicating why wait and how this waiting period relates to a diagnosis or treatment of the guideline. The node has any number of links to delay decision result nodes 762.

Shown also is a dosage decision node 736 having a name, description, a dosage regimen and a rule link that links to a rule for calculation of the dosage regimen for a given patient after the drug has been selected. For a given drug, there are a set of rules to determine the strength, frequency, route, and duration of the prescription. Further, a patient's clinical condition can also dictate change in this dosage regimen. These rules are stored in any suitable database and may be dynamically updated while traversing the guideline (for example, if the patient state does not improve after a medication, a rule may increase the dosage volume). The rule link refers to such links between a drug (via its therapy ingredient node, e.g., node 564) and the rules wherever they are maintained. The dosage regimen is stored within the dosage decision node and includes: the units for a drug (e.g., “milligrams”), the frequency with which it should be taken, the strength of the drug, route, a link to the drug description in an ontology, a SIG link which is a link to the actual prescription information (e.g., “take two tablets twice a day for 14 days”) and its form. Node 736 links to a decision node 738 such as a delay decision or an observation decision. We can adjust for any kind of delay by changing “quantum” in the duration.

As shown, many of the decision nodes include a rule link to a database of rules for reasoning. For a particular decision node, in this database there are node-specific rules on how each particular decision node should be reasoned in order to select an appropriate child node for traversal. By way of example, for lab decision nodes the rules dictate how to compare the patient laboratory results with the lab decision results available below the lab decision node. For delay decision nodes the rules describe how to track reference wait time before an event occurs and then traversing to the correct child node when the wait time is up. For observation decision and allergy decision nodes, the rules describe how to compare the patient results with the child decision results to choose which path to traverse. These rules are stored in an appropriate database and are looked up by the system as it traverses the computational guideline.

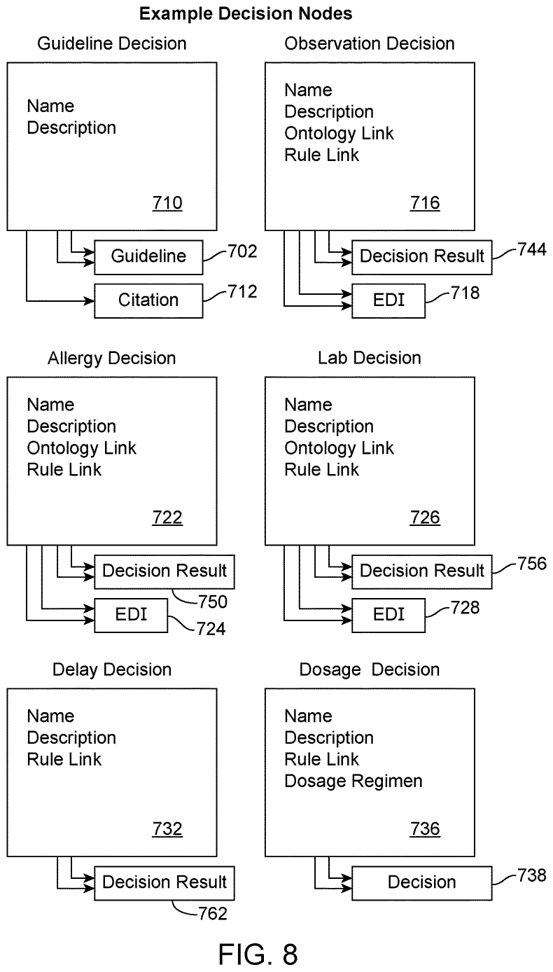

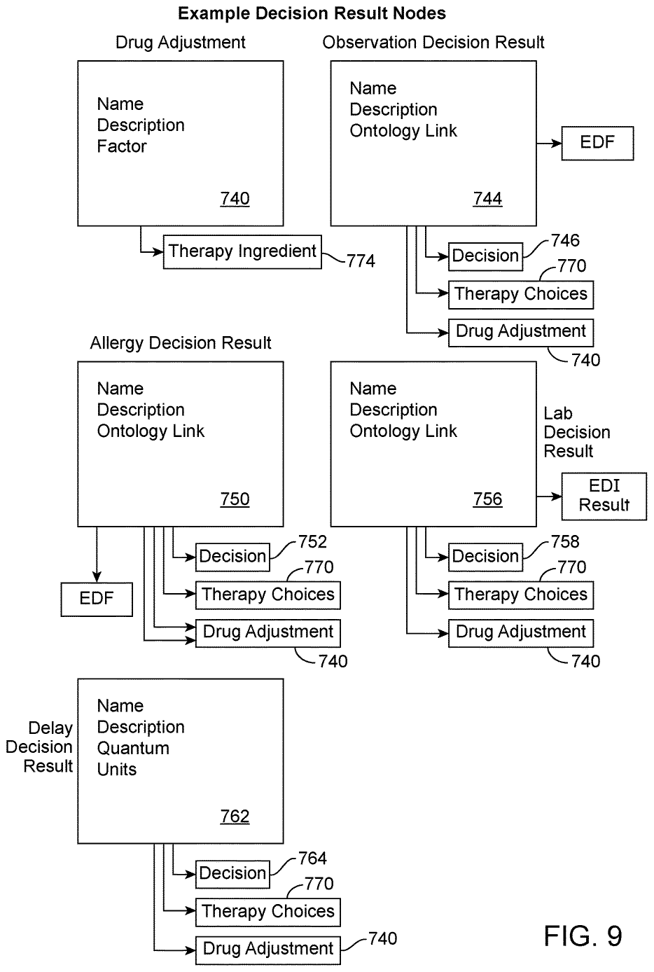

FIG. 9 shows a drug adjustment node along with various decision result nodes. Drug adjustment node 740 includes a name (e.g., constant, increase, decrease) and a description indicating how the dosage of a particular drug is adjusted and why, in light of the results from a laboratory test, observation, etc. A factor indicates how a current dosage of a drug for the patient should be adjusted, and may use numerals (e.g., “1” means keep constant, “0.5” means decrease by half, “1.5” means increase by one and a half times), may use percentages (“80%” means decrease to 80% of the current dosage, etc.), or may use any other indication to recommend that a drug dosage should be decreased, kept constant, increased or eliminated. This node links to a therapy ingredient node 774.

Any number of decision result nodes may be used and generally each corresponds to a particular decision node, such as those previously described. Shown is an observation decision result node 744 including a name and a description, a link to a concept in an ontology representing the particular observation result (e.g., “at most three swollen joints”). Allergy decision result node 750 includes a name (e.g., “severe allergy”) a description and a link to the concept of a particular allergy result to a particular drug in a medical ontology. Lab decision result node 756 includes a name (e.g., “low level”) a description, and a link to a concept in an ontology representing a particular laboratory result (e.g., “resistant to penicillin”). Delay decision result node 762 includes a name (e.g., “a delay of five years”), a description, a quantum (e.g. “5”), and units (e.g., “years”). These decision result nodes 744, 750, 756 and 762 may link to any decision node, a therapy choices node 770 or a drug adjustment node 740, and preferably link to only one of these in a computational guideline. Nodes 744 and 750 link to EDFs while node 756 links to EDI results.

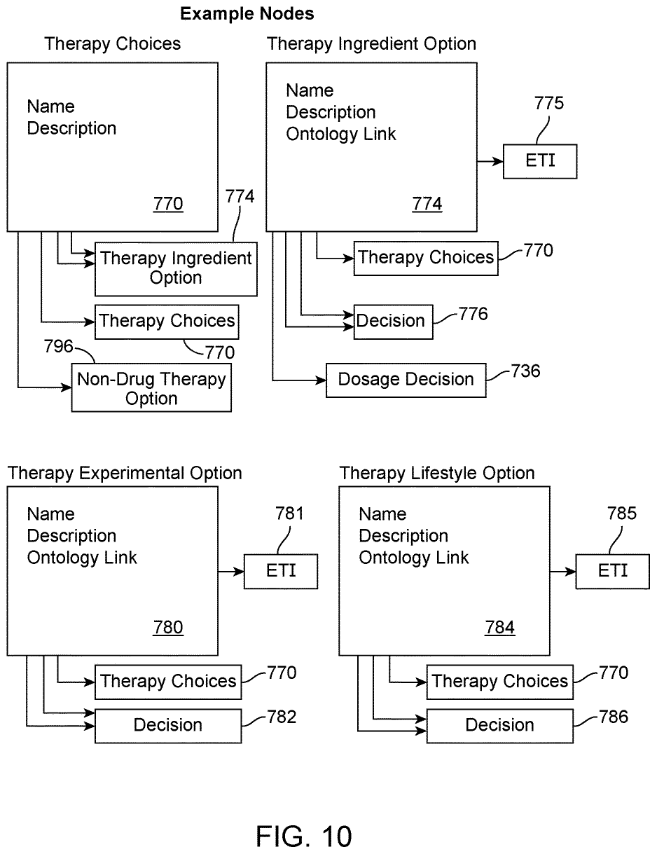

FIG. 10 shows examples of a therapy choices node, a therapy ingredient options node, and two nondrug therapy nodes. Therapy choices node 770 includes in the case of a drug choice, a name (e.g., “penicillin”), a description of the drug, including its class, the various choices in the class, how it is used to treat a particular condition, etc. In the case of a nondrug therapy, included is name and description of the nondrug therapy. This node has links to any number of therapy ingredient option nodes 774 or to a single other therapy choices node 770. Also included is a link to any of the nondrug therapy option nodes 780, 784, 790-794 or 796 described below.

Therapy ingredient option node 774 includes a name (e.g. “penicillin G”), a description indicating the particular ingredient and how it may be used, and a link to this particular drug ingredient in an ontology such as RxNorm or other ontology. Node 774 may link to a therapy choices node 770 or to any number of decision nodes 776. Because a drug might be presented in a myriad of ways (e.g., NDC codes, RxNorm codes for related SCDs, etc.), a therapy ingredient option node 774 also includes an ontology link to RxNorm (for example) which is used to map the drug indicated (and prescribed to a patient) to a standardized code which acts as a single source of truth in a knowledge graph and represents other drugs comprising of the same ingredients.

In addition to the therapy ingredient option nodes which each provide an ingredient of a drug for treating a particular condition, any number of nondrug therapies may be represented by other therapy nodes within a guideline for treating a condition in order to recommend or prescribe such a nondrug therapy. In the same way that a therapy choices node 770 may link to a particular therapy ingredient option node 774, therapy choices node 770 may also link to any nondrug therapy option such as node 784 to provide a treatment not involving a drug. Shown is a therapy lifestyle option node 784 having a name, description, and an ontology link; this node requires a change in lifestyle such as in nutrition, exercise, sleep, etc. A therapy experimental option node 780 also has a name, description and an ontology link; it includes any emerging therapy such as regenerative medicine, nanotechnology-based therapies, etc.

FIG. 11 shows further nondrug therapy nodes. Therapy procedural option node 790 includes procedures such as surgery, radiation therapy, chemotherapy, cryotherapy, etc. Therapy rehabilitation option node 791 includes speech therapies, occupational therapies, cardiac rehabilitation, neurological rehabilitation, etc. Therapy psychological option node 792 includes behavioral and psychological therapy such as cognitive behavioral therapy, dialectic behavioral therapy, group therapy, etc. Therapy complementary option node 793 includes alternative therapies such as acupuncture, aromatherapy, ayurvedic therapy, etc. Therapy genetic option node 794 includes gene therapy, stem cell therapy, etc.

Although these nondrug therapies are shown represented each by its own node, in an alternative embodiment a single generic therapy nondrug option node 796 may represent any of these nondrug therapies. Node 796 includes a “tag” field which describes the type of nondrug therapy represented such as any of therapies 780, 784 or 790-794, e.g., the tag may be “Therapy Lifestyle.” This alternative representation reduces redundancy, although if a particular nondrug therapy links to a particular calculation or dosage decision then individual nodes may be used. Each of these nondrug therapy nodes includes a link or links to therapy choices or decision nodes as shown, and each includes a link to an ontology from SNOMEDCT, CPT, FDB, etc.

Similar to how a therapy choices node may lead to any number of therapy ingredient option nodes (e.g., nodes 428-434 of FIG. 5A), a therapy choices node for a nondrug therapy may lead to any number of nondrug therapy option nodes such as therapy experimental option, therapy rehabilitation option, etc. By way of example, a CBT therapy choices node (a type of psychological therapy) may have child nodes such as exposure, DBT, REBT, which are all therapy psychological option nodes. And the exposure node may link to an ETI virtual reality exposure. In another example, a heart surgery therapy choices node (a type of procedural therapy) may have child nodes such as coronary artery bypass grafting surgery, heart transplantation surgery, etc., which are both therapy procedural option nodes.

As shown, various of the computational guideline nodes in FIGS. 10 and 11 may link to an elemental therapeutic intervention (ETI) node (described below) in order to assist with execution of the guideline before returning to the flow of the guideline.

### Elemental Therapeutic Interventions

An ETI (elemental therapeutic intervention) is similar to the EDFs and EDIs described herein in that certain therapy choices, for example, a therapy ingredient option (i.e., a drug) or a nondrug therapy (lifestyle, rehabilitation, etc.) in a computational guideline link to ETIs in a clinical knowledge graph, just as decision nodes and decision result nodes link to EDIs and EDFs. A therapy choice may be a pharmacological or drug-based intervention, a surgical or procedural intervention, a physical therapy or rehabilitation intervention, a psychological or behavioral intervention, a lifestyle or preventative intervention, or an alternative therapy or a complementary therapy. These drug and nondrug therapy choices are described in FIGS. 10 and 11 above. An ETI represents the basic, atomic (or “elemental”) therapeutic intervention that is applicable across conditions. For a drug, the ETI represents the actual prescribable drug, e.g., while therapy ingredient option node 434 represents the penicillin V ingredient in general, the ETI to which node 434 links represents the actual prescribable penicillin V drug for the patient. A therapy option node in a guideline may link to one or more of these ETIs.

Similarly, an ETI for a nondrug therapy also represents the actual prescribable therapy for the patient. By way of example, a node “CBT therapy choices” (representing possible cognitive behavioral therapy options) may have three therapy psychological option child nodes such as exposure therapy, dialectical behavior therapy (DBT), rational emotive behavioral therapy (REBT) and others. Each of these child nodes represents a particular CBT therapy but does not represent the actual prescribable therapy that a clinician would order. In other words, a clinician does not simply prescribe “give the patient exposure therapy,” he or she is more specific. Thus, multiple ETIs may exist for particular, prescribable types of exposure therapy such as imaginal exposure therapy ETI, in-vivo therapy ETI, virtual reality exposure therapy ETI, etc., each possibly including frequency, duration and other specifics depending upon the type of therapy. Accordingly, the child node exposure therapy links to each of these ETIs. A “prescribable” therapy represented in an ETI (whether a drug or nondrug therapy) thus represents a specific recommendation or prescription that a clinician gives to a patient for a particular therapy and its specifics will depend upon the particular therapy.

FIG. 12 illustrates a portion of a clinical knowledge graph and an abbreviated computational guideline in which nodes in a computational guideline are linked to ETIs. In general, portions of a knowledge graph including nodes such as those shown in FIGS. 2-4 and 13 may be referred to the clinical knowledge portion of the knowledge graph, while those portions shown in FIGS. 5A-6B and including the various types of nodes shown in FIGS. 7-11 used in executing a computational guideline may be referred to as a computational guideline.

Shown is a condition 602 (for example, the acute strep node 402 from FIG. 5A) that links to a therapy group 604, which itself links to any number of therapy restriction nodes 606, 608. The therapy group node 604 has a type such as “drug,” and the name of a group of drugs that it represents such as “β-lactams.” Therapy restriction node 606 includes one or more criteria indicating why the therapy represented by ETI 610 or 612 should not be used, such as an allergy or a drug-drug interaction, and may connect to any number of ETIs. Node 608 connects to ETI 614 representing the prescribable drug amoxicillin.

A particular ETI such as 610 may be linked (directly or indirectly) to any number of conditions; as shown, condition 620 is linked to therapy group 604 or may be linked to ETI 610 via a different therapy group 622 and different therapy restrictions 624. For example, while therapy group 604 are “β-lactams,” therapy group 622 may be “antibiotics.” In this fashion, an ETI is not specific to a particular condition, does not include condition-dependent information and may be used across conditions. Condition 602 also links directly to ETI 636 which is a particular photodynamic therapy that may be used with acute strep.

Also shown is a small portion of a computational guideline, nodes 630, 434, 632 and 634 (the dashed lines indicating other computational nodes of the guideline not shown). Guideline node 630 may represent a guideline for acute strep of FIG. 5A or for a different condition. Note that for any guideline for any condition that suggests use of penicillin V to treat a condition in tablet form, there will be a link to ETI 610, thus each ETI is independent of conditions and may be used across conditions. Any therapy ingredient option node such as 434 may be linked to an ETI, thus making available the actual prescribable drug to the computational guideline. Any nondrug therapy node in a computational guideline, such as those shown and described in FIGS. 10 and 11 may also link to its corresponding ETI in the clinical knowledge graph. For example, assuming that guideline 630 recommends a light-based therapy for acute strep at some point via node 632, shown is a photodynamic therapy child node 634 which is one of any number of therapy procedural option nodes (link 633 may link to other such option nodes). Node 634 thus represents the photodynamic therapy option in general and it links to an ETI 636 which is the specific photodynamic therapy intervention that may be prescribed (i.e., including duration, color of light, photosensitizer chemical, etc.). ETI node 636 may be linked via 637 to other condition nodes representing conditions that also benefit from use of ETI 636 (via a therapy restriction node and a therapy group node or directly as discussed above).

As shown, a node in the computational guideline (e.g., node 434 or 634) links to, or references, an ETI node which is not in the computational flow. In other words, even though an ETI node (or an EDF or EDI node) is referenced by the computational guideline, the flow returns to the computational guideline after traversing to or referencing the ETI node in order to continue with the guideline. In one example, any node of a computational guideline may reference an ETI, EDF or EDI node in order to obtain its ontology link and then follow that link electronically to the corresponding remote ontology to perform inferencing or reasoning (e.g., by traversing up or down within that ontology) or to obtain information from the ontology via that link, and then to return electronically to the computational guideline with the result obtained from the ontology. Thus, a referenced EDF, EDI or ETI node may be used in different manners.

If a therapy ingredient option node or a nondrug therapy option node links to an ETI, the flow of a computational guideline may traverse to the ETI to recommend the specific drug or nondrug therapy, may traverse to the ETI for reference or information, or may not traverse to the ETI, depending upon the use case. For instance, the flow may traverse to the ETI if the guideline needs to find more information not available within the computational guideline itself, e.g., “to which other conditions is this therapy ingredient option applicable?” In this case, the flow traverses to the ETI and then may traverse upward through the knowledge graph to find all linked conditions. This traversal helps improve differential diagnosis being executed by the NBA Engine if needed. A traversal to an ETI from a therapy ingredient option node (or from a non-drug therapy option node) may also be used if more information is needed than what is found in the therapy node; any information in the linked ETI may be returned to the guideline. A traversal to the ETI is not needed in those use cases where the computational guideline or patient state service has sufficient information.

Therapy restrictions (e.g., in node 606) may be used during the guidelines according to different use cases. In general, restrictions inform when a particular therapy of an ETI should not be prescribed, recommended or given, for example, to which demographics should not be given a particular therapy. In one particular use case, the node helps to detect whether a patient had been mistreated by any prescribed therapy; the criteria in any therapy restriction node corresponding to an ETI prescribed for the patient can be used to determine whether any restriction has been violated.

As shown, even though a particular ingredient option (for example, 434) links to its ETI 610, it is not always necessary to traverse to that ETI when prescribing a drug in the course of the guideline as the dosage decision node typically has all required information for the clinician. Similarly, in the case of a nondrug therapy (for example, node 634), the therapy option node provides enough information for the clinician to make a recommendation or prescription; he or she can prescribe the therapy using information from the therapy option node in the guideline, leaving some of the actual details up to him or her.

Even though a guideline may perform a decision (e.g., check for an allergy in node 408) or recommend a prescription, links to ETIs and to their parent restriction and group nodes allow any flow traversing a guideline to find more information than is available in the guideline alone. By way of example, during treatment for condition 602 at 434, the flow may consult nodes 610, 606 and 604 to obtain more information on penicillin V and its restrictions. In another example, assuming that a prior traversal of guideline 630 had been performed for a patient and saved in patient state service 60, this prior traversal may be retrieved and consulted when treating the patient again. Knowing that the patient had taken penicillin V (node 434), traversal to node 604 reveals that the therapy group is anti-bacterial and lets the backend engine know that this patient had a bacterial infection.

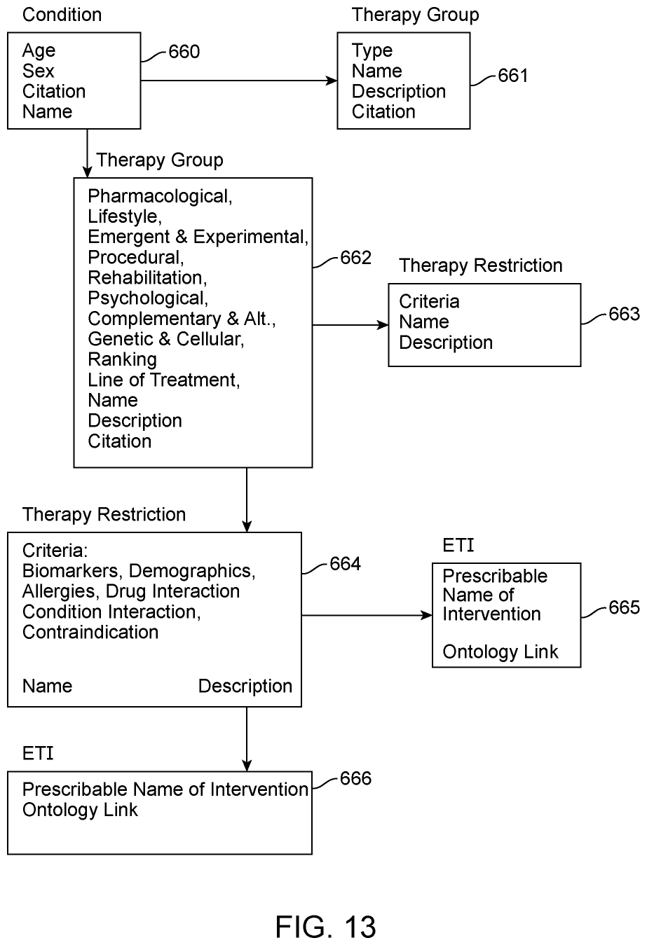

FIG. 13 is a block diagram describing in more detail the nodes of FIG. 12. As shown, any condition 660 of a clinical knowledge graph may link to any number of therapy group nodes 661, 662. A therapy group is a high-level aggregation of similar therapies. For example, in the case of drug-based therapies, a therapy group may be constituted on the basis of the pharmacological properties of drugs (e.g. on the basis of mechanism of actions, such as beta blockers), on their chemical structure, or on their therapeutic effect (antibiotics versus anticoagulants). In the case of psychological therapies, a therapy group may represent a high-level definition of psychological or behavioral therapies such as CBT, DBT, etc.

A therapy group node includes the properties of type, name, description and a citation link in a medical evidence source providing support for grouping particular therapies by type. The type property indicates the type of therapeutic intervention that is grouped below the therapy group node, i.e., any number of ETIs 663, 665, 666. The type of therapeutic interventions may be pharmacological or nondrug interventions such as lifestyle (e.g. preventative, nutrition, exercise, sleep), procedural (e.g., surgery, radiation, chemotherapy, cryotherapy), rehabilitation (e.g. speech therapies, occupational therapies, cardiac rehabilitation, neurological rehabilitation), psychological (e.g. cognitive behavioral therapy, dialectic behavioral therapy, group therapy, exposure, DBT, REBT), complementary & alternative (e.g. acupuncture, aromatherapy, ayurvedic therapy), genetic & cellular (e.g. gene therapy, stem cell therapy), or emergent & experimental (e.g. regenerative medicine, nanotechnology-based therapies). Further, any of the nondrug interventions may be represented by a nondrug therapy type, all of which have been described above in FIGS. 10 and 11.

A therapy group node holds condition-dependent properties including a ranking (e.g., a number mapping to usage probability of a drug for its linked condition). In general, the ranking indicates that the ETI is used X percent of the time as per the clinician's judgment to manage the linked medical condition. For example, penicillin G benzathine has a ranking of 1 for the condition syphilis, indicating that for 90%+ of syphilis cases, it is the drug that is prescribed. A ranking of 2 means that the ETI is used to treat the condition 30-90% of the time and if a 3 the ETI is used to treat the condition less than 30% of the time. Preferably, the ranking values for factor groups and investigation groups use similar numerals and scale, i.e., a 1 meaning highly indicative that the patient has the linked medical condition, while a 3 means less of a likelihood that the patient has a condition. The ranking scale used for all three groups may be inverted (i.e., 3, 2, 1 instead of 1, 2, 3), meaning that a highest final ranking value means the most likely condition. Also included is a line of treatment, i.e., whether the drugs in the group are first line, second line, etc.

Therapy group node 662 may be linked to any number of therapy restriction nodes 663, 664, and each therapy restriction node includes one or more criterion indicating why a therapeutic intervention (i.e., an ETI) linked to the therapy restriction node should not be used. These criteria can include: condition interactions (e.g., patients with cardiac issues should not be recommended certain exercises); patient biomarkers (e.g., patients with elevated creatinine should avoid high protein diets in certain conditions); patient demographics (e.g., elderly patients should not be recommended highly caloric deficit diets in certain situations); allergies (patients allergic to penicillin should not be given penicillin or amoxicillin); drug-drug interactions (patients on certain drugs should not be given other drugs that might lead to adverse reactions); a form contraindication (e.g., oral, intramuscular); other contraindications. Examples include “do not prescribe penicillin with another particular drug” and “do not administer to children under two years old.”

Accordingly, a therapy restriction node may hold both condition-dependent properties and restrictions on use of its ETIs. In another example, consider the ETIs Excedrin Migraine and Excedrin Extra Strength: they both can be taken to cure migraine but it will be an off-label use of Excedrin Extra Strength while on-label use for Excedrin Migraine. Thus, although both these ETIs will have the same therapy group they will have separate restrictions regarding off-label versus on-label use. Each will have a separate therapy restriction node parent, with the property “FDA Approval” being “yes” for one and “no” for the other.

A therapy restriction node 664 may link to any number of ETIs 665, 666. An elemental therapeutic intervention (ETI) node may include the name of the actual prescribable drug or nondrug therapy intervention, its description, and an ontology link that connects the ETI to the intervention in an ontology (such as RxNorm in the case of drug-based therapies) where a formal description of the intervention is found. In the case of drug interventions, the actual prescribable drug includes at least the actual ingredient of the drug and the dosage. Other aspects of the prescription such as the form, route, frequency, and duration may be included as properties within the ETI, may be in the name or description of the ETI, or preferably are present in the ontology entry. In the case of nondrug interventions, the actual prescribable therapy (which may vary widely depending upon the therapy), e.g., specific name, duration, etc. may also be properties of the ETI, may be its name and description or preferably may be found via the ontology link.

Even though a dosage decision node (e.g., 736 of FIG. 8) has a dosage regimen and a rule link to a rule that calculates the dosage regimen for a given patient after the drug has been selected, an ontology link in an ETI node provides more information. The link connects to a database or ontology with information on adverse effects of a drug and other nondrug properties for nondrug ETIs (which do not have a corresponding dosage decision node). In one embodiment, ETIs are based on branded drug names and not generic drugs. Hence, Tylenol and acetaminophen will use the same ETI for the same or similar dosage regimen. In another embodiment, ETIs may be based upon generic drug names, or may be based upon a mixture of both, for those drugs or therapies not having brand names. Depending upon the condition and types of ETIs, it is possible that a given ETI will link directly to a condition without any intervening therapy group or therapy restriction nodes.

Similarly, an ETI for a nondrug therapy also represents the actual prescribable therapy for the patient. By way of example, ETIs of the type psychological may specify exposure therapy, dialectical behavior therapy (DBT), rational emotive behavioral therapy (REBT) and others. In particular, ETIs may exist for three prescribable types of exposure therapy such as imaginal exposure therapy, in-vivo therapy and virtual reality exposure therapy, each possibly including frequency, duration and other specifics depending upon the type of therapy. A “prescribable” therapy represented in an ETI (whether a drug or nondrug therapy) thus represents a specific recommendation or prescription that a clinician gives to a patient for a particular therapy and its specifics will depend upon the particular therapy.

Even though a therapy group node and a restriction node are shown between an ETI and its linked condition, it is optional to include these two nodes. Properties of a therapy group may be held on an edge or edges between a condition and an ETI. For a condition that does not have groups of therapies that can be grouped conveniently, it may be simpler to link a condition directly to that intervention without having any intermediate therapy group node. If restrictions are not present for a particular therapy than no restriction node is needed. Further, any restrictions may be held on an edge, such as an edge between a therapy group and ETI, or between a condition and an ETI in the case where therapy group and restriction nodes are not present.

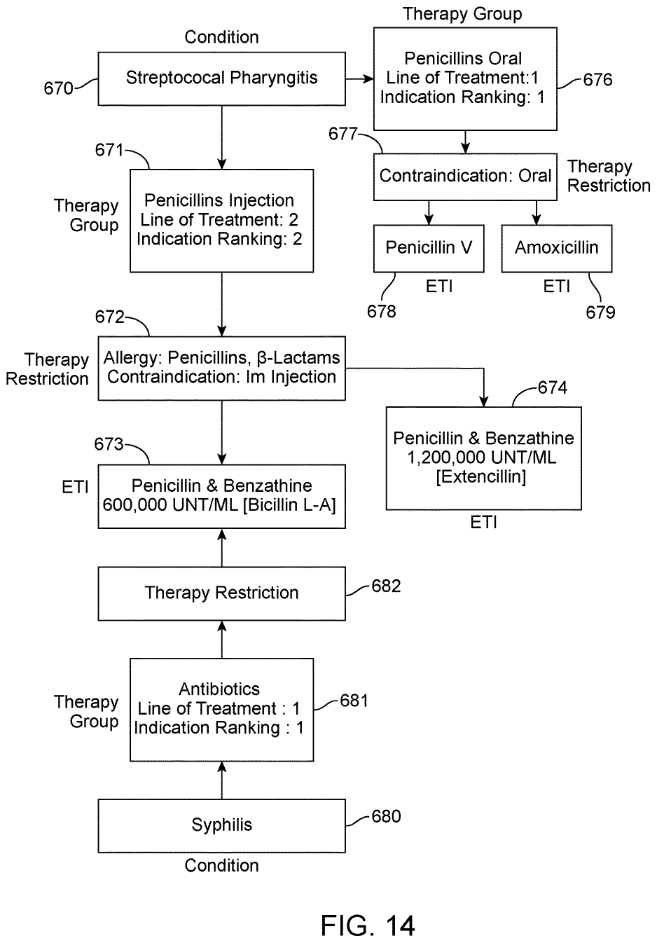

FIG. 14 is another example of a portion of a knowledge graph showing specifics for therapy groups, therapy restrictions and ETIs. As shown, therapy group 671 is for injectable penicillin and thus one of the restrictions in node 672 is a contraindication for intramuscular injections, along with two allergy checks for the drugs below the restriction node. It also includes the condition-dependent properties of line of treatment and indication ranking. ETI 673 is for a particular injectable penicillin ingredient showing the specific units per milliliter and the actual brand name within brackets. The other ETI 674 is for the same drug in a different dosage and having a different brand name within brackets. Note that two different ETIs are used because the dosage is different. Therapy group 676 is for oral penicillin and thus therapy restriction node 677 shows a contraindication for oral delivery; the allergy restrictions for penicillin and β-lactams are not shown. The two ETIs 678 and 679 are for drugs via oral delivery and the specific dosage and brand names are not shown. The therapy group 681 is for antibiotics that treat the condition syphilis 680; the group also has the properties line of treatment and indication ranking specific to the condition syphilis. It has a therapy restriction node 682 having restrictions (not shown) similar to those of node 672. Note that because ETI node 673 is elemental and contains no condition-dependent properties it can be used across conditions, such as conditions 670 and 680.

Other possibilities for a prescription for a condition include: “penicillin V 500 mg oral tablet,” “penicillin V 3 mg/ml ophthalmic solution,” “penicillin V 25 mg/ml oral solution.” In other words, a clinician does not simply prescribe “penicillin V” for a patient; he or she prescribes “penicillin V 500 mg oral tablet,” possibly along with other information such as frequency and duration. And if a drug in tablet form comes in two different dosages, e.g., 250 mg and 500 mg, each will have its own ETI. In general, drugs with different labels are represented by two different ETIs; they may link to the same therapy group though.

Preferably, the actual prescription information such as ingredients, brand name, dosage, form, route, duration, frequency, time of day is held in the entry of the ontology (RxNorm or FDB for drugs) to which the ontology link of an ETI links. Even more information such as class of drug (antibiotic, beta lactam, etc.) is available in the ontology. Similarly, for nondrug therapies, the ontology link contains the detailed prescription information. SNOMED may be used, or SNOMED codes may to some another tertiary ontology or database. This database may be an internal database designed via Web scraping, with the help of clinicians, etc. Therefore, the unique ontology link fetches information about the prescription.

### Computational Guidelines Use Cases

As shown, certain nodes in a computational guideline may link to any ETI, in particular a therapy ingredient option node 774 or a nondrug therapy node such as therapy nondrug node 796 (or any of therapy nodes 780, 784, 790-794). In this fashion, during execution of (or traversal of) a computational guideline, such a particular node may link to and reference an ETI in order to assist with computation, before returning to that particular node to continue with execution of the computational guideline.

The ETIs and other nodes described in FIGS. 12-14 may be used in many different ways, often depending upon the particular use case. One advantage in having a node in a computational guideline link to an ETI node (representing a drug or nondrug therapy) in a clinical knowledge graph is that during execution of (or traversal of) the computational guideline additional clinical data may be captured that is not explicitly present in either the patient state or in the guidelines themselves (e.g., which other conditions are linked to a particular drug or nondrug therapy, which other different restrictions impact a particular drug or nondrug therapy).

Although it is possible during execution of a computational guideline to traverse to an ETI to gather more information from the ETI or to traverse within the clinical knowledge graph from that ETI, the guideline need not explicitly traverse to that ETI but will capture the traversal path within the guideline and store it in patient state service 60 (using unique node identifiers or names) including the link to that ETI; this provides a record of the actual path executed by the guideline in conjunction with the patient state at that time. Better reasoning can then be performed for a future traversal of the computational guideline or in other use cases because there is a link from a node in the stored traversal path to one or more ETIs of the clinical knowledge graph. We can update the patient state with inferences using knowledge available in the clinical knowledge graph via a link to an ETI. Also, a guideline may explicitly traverse to an ETI if necessary in the context of the guideline.

Various use cases are applicable to ETIs including in the context of using stored prior traversals. In a first use case, a link to an ETI in a prior traversal stored in a subgraph is used in a subsequent traversal of guideline for the same or different condition. For example, capturing that someone took macrolide instead of penicillin V for strep in the past indicates that the patient potentially had an allergy to β-lactams in general and this information may be used if that patient visits again for a urinary tract infection (because one line of treatment for UTI is carbapenems which are also β-lactams). A patient presents with a possible UTI and in the context of the patient state from patient state service 60, the query to the knowledge graph may be “has this patient taken a drug for a previous condition that indicates that the patient may be allergic to a certain class of drugs?” The backend engine traverses a link from a macrolide therapy ingredient option in the prior traversal subgraph to an ETI for macrolide. The ETI for macrolide has a parent therapy restriction node providing more details about patients who take or do not take such a drug (e.g., an option for patients allergic to β-lactams, an option for patients with an adherence problem). These details are not present in a guideline; a flow can potentially infer this and perform better reasoning by suggesting a next-best action such as not using penicillin (which is a β-lactam) or using a similar drug with a potential low adherence requirement. The output then is “patient took macrolide for a previous infection and may be allergic to β-lactams” or “patient took macrolide for a previous infection and should consider a drug with a potential low adherence requirement.” Thus, capturing ETI-related information using a prior traversal can guide us with treatments of other conditions by providing more information and inferences.

Or, capturing the fact that the previous traversal used node 434 and that the patient took penicillin V indicates that the patient is not allergic to this drug and that none of the restrictions in node 606 apply (since the patient took the drug). Therefore, the next time the patient presents with a possible condition (which could be the same condition as before), the backend engine knows from the subgraph and by the linkage from node 434 to ETI node 610 that the patient is not allergic to a particular drug, that certain restrictions do not apply, etc., and execution of the computational guideline may occur more quickly and efficiently as certain laboratory tests need not be performed nor certain questions asked.

In another example of this first use case, capturing historic information that a previous traversal is linked to at least one ETI (perhaps via a therapy ingredient option node or another nondrug therapy node) assists a backend engine such as the NBA Engine in reasoning during a future traversal of a computational guideline. The NBA Engine is able to traverse to any condition linked to that ETI, thus providing more than one possibly related condition. By way of example, during execution of a future computational guideline, knowing that the patient traversed to node 434 in the past (via the stored subgraph), the engine may traverse not only to condition 602 but also to any other condition linked to that ETI such as condition 620, thus providing the engine with other possibly relevant conditions and restrictions connected to the drug represented by ETI 610.

This first use case may be implemented by the NBA Engine as follows. In a first step, the NBA Engine performs data ingestion; the engine traverses a computational guideline using the subgraph of a prior traversal by the patient for a previous condition (strep infection) and learns from an ETI that the patient has taken macrolide medication. In a second step for causal analysis the NBA Engine analyzes common medical reasons why patients are prescribed macrolides, then assigns probability values to each potential reason based on the patient's specific condition. (For instance, if the patient is a child, the probability of the child receiving macrolide due to adherence issues is high compared to adults—who most likely receive macrolide mostly due to allergy to penicillin.) A third step is a diagnostic update; using a probabilistic model, the NBA Engine recalculates the overall diagnostic predictions (in this example, the engine determines that an adult receiving macrolide indicates that the patient had a penicillin allergy that will be stored in his or her patient state). A fourth step is a recommendation; based on these updated findings, the NBA Engine recommends the next-best action for the patient's care (in this example, the action avoids giving the patient penicillin or a β-lactam—given that penicillin is a β-lactam).

In a second use case, the NBA Engine performs diagnostics given a patient context, e.g., by retrieving a subgraph of a prior patient traversal of a guideline from the patient state service. Knowing that a patient has been taking the drug HCL for RA for five or six years (by reference to node 564), the engine traverses to the corresponding ETI and its parent nodes in the current knowledge graph, learns other information not in the guideline (such as to which patients not to give HCL or other restrictions on HCL), and then the engine will be able to infer that the person might be at a risk of retinopathy (for example, if the patient should not have been given HCL or has taken it for too long). If the NBA Engine reasons and concludes that an ophthalmology examination has not been done, then it will suggest such an examination as a next-best action. During any review of a prior traversal, the NBA Engine may traverse to an ETI linked to any ingredient or therapy prescribed in the past for a patient, review the ETI and its parents, make any inference based upon information in the ETI (or its parent nodes) compared to patient data, and then recommend a next-best action such as an investigation or request for a factor.

A third use case is an analytics use case. If a user submits a query “find other conditions which can be treated with any ETI taken by a certain cohort of patients,” we first retrieve (or reference) all patient subgraphs stored in patient state service 60 for that cohort of patients whose condition was a bacterial infection (for example). We then match each subgraph to a computational guideline and traverse down that guideline until we find the therapy ingredient option node used by the patient. From that node, we then traverse to the linked ETI (e.g., from node 434 to node 610) taken by each patient and then traverse upward to find all of the conditions that are linked to that ETI. We return all of these conditions as the result. Or, the knowledge graph receives a cohort retrieval query which asks how many patients received a drug with a particular therapy ingredient option and asks for specifics on that ingredient. We retrieve all of the stored subgraphs from the patient state service for the patient cohort and traverse the applicable computational guideline by mapping each subgraph in order to traverse to the particular therapy ingredient option node, if present in the subgraph. We return the number of subgraphs that map to the therapy ingredient option node. We may also traverse from that node to the applicable ETI to gather further information on that therapy ingredient option. Typically, an analytic use case may be any healthcare entity (such as a hospital system) trying to analyze the population of patients stored as subgraphs by asking queries about different nodes in the knowledge graph (conditions, ETIs, EDIs, EDFs of patients). It is close to cohort retrieval except that the output is generally descriptive statistics (count, mean, or any other statistics) rather than fetching actual patients.

In a fourth use case, an engine automatically determines if a patient is being treated for a particular condition given their clinical information. The engine first retrieves any stored subgraphs for this patient from the patient state service representing prior traversals of computational guidelines. By mapping the subgraphs to the relevant computational guidelines, the engine can determine the investigations or observations that were performed (via links to EDIs), the results obtained (via links to EDFs), and the therapies that were administered (via links to ETIs); this information provides hints as to which other conditions also require these investigations, observations or therapies. Patient clinical data from the EHR or patient state service can also be mapped to EDIs and ETIs in the knowledge graph. Knowing the most likely condition can also suggest which treatment guideline to begin traversing.

### Flow Diagram for Determine Diagnosis or Treatment

The below flow diagrams utilize a computer system to execute the computational guidelines embodied within the knowledge graph. The computer system may execute in different manners and may include any number of software modules. In general, the computer system traverses the guideline in order to execute the flows embodied therein, and in one embodiment, the computer system loads the entire comprehensive graph into its memory (for reasons of speed and latency) in order to operate upon it. Or, only a portion of the guideline and knowledge graph (a sub graph) related to the particular condition in question is loaded into memory. In another embodiment, the computer system need not move or load the comprehensive graph into a different location but sends queries and responses back and forth between the computer system and the comprehensive graph in order to execute the computational guideline. Either approach or a combination is feasible and will depend upon the particular implementation. In addition, the computer system will also input patient data from the patient's EHR and this may be done wholesale at the beginning of the process, or the computer system may query the patient's EHR from time to time when particular patient data is needed. In one embodiment, it is NBA Engine 76 which executes the computational guideline.

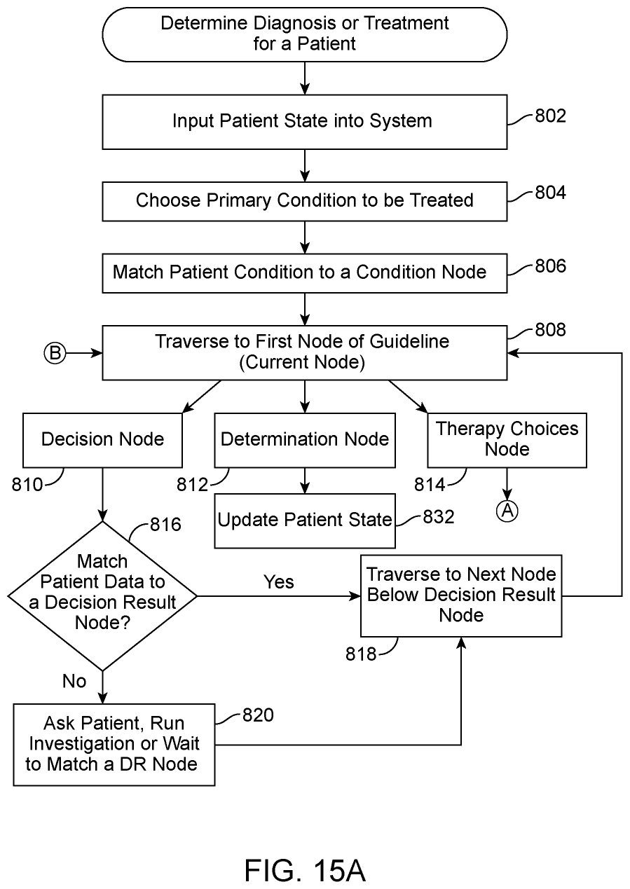

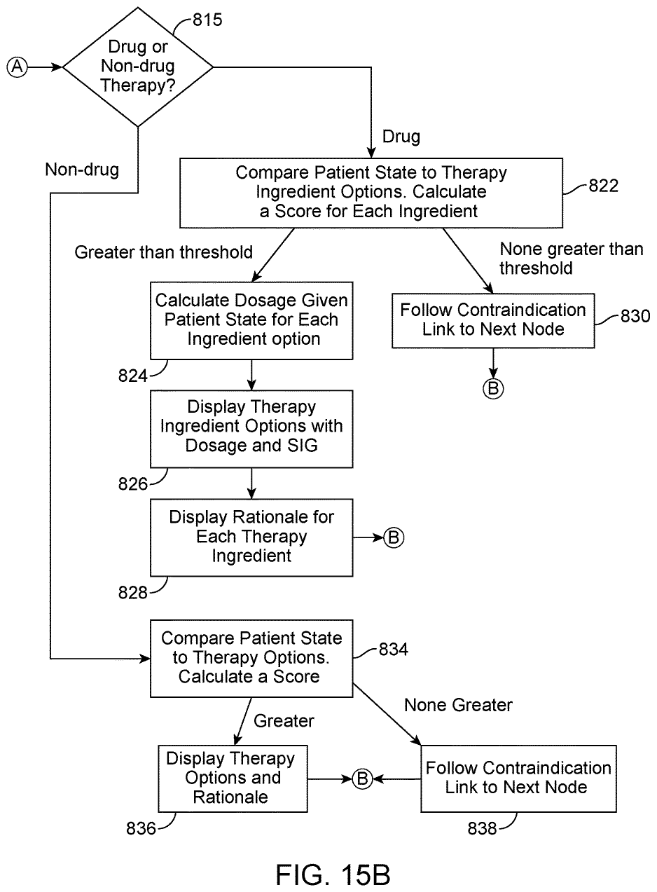

FIGS. 15A-15B are a flow diagram for determining a diagnosis of the patient or a treatment for a particular diagnosis. Accordingly, as mentioned above, the computer system first loads the entire comprehensive graph into its memory or is enabled to send a query to the knowledge graph or guideline and receive a response in order to traverse the guideline according to the flow below. Typically, a condition is suspected and the NBA Engine can select the appropriate guideline to begin with. Otherwise, the engine may wait for that information to be provided either in the EHR or via user interface 24. The below will reference the guidelines in FIGS. 5A, 5B, 6A and 6B.

In a first step 802, a computer system inputs the current patient state. The current patient state will typically be input from the patient's EHR and will include standard patient state information such as known present or absent diagnostic and risk factors, results from laboratory tests or examinations, family history, patient history, narrative clinical documents such as patient's chart, diagnostic reports, discharge notes, etc. Such an input from the EHR may be performed as described herein and use the NLP and OCR modules shown in FIG. 1, for example.

The patient may present for a medical visit with a clinician, e.g., an in-person visit or by telephone, computer, or any other suitable telemedicine scenario. Alternatively, the patient has presented in the past and the clinician is reviewing the patient's chart without the patient being present. Or, the flow is being executed upon the patient's EHR without any patient available and any needed answers are supplied automatically, using default answers, or by a best guess by a clinician.

The clinician may use EHR system user interface 20 (or other suitable user interface such as application 24) to open the patient's electronic chart in the EHR system. The EHR system detects the clinician's context, the patient context (e.g., a medical record number) and sends this information to the backend systems of platform 10. Platform 10 downloads the patient's medical history from his or her chart and transmits the textual information to the NLP engine 74 via patient state service 60. Included within this medical history are any clinical notes that a clinician had entered previously or has just entered, including simple notes, SOAP notes, structured column notes, consultant notes, etc. Further, any transcriptions may be included as well as transcriptions transcribed from a microphone using speech recognition software. The platform extracts the diagnostic factors, risk factors and investigation results (including laboratory test results and examination results) from the patient's medical history using inferential reasoning, resulting in factors and investigations that are either known to be present or known to be absent. The clinician may also decide to enter patient data into the EHR via clinical co-pilot 24 from which the patient data is fetched using EHR APIs.

In a next step 804 a primary patient condition to be treated is chosen. Typically, any known or suspected patient conditions will be included with the current patient state if they are part of the patient's EHR. Or, suspected conditions may be determined as described herein or as described in U.S. Pat. No. 11,869,674 (attorney docket No. RCVRP002) in which case the most likely condition may be chosen. Or, the patient may suggest a particular condition during an in person or online visit, the clinician may suspect a particular condition during a visit, and that suspected condition or conditions may be entered into the computer system for processing.

The clinician may choose a likely condition and input it into the computer system; by default the computer system may choose the most likely condition from the patient state or may choose the only condition from the patient state. The chosen condition may be a suspected condition that the guideline will attempt to confirm or rule out, or the chosen condition may be known in which case a guideline will implement a treatment for that condition. If the entire knowledge graph has not been loaded into memory or moved by the computer system earlier and it is desired to do so, at this point a subgraph of the knowledge graph pertaining to the chosen patient condition may be loaded at this time. It is the responsibility of the NBA Engine to determine a suspected or known condition by scanning through the patient state. Pattern recognition models may be used to do this.

In a next step 806 the chosen patient condition is matched to a node in the knowledge graph, e.g., the known condition of acute pharyngitis is matched to node 402 (or RA is matched to node 502). Next, in step 808 we pick a guideline and traverse to the first node of that guideline, e.g., the clinician selects the “prescribe a drug” guideline and we traverse to node 408. Or, the first node is node 504 where a decision needs to be made. Node 404 may be selected by the clinician or may be the default with a known particular condition. In another example, the first node is actually decision node 504 in order to select particular guideline. The first node of the guideline (or the next node as the flow loops and traverses down the guideline) may be a decision node 810, a determination node 812 or a therapy choices node 814.

If a decision node, then in step 816 the NBA Engine attempts to match any known patient data to one of the decision result nodes of that decision node. By way of example, if at decision node 408, the system attempts to match one of the decision results (severe allergy, no allergy or mild allergy) with what is known in the patient's EHR. In one embodiment, each decision result node contains an ontology link to the particular concept (e.g., “no allergy to penicillin”) which provides a code, this code may then be matched to the corresponding code in the patient's EHR, if in fact the patient has no allergy to penicillin. If there is a match in step 816, then in step 818 the system traverses to the next node below that decision result node, e.g., to node 420. (Or from node 514 to node 520 and on down to node 522.) Control then moves back up to step 808 where this next node becomes the current node and the flow continues depending upon whether that current node is a decision node, a determination node or a therapy choices node, as above.

On the other hand, as if there is no match in step 816 because there is no or insufficient patient data in the EHR, then in step 820 that patient data may be obtained in a variety of manners depending upon the type of decision result needed from the patient. By way of example, if at node 408 it is unknown whether not the patient has a penicillin allergy, then the clinician may ask the patient and enter the answer directly into the patient EHR, directly into the system in response to a query from the system, or in some similar manner. If the decision to be made is based upon a laboratory test or examination (such as decision node 420), and that investigation has not been performed yet, then the system may recommend, direct, automatically schedule or automatically perform that particular investigation in order to obtain a result for that patient. By way of example, the system would direct the clinician to order a penicillin susceptibility laboratory test and when that result is returned to the patient EHR then a match can be performed with any of nodes 422-426 and processing may continue. Once the match occurs, then in step 818 the system traverses to the next node below the decision result node, control returns to step 808 and processing continues as above. In the case of a decision node which is a decision delay (e.g., node 566), the system pauses processing, outputs a note to a clinician, automatically adds a future date to calendar software, periodically checks the patient EHR, or in any similar fashion delays until the particular time period in a decision result node has passed and then begins processing again. By way of example, at node 566 processing is paused until three months have passed since the patient was prescribed the medication.

In this fashion, execution of the flow embodied in the guideline continues down through the nodes matching decision results to patient data in the EHR when available and then prompting for patient diagnostic or risk factors or requesting that investigations be run when that data is not available in the patient EHR. In a live environment, the clinician is able to question the patient or order investigations which can be completed fairly quickly, in which case processing may continue. Alternatively, if there is no match then processing may end and the system may output a message indicating which information is needed and the processing has ceased. The clinician may begin processing once the information has been obtained. Or, in a prediction application, processing may continue when the system predicts the probability of the next node and then chooses the path with the highest probability. Or, in a population analysis application or in an historical information analysis, it may not be possible to query a clinician for patient information, in which case the system may simply make its best guess of a probable result using probability, use a default or generic answer, or use some other estimating technique.

Returning now to node 808, it is possible that the first node or the current node is a therapy choices node 814 (e.g., node 428 or node 562) and control moves to 815. If a drug therapy choices, then control moves to step 822. In order to choose a particular therapy ingredient for the patient for this condition, we compare the patient state to each of the therapy ingredient options below the therapy choices node, e.g., compare the patient state to penicillin V, penicillin G and amoxicillin in order to determine which is the best drug for that patient for this condition. A score is calculated for each ingredient and may be based upon: how allergic the patient is to that drug (a contraindication), how effective that drug may be against the condition, and other information such as if a patient is already taking another drug then a drug-drug interaction can deter usage of certain drugs. Other reasons that guide the drug selection process can be provider preference, patient preference, dosage type preference (oral vs. intravenous), etc. Each score for each ingredient is compared to a threshold and for those ingredients with a score greater than the threshold (i.e., the risk is low enough), control moves to step 824. Thus, the score is not definitive, but more of a recommendation to the clinician.

Next, in step 824 a dosage and SIG (the prescription) is calculated for each ingredient option based upon the patient state. The dosage may be calculated in different manners. In one particular embodiment, for each therapy ingredient option having a score greater than the threshold, we send the ingredient, the condition that we are treating, and the relevant patient state information to an external service which will then recommend a dosage. This service may also supply the SIG as well. An example dosage is: medication name—penicillin V; route—oral; dose amount—500 mg; frequency—twice a day; and duration—10 days. Typically, dosage is a function of the patient's age, gender, weight, past medical history, allergy profile, etc. For example, a child may be given half the dose amount as compared to an adult for a given medication.

In step 826 the system outputs or displays to the clinician each of the therapy ingredient options with a score greater than the threshold along with its score and the prescription for each. This output may be modified to include any or all of the ingredient, the score, the dosage or the prescription. In addition, other information for each drug ingredient may be included such as drug availability, side effects, cost, insurance coverage, etc.

In step 828 a rationale for each therapy ingredient may also be output or displayed to the clinician. For instance, the output may indicate that a particular ingredient reacts more strongly with the condition than another ingredient and is thus more effective and may use the description information in the therapy ingredient nodes. Now that the therapy ingredient options, dosages and rationale have been displayed to the clinician, the drug is prescribed and the patient takes the drug. The next node may be a determination node or a decision node (such as a decision delay) and thus control returns to step 808 for a determination of what is the current node and processing continues as above.

Returning to step 822, if none of the scores for the therapy ingredient options are greater than the threshold then control moves to step 830 and the contraindication link or links are followed to the next node to allow a different drug to be tried. By way of example, if none of the nodes 430-434 score above the threshold, then the contraindication links are followed to node 438 for the alternative drug cephalosporin. This node then becomes the current node and control returns to step 808.

If at step 815 the node is a nondrug therapy choices node, then control moves to step 834. As in 822, we compare the patient state to each of the therapy options below the therapy choices node (e.g., compare the patient state to node 634) to determine which is the best therapy for the patient. A score is calculated for each therapy and may be based upon: any contraindication, how effective is the therapy, ability of the patient to tolerate the therapy, other therapies already in use, etc. Other reasons may be provider preference or patient preference. Each score is compared to a threshold and for those therapies with a score greater than the threshold control moves to step 836.

In step 836 the system outputs or displays to the clinician each of the therapy options with a score greater than the threshold along with its score and the prescription for each. This output may be modified to include any or all of the therapy, the score, or the prescription. Other information for each therapy may be included such as recovery, side effects, cost, insurance coverage, etc. Also output is a rationale for each therapy. Now that the therapy options and rationale have been displayed to the clinician, the therapy is prescribed and the patient undergoes the therapy. The next node may be a determination node or a decision node (such as a decision delay) and thus control returns to step 808.

Returning to step 834, if none of the scores for the therapy options are greater than the threshold then control moves to step 838 and the contraindication link or links are followed to the next node to allow a different therapy to be tried. This node then becomes the current node and control returns to step 808.

Returning now to step 808, if the first node or the current node is a determination node 812 then control moves to step 832 where the patient state is updated based upon the determination (e.g., RA confirmed or RA absent); the flow then ends. Patient state is updated via direct updates in the EHR or by providing information via the chat feature of co-pilot 24 by the clinician. The patient state is also stored in patient state service 60 along with a record of the subgraph traversed by this flow as described above.

### Predict Patient Trajectory and Compare Patient Trajectories

A future patient state (or patient trajectory) may be predicted through the use of transition probabilities used within a guideline. Referring again to FIG. 5A, each link (or edge) which is part of a guideline has been annotated with a particular percentage.

By way of example, the edges from decision node 408 have been annotated with the percentages 5%, 75% and 20%. Each percentage indicates the probability, likelihood, or expectation that a person having a state of a particular node will transition to one of the linked nodes. In other words, there is a 75% chance that a person being tested for a penicillin allergy will have no allergy to that drug. Or, considering a penicillin susceptibility test at node 420 in the context of the particular condition, there is a likelihood of 40% that the sample tested will be susceptible to penicillin. Or, considering the penicillin therapy choices node 428, there is a 40% chance that the drug penicillin V will be chosen. Or, for a person being prescribed amoxicillin at node 432, there is a 90% chance that he or she will improve and a 10% chance that he or she will not improve (a contraindication) and that the sample will be tested for susceptibility to the drug cephalosporin Gen1 at node 438. Nodes that have only a single link to another node have not been labeled as it is assumed that the probability is 100%. Links to EDFs and EDIs are not annotated with a percentage. A contraindication may mean that the patient has an allergy to a drug (e.g. penicillin), that a drug does not work 10% of the time and you have to move to a different drug, that there is an adverse reaction with another drug or that there is a drug-food interaction.

Depending upon the implementation, generic probabilities may be used for all patients, although each transition probability may be modified as per patient demographics. By way of example, if 75% of the general population has no allergy to penicillin but children under the age of five have an 85% probability of not being allergic to penicillin, then the transition probability from node 408 to node 414 may be modified for any patient who is a child under the age of five to be 85%. Demographics such as age, gender, weight, height, etc., may be used to modify these generic probabilities, and the modification for each link between nodes may be different. In other words, because a child is less allergic to penicillin does not mean that he is less allergic to another drug. These generic probabilities may be attached to the edges between nodes and probabilities modified as per patient demographics in different manners. The generic probabilities may be entirely empirical, derived from data gathered from third-party clients or even customers, or extracted from existing literature. Further, it is possible to have some probabilities come from data while the rest come from literature.

These transition probabilities may be annotated to, attached to, or stored within the knowledge graph in a variety of manners. In one embodiment, each transition probability is a property of an edge between two nodes, e.g., the probability 40% is a property of the edge between nodes 420 and 424. Or, that probability is a property of node 420 but is identified with the edge leading to node 424. Or, that probability is a property of node 424 but is identified as being the probability of originating at node 420. Other techniques are possible, as long as when one traverses from node 420 to node 424 (for example) the probability of 40% is collected and represents the probability of transitioning from node 420 to node 424.

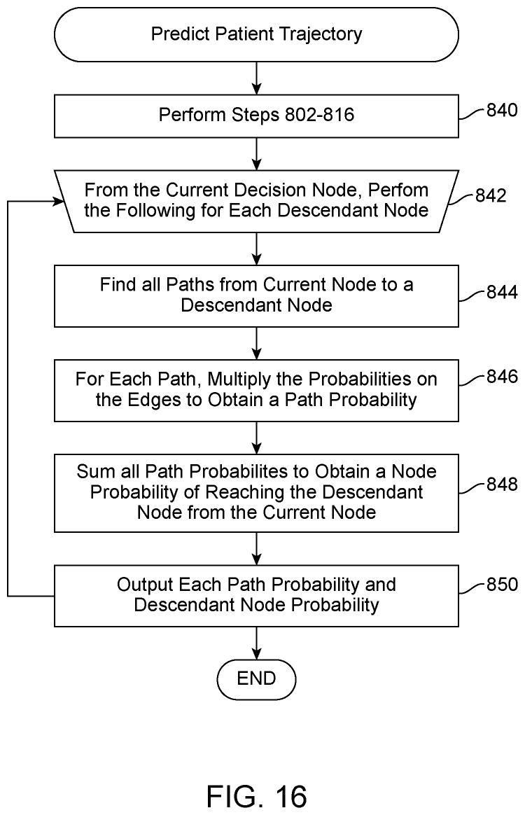

FIG. 16 is a flow diagram describing how to predict a patient trajectory. This flow will predict a patient future state in the context of a particular guideline, which may be for diagnosis, for treatment or a combination of both, of which many are illustrated and described herein. Such a prediction may be used by many different entities: a clinician may use this prediction to understand what is the likely outcome for a patient who is currently being diagnosed or is undergoing treatment (e.g. waiting for test results to come back, predicting what a diagnosis might be, predicting which treatment may be most likely, etc.); a hospital, clinic or insurance company predicting what will be the likely diagnosis or treatment for a patient (for planning, cost estimating, etc.); an insurance company predicting the cost of a patient cohort to understand their future underwriting costs (e.g., for patients diagnosed with rheumatoid arthritis at node 538, what is the most likely drug they will be using and what is its cost).

This prediction may also be used in other scenarios: medical device manufacturers and pharmaceutical companies may use the prediction to understand market potential for their products in specific patient cohorts; researchers may use the prediction to identify patient cohorts for specific studies or to understand disease progression in real-world settings; quality improvement teams may use the prediction to identify areas for potential intervention or process improvement in patient care pathways; patients themselves may use the prediction (when appropriately presented) to understand their likely disease course and make informed decisions about their care options. A request for a patient trajectory may originate automatically from a claims (or other) database or via user interface 80b, as described in FIG. 16.

A prediction of a patient state may begin at any particular node within a computational guideline, although starting farther down the guideline after matching all available patient data from the patient EHR will yield the most accurate prediction. By way of example, beginning at node 502 or perhaps node 514 when a patient is suspected of having rheumatoid arthritis (and before much diagnosis or treatment) will yield a great deal of predicted paths and probable descendent nodes, but may not be very useful in predicting the path of a particular patient. On the other hand, if data from the patient EHR indicates that the patient has progressed to node 588 and has retinopathy after using HCL, then prediction from that point will yield fewer paths and fewer descendent nodes, thus providing better information for the clinician or other entity. Thus, in a first step 840, steps 802-816 from FIG. 15A are preferably performed, traversing the guideline and matching all available patient data until in step 816 there is no available patient data to match to a decision result node. In other words, at some point the flow will arrive at a decision node and there is no patient data in the EHR to provide a needed diagnostic factor, risk factor or investigation result (e.g., the clinician needs to ask the patient a question, examine the patient or run a laboratory test); since this is the current state of the patient, beginning the prediction from this point yields the best results for the clinician.

As mentioned above though, in step 840, the clinician or other entity may direct that a node higher up in the knowledge graph should be the current decision node, or the clinician may assume that certain diagnostic factors will be present or absent, that a laboratory test will yield certain results or that a certain treatment will not work, etc.), and may provide input that the prediction should start farther down the knowledge graph. By way of example, should a patient begin treatment for rheumatoid arthritis with HCL but the clinician suspects that it will not be effective, may be too expensive, may have certain undesirable side effects, etc., the clinician may direct that the prediction begin with a current node of 560 in order to understand if the drug methotrexate might have a better probability of success. The clinician may direct that prediction should begin with a different node; for instance, the clinician has an option to do so via clinical co-pilot 24. He or she can also select a different treatment guideline or a different node in the same or alternate guideline to indicate his or her preference.

Assuming though, that steps 802-816 are performed, the flow arrives at a current decision node (such as node 420, using the example of FIG. 5A) for which there is no match in the patient EHR (in this example, the penicillin susceptibility test has not been run yet). Next, in step 842, the following steps are performed for each descendent node. Generally, a descendent node will be each node below the current decision node, such as nodes 422-449 in this example. Or, an implementation of this prediction may define a descendent node as only being a leaf node, such as nodes 436, 440, 448 and 449 in this simple example. Defining descendent nodes as only being leaf nodes yields fewer trajectory paths and fewer descendent node probabilities, thus not overwhelming the clinician with too much information. Or, descendent nodes may be defined as only being determination nodes, thus yielding only probabilities of reaching a confirmed condition or an absent condition, for example. Or, descendent nodes may be defined as only being therapy choices nodes (e.g., nodes 428 and 446), thus yielding only probabilities of prescribing certain drugs. In general, descendent nodes may be defined according to the particular implementation.

In step 844, we find all paths from the current node to each descendent node (e.g., each node 422-449). To use a simplistic example, all paths from the current node 420 to a descendent node 436 (“patient improved”) include a first path through nodes 426, 428, and 434, a second path through nodes 426, 428 and 432, a third path through nodes 426, 428 and 430, and three more paths beginning at node 424 in a similar fashion. Each of these paths is a possible trajectory of the patient in the future to arrive at node 436. Next, in step 846, for each path, we multiply the probabilities of all the edges to obtain a path probability. For example, for the path through nodes 426, 428 and 434, we multiply 40% by 100% by 40% by 90% to obtain a path probability of 14.4%, this means that there is a 14.4% chance that a patient with acute pharyngitis waiting for a penicillin susceptibility lab test to be performed will see improvement by taking penicillin V if he or she follows that particular path. For the path through nodes 426, 428 and 432, the path probability is 10.8%. Transition probabilities for all of the other paths to node 436 are also calculated in the step.

Next, in step 848 all of these path probabilities calculated in step 846 are summed in order to obtain a node probability of reaching particular descendent node 436 from the current node 420. If the two paths above were the only two paths, then the two probabilities are added together to obtain a node probability of 25.2%. This indicates that if only two paths are available, that there is a 25.2% chance that a patient at node 420 will see improvement by taking one of the two drugs. As more paths are available in this simple example, all those path probabilities from node 420 to node 436 are added together to obtain the actual node probability.

In step 850, all of the path probabilities and each descendent node probability is output. Output may occur in many different manners. These probabilities may be output on a display of a computing device for viewing by a clinician, patient, administrator, etc., the relevant portion of the guideline may also be displayed showing each particular path being highlighted and annotated with its particular path probability, and each descendent node being annotated with its node probability, or in a similar fashion. Only the node probabilities may be output along with the name of each node, or only the path probabilities may be output along with the name of the final descendent node and optionally names of any nodes along each path. Output may also be electronically from the system to another computer system, database, LLM, API, or similar interface to a corporate entity desirous of this information. And, the output path probabilities or node probabilities may also be sorted (e.g., highest probabilities first), and the node probabilities may be filtered to only display node probabilities of determination nodes, therapy choices nodes, etc. (in the case where all descendent node probabilities have been calculated).

Delay decision nodes may be handled in different manners depending upon the preferred implementation. Node 566 has two edges and each will be annotated with a transition probability indicating the likelihood of transitioning after a three-month delay or after a five-year delay, respectively. In one embodiment, each edge may be labeled with 100% as it is nearly certain that the particular time period will have passed and the trajectory may continue down that path. Or, if it seems unlikely that a trajectory will continue down a path with a multiple year delay (e.g., node 570 and the patient is very elderly), then the edge to node 568 may be annotated with 90% and the edge to node 570 annotated with 10%, or similar. Or, as time marches on, and the five-year delay is imminent, then the edge to node 570 may be adjusted and its probability changed to 90% or higher. Or, if a particular time period has already passed (e.g., six years have passed), then the edge to node 570 may be annotated with a 100% probability. In the case of edges from decision delay nodes, the sum total of all edges need not add up to 100% and annotation of their edges may depend upon the particular implementation.

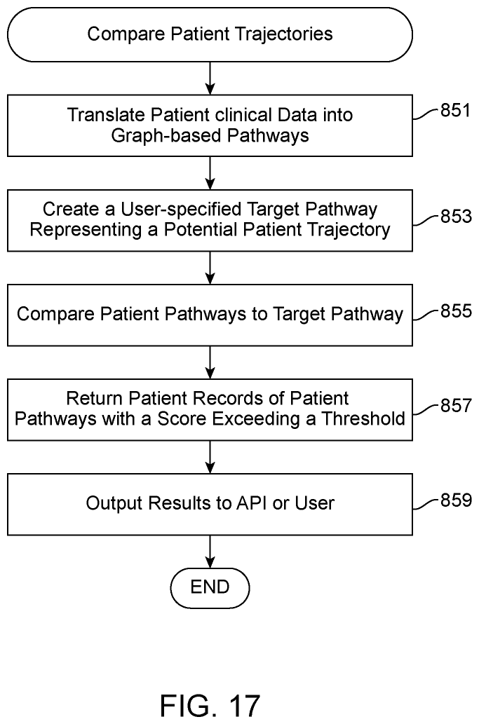

FIG. 17 is a flow diagram describing how to compare prior patient trajectories with a user-specified or “target” trajectory. The goal is to determine how many, or which, patients have followed the target trajectory in the past. By way of example, the pool of patients are those in a particular trial (trying a new drug, for example), are those in a particular hospital over the past year, are those having a particular medical condition, and it is desired to determine those that have followed a particular trajectory in a particular diagnosis or treatment guideline (typically, the guideline relates to the pool of patients, i.e., a treatment using the new drug, a treatment for a particular medical condition, a diagnosis based upon a suspected condition, etc.) Trajectory Engine 71 may also include appropriate software and reasoning to perform this flow diagram.

In a first step 851, patient clinical data for any number of patients is translated into a graph-based pathway per patient. A query input from a user interface first specifies the particular pool of patients in question (by patient identifiers, a date range of admission, a control identifier, a condition in common, etc.), along with the particular computational guideline to be used to generate the pathway, and a request to compare patient trajectories. A patient's clinical data may be that data found in the patient EHR, in a clinician's notes, or in other locations mentioned herein. For each patient, the clinical data is translated into a graph-based pathway, i.e., the nodes and edges of the particular computational guideline, specifically a subgraph described above that represents the historical trajectory of that patient using the nodes and edges of the computational guideline. In one embodiment, the patient's raw clinical data is first translated into a graph-based representation of nodes and edges as shown herein, and that representation is then converted into a subgraph. Or the raw clinical data is directly translated into the subgraph representation. For example, medical codes in the patient clinical data are mapped to nodes in the computational guideline in order to create a pathway. NLP may also be used to translate raw clinical data (e.g., a clinician's notes) into more structured data better for matching against nodes of the guideline. In this case, NLP Engine 74 may be used by Trajectory Engine 71; the NLP Engine may be trained in the domain of medical concepts and preferably is trained using nodes of the computational guideline in order to better identify similar concepts within the raw clinical data.

In a next step 853, a clinician, administrator, researcher or other user creates a user-specified target pathway within the computational guideline specified. This target pathway within the guideline thus represents a pathway of interest to be compared with all patient pathways. Given the particular computational guideline, the user selects the target pathway node-by-node and edge-by-edge within the guideline, and the resulting target pathway is stored as a target subgraph. Preferably, this target pathway uses the same subgraph representation as the patient pathways. For example, given a graphical representation of a particular computational guideline (such as any of those shown herein), a user selects a pathway node-by-node within the guideline by clicking upon, selecting, highlighting, or otherwise indicating each node, then indicating that the selection is done. The user may also select a target pathway by retrieving a stored subgraph, submitting a list of nodes identified by their identifiers, and in other manners. Next, in step 855 each of the patient pathways is compared to the target pathway using their respective subgraphs for comparison. Any of a variety of comparison algorithms may be used, including similarity algorithms, and other algorithms able to compare subgraphs that represent graphical representations, able to compare lists of node identifiers, etc.

In step 857 the engine returns those patient records having a patient pathway matching the target pathway with a similarity score exceeding a particular predetermined threshold. The engine may return simply a patient identifier, name or similar, may return a link or other identifier of the patient EHR, or the entire EHR or a portion of it. In step 859 the engine outputs this results to an API, to the computer or user interface used to input the original query, or to the user in any suitable fashion.

### Example Use Cases

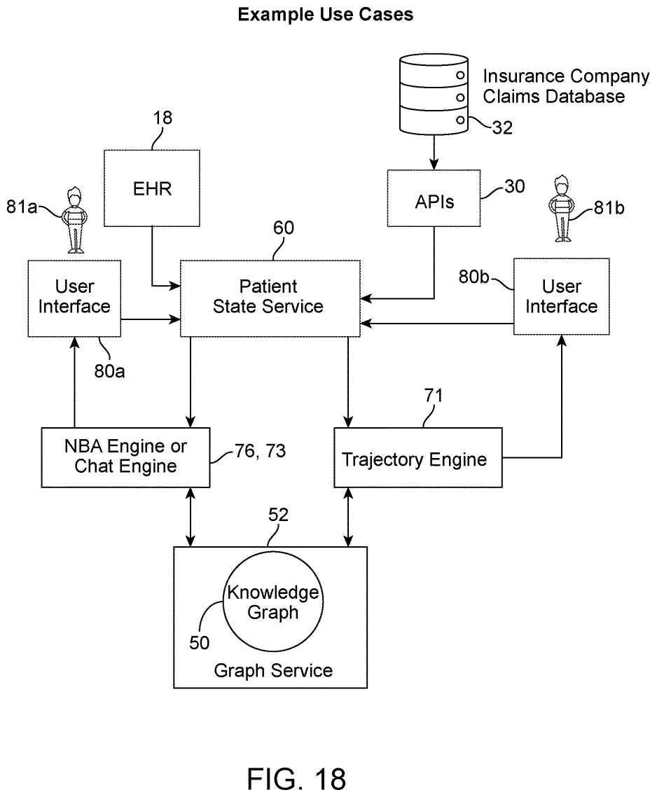

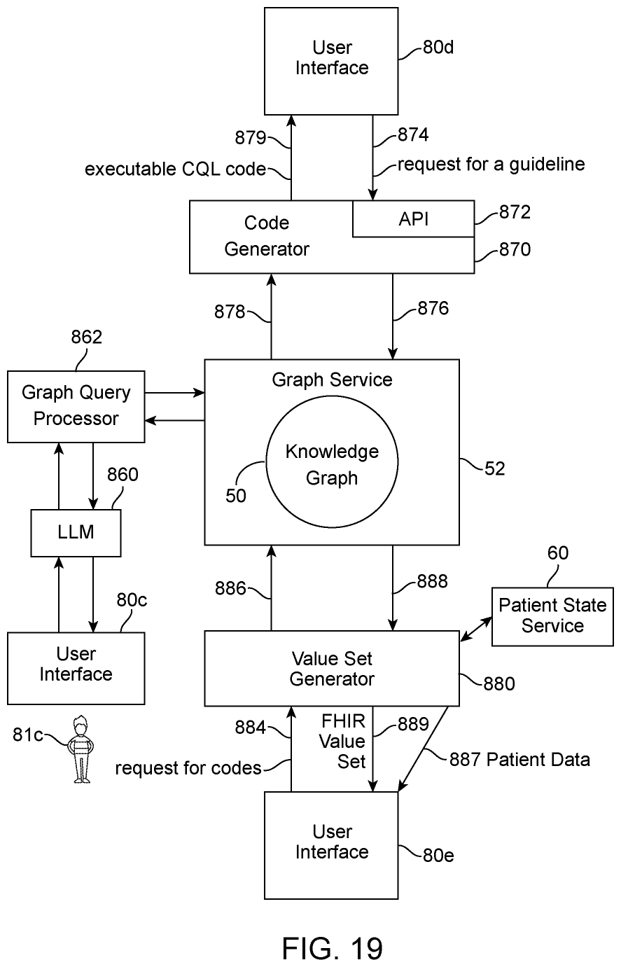

The comprehensive graph described herein supports a wide variety of use cases, whether a query is initiated by a human or by computer, or whether the output response is directed back to the human, to a software application, to a computer, to a database or to some other electronic device in electronic form. In general, a medical query is input, an application executes upon that medical query using the comprehensive graph (and optionally patient data) and results are output for display to a user, output to an API, to an LLM, to another computer, etc. Generally, use cases include a first category shown in FIG. 18 in which a user queries the knowledge graph in the context of a patient or patients and makes use of a backend engine such as NBA Engine 76, Trajectory Engine 71 or Chat Engine 73 in order to perform reasoning, and a second category shown in FIG. 19 in which a user queries the knowledge graph itself without using any patient context or reasoning about the patient.

FIG. 18 is a block diagram showing three example use cases involving the patient state. As explained above, the computational platform includes the comprehensive graph (i.e., the clinical knowledge graph linked with any number of computational guidelines) as well as graph service 52 providing an interface between the comprehensive graph and other software applications. In a first use case, a clinician 81a uses a computer with a user interface 80a (e.g., clinical copilot 24) in order to submit a medical query to the platform via patient state service 60. The query is fielded by the NBA Engine 76 which performs the reasoning based upon the input medical query using the knowledge graph and the result is then output. By way of example, NBA Engine 76 is arranged to implement the flows described in U.S. Pat. No. 11,869,674 (attorney docket No. RCVRP002) such as determine patient state, determine patient conditions (including differential diagnoses), determine next-best actions, etc. NBA Engine 76 is also arranged to execute any of the computational guidelines described herein, including FIGS. 5A, 5B, 6A and 6B, and to implement any of the flows described herein including those in FIGS. 15A and 15B (e.g., determine a diagnosis, a treatment, etc.) and the flows regarding the ETIs of FIGS. 12-14. Once NBA Engine 76 has a result it outputs that information back to a computer, and in this example, to a user interface. In addition, the clinician may submit a query via the user interface 80a via Chat Engine 73 arranged to handle straightforward conversations with contextualized patient data from the patient state service 60. The Chat Engine may be implemented using an LLM in which case it can handle natural language queries from the clinician; a graph query processor 862 may be used in between the Chat Engine and the comprehensive graph.

In a second use case, similar to the first use case, a clinician 81a uses a computer with a user interface 80a (e.g., clinical copilot 24 or other similar software) in order to submit a medical query to the platform via patient state service 60. The query is handled by a software engine such as a cohort retrieval engine which performs the reasoning based upon the input medical query using the comprehensive graph and the result is then output. By way of example, any medical query input (such as those described above regarding ETIs) may be input. Once the cohort retrieval engine has a result it outputs that information back to a computer, and in this example, to a user interface 80a. For instance, a user 81a submits a cohort retrieval query which asks “how many patients received a drug with a particular therapy ingredient option?” For a simple query of this type, the engine queries patient state service 60 for each patient subgraph traversal previously stored, and for each subgraph attempts to match the node unique identifier associated with that particular therapy ingredient option with a node in the subgraph; a match indicates that the patient associated with that subgraph did receive that drug. For a more complex query (e.g., “find all patients suffering from a particular condition for more than two years taking a particular drug”), an analysis of each subgraph involve more steps such as matching the particular condition to the subgraph, finding the particular therapy node matching the drug, and performing a calculation on how much time has passed since the patient was first diagnosed with condition. In general, the engine performs matching to answer the query by mapping each subgraph to the computational guideline and determining if each subgraph traverses nodes in the guideline that correspond to elements of the query and performing any calculations if needed. A medical query may also be input that does not rely upon our use any patient state.

In a third use case, an insurance company or other entity queries the comprehensive graph to predict a patient trajectory or trajectories or to compare patient trajectories; the query typically originating from claims information in a claims database 32, being submitted automatically via APIs 30 to patient state service 60 via a Trajectory Engine 71, which is a software module arranged to operate upon the knowledge graph and execute the flow of FIG. 15A, for example. A user 81b may also submit the claims information using a patient trajectory user interface 80b. Claims database 32 typically includes details on patient visits, procedures, prescriptions and costs for the insurance company and patient. As mentioned above, trajectory engine 71 can choose to order or filter any of the output information for display to the user via interface 80b.

FIG. 19 is a block diagram showing other example use cases that do not necessarily use a patient state. In a fourth use case a clinician 81c uses a user interface 80c of a computer to ask questions of a large language model 860, which in turn outputs the query into graph query processor 862 (an API) which queries the comprehensive graph via graph service 52. Results return via the processor 862, back to the LLM, which outputs results in plain English to the user. This LLM use case is a standalone content-querying use case that does not require any interaction or reasoning with patient context; a clinician, researcher, or medical student can query for answers or content for medical conditions and treatments without needing access to specific patient data. Hence, there is no reasoning engine needed in between the LLM and the knowledge graph as in other use cases. Graph query processor 862 takes in a graph query and executes it against the clinical knowledge graph. Some applications may require communicating in natural language in which case LLM 860 or similar software will be used to translate the natural question to a graph query before its answer is fetched from the graph using the processor. It is used primarily when we need to fetch information from the graph. Chat Engine 73 may be implemented using an LLM in which case Chat Engine 73 may take the place of LLM 860.

The output from the comprehensive graph to the graph query processor may be in the form of a data structure, such as a dictionary of attributes on a node, a list of nodes that are each a dictionary, etc. This may include any information from the clinical knowledge graph as well as from any of the computational guidelines described herein (such as the streptococcal pharyngitis guideline of FIGS. 5A and 5B, the rheumatoid arthritis guideline of FIGS. 6A and 6B), and from any other guideline for any medical condition implemented as described. This output is then passed from the processor to the LLM. The LLM references this data structure when formulating its response to the user's original question. An example query from a user and response from LLM 860 are as follows. User: “Fetch me all drugs prescribable to patients suffering with strep pharyngitis who are severely allergic to penicillin.” System response: “For such a severe allergy consider Macrolides such as Clindamycin, Azithromycin or Clarithromycin.”

In a fifth use case, the platform 10 generates executable CQL (clinical quality language) code 879 based upon a request for a particular guideline 874 from the comprehensive graph. A hospital, clinic, clinician or other entity wishes to obtain an executable form of the diagnostic or treatment guideline represented within the comprehensive graph that may be run upon Fast Healthcare Interoperability Resources (FHIR) which is typically the standard way that patient data is stored in an EHR. The entity may wish to execute this CQL code on its own computer system over and over again in order to understand how to diagnose a patient and what treatments should be prescribed, in order to analyze a cohort of patients, etc.

Query 874 is typically a natural language query from a human using a computer, may use keywords, and may also originate from a computer automatically without human input. By way of example, a user inputs a query 874 requesting CQL code to execute a guideline for a particular condition, e.g., a request for the “prescribe a drug” guideline 404 for the condition 402 as shown in FIG. 5A, or a request for the AHA guideline 506 for the condition rheumatoid arthritis 502, or a request for the treatment guideline for rheumatoid arthritis beginning at node 538. The condition may be identified by name, by its unique identifier, or in a similar fashion, and the guideline may be identified by name, unique identifier, etc. The output will be executable CQL code 879 that represents the requested diagnostic or treatment (or combined) guideline represented in a portion of the knowledge graph which has been generated (or “transpiled”) into the CQL code.

Upon receipt of this request 874 via its API 872, code generator 870 submits this request 876 via graph service 52 to the comprehensive graph. The response 878 is a grouping of all nodes, edges and values of that portion of the knowledge graph corresponding to the requested guideline, that is, all of the information contained in that portion of the comprehensive graph, e.g., nodes 402-449, nodes 502-590, or nodes 538-590. The information may be returned in a variety of manners: nodes and edges, with all properties and values; lists of triples, each triple including two nodes and the edge between them; a list of all nodes along with a list of all edges; etc. As this is a representation of the comprehensive graph, it does not include patient specific data. Response 878 may include the links and nodes corresponding to the EDFs, EDIs, ETIs that are referenced by this guideline.

Upon receipt of this representation of a particular guideline for a condition, code generator 870 converts this representation into CQL code able to execute the requested guideline. To begin, the generator may input the representation and convert it back into graph format or may operate upon the lists of nodes and edges directly. The generator steps through the representation of the guideline, from node to node, and based upon a set of rules, creates lines of CQL code. By way of example, considering the decision node 408, a standard function template converts that node along with its three results into the corresponding CQL code. Or, starting with the three edges, three different functions are used to convert node 408 and its three results into CQL code. Appendix A provides simple examples of the resulting CQL code for an allergy decision node, a drug susceptibility node, a therapy choice node, and a therapy ingredient node.

In certain situations, it is possible that a concept within the comprehensive graph may not directly translate into CQL or into another executable language. By way of example, CQL may not represent contraindications well, in that case, the generator may rely upon the FHIR which is able to indicate if a drug was contraindicated or not, or, there may also be a link from the code to FDB which maintains a vast database of possible patient states and drug contraindications for each. We can query FDB with an input patient state and the drug in question and find out whether the drug is contraindicated or not. Once the executable CQL code has been generated it is output 879 to a suitable user interface 80d, to a suitable application, computer, database or for viewing by a user. The requesting entity may then use this CQL code to run against its patient data.

Above, the language CQL has been chosen because it is common and runs on FHIR, but code generator 870 may also be arranged to generate a representation of a guideline into other executable languages such as Python and SQL. Appendix B provides sample CQL code, Python code and SQL code, respectively, for fetching all therapy ingredient options prescribable when a patient does not have penicillin allergy.

In a sixth use case, the platform generates an FHIR value set 889 based upon a request 884 from a user using computer 80e (e.g., using the Value Set Generation interface of console 80) for certain nodes from the comprehensive graph. A hospital, clinic, clinician or other entity wishes to obtain this value set in order to analyze adherence with respect to standard guidelines, to monitor quality measures as prescribed by the CDC and to perform custom analytics. The request may be for any set of information within the knowledge graph, such as “all EDFs of a particular condition,” “all EDIs of a particular condition,” “all therapy ingredients, risk factors and differential conditions for a particular condition,” “all conditions that have ‘fever’ as a diagnostic factor,” “all ETIs that treat a particular condition,” “all conditions that can be treated with this ETI,” etc.

Using one very simple example of FIG. 2, if the request is for “all diagnostic factors for condition 340 including factors of any differential diagnoses,” the result 889 includes factor 356, factor 310 and factor 312. Specifically, result 889 returns an FHIR value set that includes a unique code for each requested node such as its SNOMED code. The codes in this value set may then be used in CQL code that the entity has written or in the CQL code generated using code generator 870. Below we showcase an exemplar value set for all drugs related with treatment of strep pharyngitis.

Once the value set generator has received this request 884 in natural language, it generates a query 886 to graph service 52 using an internal graph query processor. Output 888 contains the raw value set data which is then formatted by value set generator 880 into the FHIR value set requested. Appendix C provides an example of a natural language user request 884 for particular nodes (e.g., “all drugs of penicillin class used to treat strep.”) and how the value set generator processes that to form the appropriate query 886 to the graph service.

In addition, request 884 may also ask for descendent or ancestor nodes within the hierarchy of the comprehensive graph. This reasoning or inference (performed within a hierarchy such as SNOMED) is useful because FHIR computing cannot perform inference or reasoning. By way of example, if the user were to request all diagnostic factors and their ancestors for a particular condition and one of those factors is “persistent cough,” then the result would return not only a value set including “persistent cough,” but also the code for “cough.” Conversely, if the request is for diagnostic factors and their descendants, a match to “persistent cough” would also return the code for “persistent dry cough,” but not code for “cough.” Thus, any request may ask for no reasoning or inference to other diagnostic factors (or to other investigations, conditions, medications, etc.), or may ask for other ancestors or descendants or both.

Therefore, the request may simply be to match exactly the diagnostic factors for a particular condition or to match all diagnostic factors that may be inferred, e.g., “return all diagnostic factors for COVID-19” or “return all diagnostic factors for COVID-19 or that may be inferred for COVID-19.” In the case of other hierarchies or ontologies such as LOINC, the request may also include a request for sibling concepts as well. In addition, the request may be for negated factors. By way of example, if the request is for “all diagnostic factors in the hierarchy above those diagnostic factors for a particular condition,” the result would be the negated factors for this condition. Another example for negated factors is if a condition is associated with the negation of a symptom, then all hierarchical ancestors of that symptom would also negate the symptom. Thus, if the condition is associated with an absence of “persistent dry cough,” then having the absence of “cough” infers the absence of “persistent dry cough.” Similarly, the request may be for all diagnostic factors below those diagnostic factors for a particular condition in the hierarchy.

This sixth use case may be used on its own to obtain the value sets needed by a user, and it may also be used with the fifth use case above where the entity requests executable CQL code. In other words, typically an entity requesting executable CQL code 879 will also request particular FHIR value sets 889 for their specific use case.

A seventh use case does access patient state service 60 or an EHR. Such a request fetches patient data using VSG 880, for instance fetching all patients that went through a particular treatment, fetching all patients that did not satisfy a quality measure, all patients who took penicillin, etc. The value set generator is used to retrieve values related to a query from the knowledge graph; these values are then used query patient state service 60 for desired patient data. By way of example, the user queries VSG 880 with the query “return for me all patients who took drugs for strep.” Next, the VSG processes that query and formulates the appropriate query 886 to the graph service to find all codes of drugs used to treat strep, such as by finding all ETIs linked to that condition, or by finding all therapy ingredient option nodes linked to that condition and then using an ontology link to obtain the code. These codes are returned 888 to the VSG which then queries patient state service 60 with these codes to identify those patient records that contain one or more of these codes. This patient data (e.g., a patient identifier for each relevant record) is returned 887 to the user interface. Query 884 may be any query for any values of the knowledge graph, which then may be used to query patient state service 60 for any data of the patient record such as patient identifiers, drugs or other therapies used, conditions, etc. Furthermore, VSG 880 may also query an EHR or EHRs of a database instead of patient state service 60.

### Flow Diagram for Use of the Comprehensive Graph

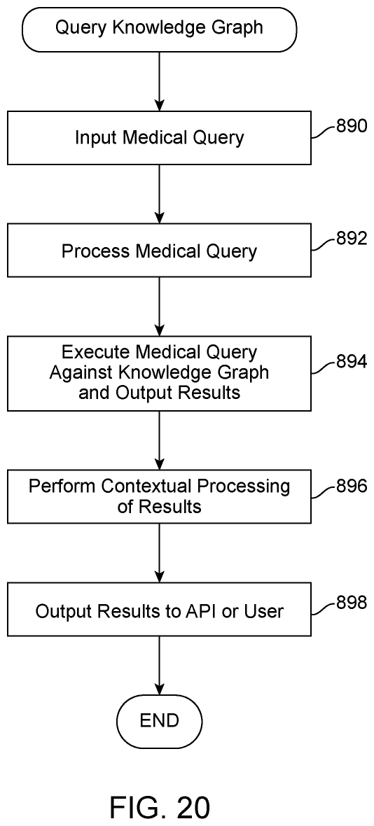

FIG. 20 is a flow diagram describing how the comprehensive graph may be queried by any of a variety of users and applications, for any of a variety of purposes, and for outputting results in a variety of forms. In step 890 a medical query is input into a computing platform destined for the comprehensive graph of the computing platform which may be implemented as shown in FIG. 1. Depending upon the query, the flow may make use of only nodes in the clinical knowledge graph, only nodes in one or more computational guidelines, or a combination of both.

Any of a wide variety of medical queries may be input including those related to patient diagnosis or treatment (or both), diagnosis by treatment, a patient population analysis, a cohort analysis, a prediction of a future patient trajectory, a claims-related query from an insurance company, a request to generate CQL executable code or code of another executable computer language, a request to return a value set from the knowledge graph, etc., all of which are described herein. As shown herein, the query may originate from a clinician using a computer, telephone or microphone, from an LLM, from a chat application, from a computer, from a software application, from an API, etc.

As shown in FIG. 5A or FIG. 6A, the query may be an indication of a known or suspected medical condition and a request to follow a computational guideline in order to diagnose or treat the patient; as shown in FIG. 6B the query may be a known medical condition and request to follow a computational treatment guideline; as shown in FIGS. 15A and 15B the query may be an input patient state and a request for diagnosis or treatment; as shown in FIG. 15A the query may be to predict a trajectory of a patient; as shown in FIG. 15B the query may be to fetch records of patients with trajectories that follow a certain trajectory; or as shown in FIG. 19 the query may be to generate executable CQL code, to return value sets or may be a query from a user filtered through an LLM.

In step 892 the medical query is processed depending upon the use case to put the query into the appropriate graph query language expected by graph service 52 of the comprehensive graph. Depending on the use case, the medical query may be processed with one or more of the following: NBA Engine 76, Chat engine 73, Trajectory engine 71, graph query processor 862, code generator 870 or value set generator 880. In the case of the flow of FIGS. 14A and 14B in the context of FIGS. 5A, 5B, 6A and 6B, the patient condition (or conditions) is processed by NBA Engine 76 to put it into the graph query language expected by the graph service. By default, NBA Engine 76 can assume that the goal is to follow a diagnosis or treatment guideline, or the goal may be specified in the request. In the case of the flow diagram of FIG. 15A in the context of the insurance company request of FIG. 18, trajectory engine 71 processes the initial patient condition to put it into the language expected by the graph service. In the case of a user query via LLM 860 of FIG. 19, graph query processor 862 takes the output from LLM 860 and places it into the correct graph query language.

In the example below the user queries the LLM in natural language to find certain medications from the knowledge graph; the LLM generates a Python query and sends it to the graph query processor to execute the query against the knowledge graph. The user input to the LLM may be “Fetch me all drugs I can prescribe to a patient suffering from strep but has reported severe allergy to penicillin.” The LLM output sent to graph query processor 862 is:
def extract_sever_pen_allergy_drugs(treatment_graph=“Strep Pharyngitis):
   #Find the no penicillin allergy node
   severe_allergy_node=next ((node for node, data in treatment_graph.nodes(data=True) if data[‘data’].name==“Severe penicillin allergy”), None)
   #Traverse graph from no allergy node to find all medication nodes
   result=[ ]
   for node in nx.descendants(treatment_graph, severe_allergy_node):
      node_data=treatment_graph.nodes[node][‘data’]
      if hasattr(node_data, ‘Therapy IngredientOptions’):
         result.extend(node_data)
   return result.

The graph query processor 862 response to the LLM after executing the query against the knowledge graph is: “{Therapy Choice: Macrolides, Therapy Ingredient Options: [Clindamycin, Azithromycin, Clarithromycin]}.” The LLM's natural language response to the user after processing the graph query processor response is “For a patient with severe Penicillin allergy it is advised to prescribe to them Macrolides such as Clindamycin, Azithromycin, or Clarithromycin.”

In the case of the query of FIG. 19 from computer 81d to generate CQL code, API 872 fields this natural language request and processes it to produce query 876 in the language expected by graph service 52. The user input may be: “Fetch me all drugs I can prescribe to a patient suffering from strep but has reported severe allergy to penicillin.” And the code generator query 876 to the graph service 52 is:
define “SevereAllergicTreatment”:
   exists (“Strep Pharyngitis Diagnosis”)
      and “Severe Penicillin Allergy”
      and exists(
         [MedicationRequest: medication CodeableConcept in “NonAllergicStrepMedications”] M where M.status in {‘active’, ‘completed’}
         and FHIRHelpers ToDateTime(M.authoredOn) during Interval[Today( )~30 days, Today( )] )

In the case of the query of FIG. 19 from computer 80e to return a value set, the value set generator 880 fields this request for the codes of a defined set of nodes within the knowledge graph and produces an input suitable for the graph service. Appendix C provides an example in which the user input is “Find me all penicillin drugs that can treat strep. pharyngitis” and the query 886 from the value set generator is shown.

Next, in step 894 the medical query is executed using the comprehensive graph and the result output. This execution may involve any combination of traversing the knowledge graph, searching the graph, reasoning using the graph or any linked ontology, matching to patient data, filtering, etc. In the case of the flow of FIGS. 15A and 15B in the context of FIGS. 5A, 5B, 6A, 6B, NBA Engine 76 may perform this execution. In the case of FIG. 15A trajectory engine 71 performs this execution. In the case of the request from LLM 860 in FIG. 19, graph query processor 862 performs this execution. In the case of the embodiments of FIG. 19, code generator 870 or value set generator 880 performs this execution as the case may be.

Next, in step 896 contextual processing is performed upon the query results. Depending upon the source and context of the query, the results may be composed with any application-specific information, the patient state, the chat or LLM history, etc. In case of the flow diagram of FIGS. 15A and 15B, the result may be composed with a determined (or absent) patient condition, a particular therapy ingredient, a probability, or any other aspect of the patient state. During the course of a diagnosis or treatment, any output query regarding a factor or investigation may also be composed with any aspect of the patient state. In the case of FIG. 15A the result may be composed with any graphical display of a portion of the knowledge graph to show probable paths, probabilities and descendent nodes, along with any aspect of the patient state. In the case of FIG. 19 the LLM 860 may compose the result it receives into a natural language response for the user in the context of any history in that interaction with the user. In the case of CQL code generation of FIG. 19, code generator 870 will take the representation it receives from the knowledge graph and generate the corresponding executable CQL code as has been discussed above. In the case of the value set generation of FIG. 19, the value set generator upon receiving output 888 will compose that output into an FHIR value set 889 for return to user interface 80e, to a computer, to an application, to an API, etc.

Finally, in step 898 the query result in the proper context is returned typically to the entity which submitted the query, e.g., an API, user interface, software application, etc. In some circumstances if a user had not generated the initial query directly, the query result in the proper context may be held in a database, database cache, etc. until a user requests it.

### Connecting the Knowledge Graph and Computational Guidelines

As described above, nodes of a clinical knowledge graph (such as that shown in FIG. 2) may be linked to or connected to nodes of a computational guideline (such as that shown in FIGS. 5A, 5B) in order to provide synergism. Examples of such connections are shown in FIGS. 2, 5A, 6A, 6B, 8-12, and 21. In general, a clinical knowledge graph includes nodes of different medical concepts linked together (e.g., conditions to diagnostic and risk factors, conditions to investigations, and conditions to therapies). Such a knowledge graph allows one to perform various actions based upon these nodes (such as determining patient state, determining next-best actions) using patient data irrespective of the chronological order of this patient data. Further, the knowledge graph itself has no inherent ordering and may be traversed from any node in any direction.

On the other hand, a computational guideline includes nodes that enable a medical guideline for a condition to be made computational, such as decision nodes, result nodes, therapy choices nodes, therapy option nodes, etc. Time intervals between actions can also be represented. A computational guideline allows us to perform certain time-dependent or “longitudinal” actions such as diagnosis, recommending a treatment, predicting a patient trajectory, comparing trajectories, and other use cases all described above, by traversing the guideline in a particular order. While one can traverse the nodes of a knowledge graph, there are no nodes that direct how a diagnosis, recommendation, computation, execution or flow should proceed. By the same token, a computational guideline does not include nodes that hold all of the factors, investigations (and results) and therapies associated with a particular condition (i.e., EDFs, EDIs, ETIs and their related information).

Connecting a knowledge graph with one or more computational guidelines is advantageous because the actions that each performs can depend upon one another and information gathered by each can be transferred from one to the other. In a first example we map time-dependent data of a patient (such as patient insurance claims over time) onto a computational guideline for a condition in order to infer static patient data (such as allergies, medications taken, investigation results, known diagnostic and risk factors), and then we apply this inferred patient data to the knowledge graph in order to perform knowledge graph actions such as determine patient state, determine next-best action, etc. In a second example, we map patient data (such as that found within an EHR) onto the knowledge graph in order to obtain likely medical conditions for the patient (e.g., using a risk score per condition), and then we use a computational guideline to predict a patient trajectory for each of these likely medical conditions obtained from the knowledge graph.

Using these two examples we can perform more complex reasoning. Using the first example, we first project longitudinal patient data onto a computational guideline in order to infer any EDFs, EDIs or ETIs of the patient. Next, using the knowledge graph, we use these inferred EDFs, EDIs or ETIs to predict the patient's likely medical conditions (or risk scores for each). Then (using the second example), we can predict a patient trajectory for each predicted medical condition with a high risk score using one or more computational guidelines, many of the inferred EDFs, EDIs and ETIs coming from the first example.

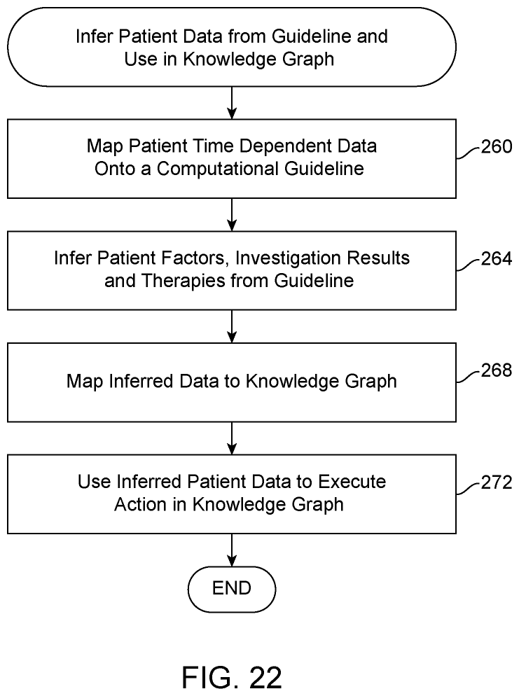

FIG. 22 is a flow diagram describing how patient data inferred from a guideline may be used in a knowledge graph action. In a first step 260, any time-dependent patient data is mapped onto a computational guideline for a given condition. Such data may include any medical actions over time, insurance claims data, EHR patient data with timestamps, clinical trial data, etc. Starting with a guideline for a known or suspected condition of the patient, we traverse the guideline using the known patient data to help us infer other unknown patient data that is not currently present in the patient state, in the EHR or in other source of the current patient data. In one example, we use codes in the current patient data (such as CPT, ICD codes or other) to look up and match with decision nodes, decision result nodes, therapy choices nodes, therapy ingredient nodes, or determination nodes in the guideline to determine the path the patient has taken.

Which path a patient has taken from a decision node, or even which nodes have been skipped, allow us to infer patient data in step 264. By way of example, knowing that the patient has taken penicillin means that the patient is not allergic to penicillin and that the patient has the EDF “not allergic to penicillin.” Or, knowing that the patient has taken a macrolide allows us to infer that the patient is allergic to penicillin and thus has the EDF “allergic to penicillin.” Knowing that the patient has taken hydroxychloroquine allows us to infer that his CDAI score was “low activity” and thus he has an EDI result of “CDAI score is low activity.” Knowing that the patient is being treated for rheumatoid arthritis and knowing that he now has retinopathy (after a gap in time), we can infer that the patient has taken hydro chloroquine and thus has the ETI of “hydro chloroquine.” Thus, we can infer EDFs, EDI results and ETIs based upon a path the patient data traverses in a computational guideline.

One may make a given inference based exclusively upon information within the guideline or may traverse using a link to the knowledge graph to make an inference using nodes of that graph. The approach depends upon whether one wishes to make an inference about the same medical condition of the guideline or about another medical condition. By way of example, for an intra-condition inference using the computational guideline is preferred. Knowing that the patient chose a particular path from node 408 of the guideline regarding a penicillin allergy is an important decision node and we can infer with a higher confidence knowing which path the patient traversed (e.g., severe allergy, no allergy or mild allergy). In a second example, for an inter-condition inference we are traversing the guideline of the first condition in order to infer other medical concept associated with other conditions; traversing to the knowledge graph from a link in the guideline and then traversing upwards through parent nodes from EDFs, EDIs and ETIs to make an inference is the preferred approach, as the guideline may only contain links to EDFs, EDIs and ETIs associated with its first condition.

In a next step 268, this inferred patient data is then mapped to a knowledge graph by saving the patient data into a patient state service associated with a knowledge graph, by flagging knowledge graph nodes that correspond to the newly inferred patient data, or by using techniques described in steps 802-806, or in U.S. Pat. No. 11,869,674. Next in step 272, we use this newly inferred patient data along with any existing patient data from the patient state service to perform an action using the knowledge graph such as determining the patient state, determining the next-best action, etc. Thus, we may use a clinical knowledge graph to determine other likely conditions that the patient may have or desired next-best actions based upon traversal of a computational guideline.

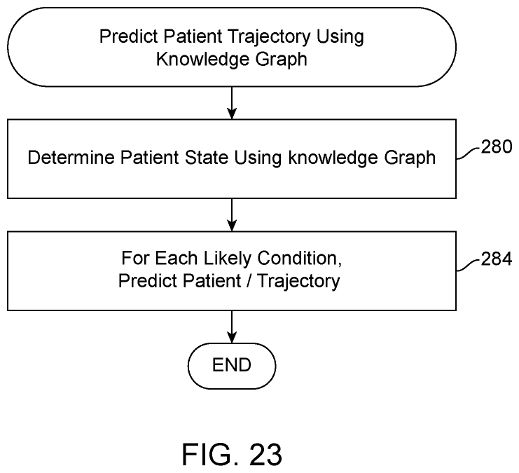

FIG. 23 is a flow diagram describing how to predict a patient trajectory using information obtained from a knowledge graph. As mentioned above, the results of various actions performed using the knowledge graph may then be used in a computational guideline. For instance, FIG. 16 describes how to use a patient medical condition with a computational guideline. In a first step 280, patient data is determined using the knowledge graph such as by determining the patient state or determining a next-best action using techniques described in U.S. Pat. No. 11,869,674, in attorney docket Nos. RCVRP009P, P009A, P009B and P009C, or as herein described in order to obtain a list of likely patient medical conditions, likely or known factors, investigation results or therapies. Next, in step 284 for each likely medical condition of the patient, we predict a patient trajectory for that medical condition using the steps of FIG. 16. The result is a prediction for the patient using a computational guideline that has been based upon a likely medical condition obtained from the knowledge graph.

Above are presented a variety of embodiments for querying a clinical knowledge graph, querying a computational guideline, querying a comprehensive graph that includes a clinical knowledge graph and any number of computational guidelines, determining a diagnosis or treatment for a patient, predicting or comparing patient trajectories, for performing any number of use cases, and for using inferred data from a guideline in a clinical knowledge graph and vice versa. Each of these may generate an output for a user, whether a final output or a recommendation, or a prompt back to the clinician to perform an observation, investigation or treatment. Computational platform 10 and the other hardware and software shown herein is arranged to generate such an output, recommendation or prompt in under one second, in some cases within a few seconds, in some cases under one minute, or on the order a few minutes, or in less than five minutes, in less than ten minutes, in less than one-half hour, in less than one hour, or in less than any other quantity of minutes, depending upon the computational power used.

Patient State and Patient Trajectory Prediction Using Inference from Multiple Data Sources

A further embodiment captures that patient diagnostic factors, investigation results, and patient state can be inferred from multiple data sources (e.g., EHR, insurance claims data, clinical trial data) via computational guideline traversal, ontology reasoning, and probabilistic modeling, rather than being directly observed (e.g., from clinician-entered data).

Regarding “probabilistic modeling,” the above describes probabilistic reasoning, probabilities and likelihoods, rankings and weights, and that the NBA engine uses a probabilistic model, all in support of inference using probabilistic modeling. More specifically, above we describe that: EDFs/EDIs/ETIs are computational because they can be used in “probabilistic reasoning;” and that group nodes include how an element is probabilistically related to a condition, which enables probabilistic calculations and inferences. We also describe how: factor groups can store a ranking and a weight value; that weight can incorporate sensitivity, specificity, and likelihood ratio; that the ranking expresses how likely a factor group is caused by or causes a condition; and that the weight can be calculated from those probability-related measures. We also explain how results may include a probability, and graphical displays may show probable paths and probabilities. Regarding therapies in particular, we provide another probability-style formulation: a therapy group ranking indicates how often an ETI is used for a linked condition (for example, 90% plus of cases, 30-90%, etc.). This is another example of encoding probabilistic or frequency-based relationships in the graph.

Thus, a probabilistic model may be a model that the system uses to assign and update probability values for possible diagnoses or explanations, based on patient context and graph relationships. Probabilistic modeling is the way the system encodes and uses likelihood-related information in the knowledge graph-through rankings, weights, sensitivity, specificity, likelihood ratios, and probabilistic links between elements and conditions.

This embodiment is also able to use other data sources. For instance, we describe above that the Patient State Service 60 ingests patient data from diverse sources and in diverse format and transforms that data into internal system-specific representations, while the NBA Engine can use structured sources plus extracted concepts from unstructured data. Also, the platform can combine computational guidelines with other healthcare data (including patient data and other real-world data), in context and in real time. Other data sources include: wearable sensor data, genomic data and remote patient monitoring.

Wearable sensor data is patient-generated physiologic and activity data collected continuously or intermittently by consumer or medical wearables, such as: heart rate, heart-rate variability, oxygen saturation, skin temperature, respiratory rate, sleep metrics, activity level, gait, arrhythmia alerts, glucose readings from CGMs, and similar signals. These may be input as structured, time-stamped observations through the heterogeneous-ingestion path using Patient State Service 60. Service 60 accepts data from diverse sources and formats and keeps track of patient state over time, including factors, investigations and treatments. Wearable sensor data feeds may be normalized into internal patient-state representations and then mapped to EDFs and EDIs depending on the nature of the signal. E.g., above we describe various physiologic measurements and vital signs as investigations, especially where the result is numeric or range-based. Thus, a wearable-derived resting tachycardia, nocturnal hypoxemia, reduced activity tolerance, irregular rhythm alert, persistent fever trend, etc., may be represented as an inferred EDF and/or as an EDI with a time-stamped result node, then used by Reasoning Service and NBA Engine in the same way that EHR or claims-derived data are used.

Genomic data includes germline and somatic variant data, pharmacogenomic markers, polygenic risk signals, molecular pathology findings, gene-expression signatures, and other sequence-based or assay-based molecular results. This genomic data can be entered as structured laboratory-style data or as concepts extracted from reports, in a similar way that we describe ingesting structured patient data, extracting concepts from text, and mapping them to graph nodes via ontologies and unique codes. We also describe above investigations as laboratory tests with numeric, categorical, ordinal, or binary results, and allows nondrug/genetic therapy concepts to link to suitable ontologies. Consistent with that design, genomic findings may be represented in at least three ways: as risk factors when they predispose to conditions; as investigation results when they are assay outputs relevant to diagnosis or prognosis; and as treatment-selection constraints when they affect therapy eligibility, contraindications, or dosing. Also, our distinction between elemental nodes and condition-specific properties fits genomics well: the molecular concept can remain atomic, while its condition-specific significance, ranking, restriction, or treatment relevance can be stored in linked contextual nodes or edges.

Remote patient monitoring (RPM) data refers to data collected outside the clinic from home or community monitoring devices and transmitted to the care platform, such as blood pressure cuffs, home pulse oximeters, weight scales, spirometers, ECG patches, home INR devices, symptom check-ins, and clinician-prescribed chronic-disease monitoring streams. In practice, RPM overlaps with wearables, but it usually implies a more clinical and supervised monitoring workflow. RPM data may be another external source feeding Patient State Service 60, much like EHR data, claims data, or manual clinician/patient inputs. Above, we emphasize time-dependent patient data and repeated monitoring over time, including delays, follow-up testing, and updates to patient state. This means that RPM is especially compatible the platform: periodic home measurements can populate time-stamped investigation results, trigger follow-up decision nodes, update patient trajectory state, and support next-best-action recommendations when thresholds, worsening trends, or missing follow-up events are detected.

These new data sources may be input into the platform as follows. The platform receives wearable, genomic, and RPM data through external APIs, file imports, device integrations, payer/provider integrations, or user-entered/clinician-reviewed interfaces, as additional diverse sources and diverse formats handled by Patient State Service 60. The incoming data are normalized into the platform's internal representation, including timestamps, source metadata, units, confidence/provenance, and coding to suitable ontologies or unique identifiers, all consistent with our ontology-linked elemental nodes. Structured values are mapped directly to EDFs, EDIs, EDI results, ETI restrictions, risk factors, or other patient-state elements; unstructured device reports or genomics narratives may be processed by NLP Engine 74 and then mapped to graph nodes. Reasoning Service 72 and NBA Engine 76 then use these mapped concepts, along with existing EHR data, insurance claims data and/or patient chart data, to infer additional factors, investigations, treatments, likely conditions, and next-best actions. The resulting inferred and observed items are stored in the patient state and can be used in guideline traversal, diagnosis, treatment selection, and trajectory prediction as described in the flows above.

### FURTHER EMBODIMENTS

Any of the method claims listed below may be embodied as an apparatus claim for performing the steps of that method claim that includes a computer system comprising one or more processors and memory that stores instructions, wherein the instructions, when executed by the one or more processors, cause the one or more processors to execute the steps of that method claim. Further, any method or apparatus claim as recited wherein a total number of nodes in the knowledge graph is at least 20,000 nodes. Yet further, any method or apparatus claim as recited wherein a total number of nodes in the knowledge graph is at least 50,000 nodes. And yet further, any method or apparatus claim as recited wherein a total number of nodes in the knowledge graph is at least 100,000 nodes.

Further included is this embodiment of claim 1. A method as recited in claim 1, wherein said diagnostic factor node is an elemental diagnostic factor node that is atomic and independent of any particular medical condition and is linked to a plurality of medical condition nodes in said knowledge graph without storing medical condition-dependent properties within said elemental diagnostic factor node, wherein said matching comprises matching said known or unknown diagnostic factor of said patient to said elemental diagnostic factor node using an ontology link within said elemental diagnostic factor node that references said diagnostic factor within a clinical ontology, and wherein said continuing said traversing further comprises arriving at a second decision node of said guideline nodes, and from said second decision node, traversing to a second node of said knowledge graph that is linked to said medical condition node, said second node being at least one of an elemental diagnostic factor node, an elemental investigation node, or an elemental therapeutic intervention node.

Further included is this embodiment of claim 11. A method as recited in claim 11, wherein said investigation node is an elemental investigation node that is atomic and independent of any particular medical condition and is linked to a plurality of medical condition nodes in said knowledge graph, wherein a result specific to said suspected patient condition is stored in a result node separate from said elemental investigation node, wherein said matching comprises matching said investigation result of said patient to said elemental investigation node and to said result node as a pair using an ontology link within said elemental investigation node that references said investigation within a clinical ontology, and wherein said method further comprises, based upon said matching, traversing from said elemental investigation node to at least one medical condition node other than said medical condition node of said suspected patient condition to identify at least one additional medical condition indicated by said investigation result.

Further included is this embodiment of claim 16. A method as recited in claim 16 wherein said nondrug therapy node links to an elemental therapeutic intervention node in said knowledge graph that is atomic and independent of any particular medical condition and represents a prescribable form of said nondrug therapy, wherein said elemental therapeutic intervention node is linked to a plurality of medical condition nodes through respective therapy restriction nodes that store condition-dependent restrictions separate from said elemental therapeutic intervention node, wherein said recommendation is based in part upon information obtained from said elemental therapeutic intervention node or from at least one ancestor node of said elemental therapeutic intervention node, and wherein said continuing said traversing comprises, at a subsequent decision node of said guideline nodes, using information from said elemental therapeutic intervention node or said at least one ancestor node to inform a subsequent decision in said traversal of said medical guideline.

Further included is this embodiment of claim 27. A method as recited in claim 27, wherein said child node of said drug therapy node is a therapy ingredient option node that links to an elemental therapeutic intervention node in said knowledge graph, said elemental therapeutic intervention node being atomic and independent of any particular medical condition and representing a prescribable form of said medication, wherein said elemental therapeutic intervention node includes an ontology link to a drug ontology, wherein said recommendation is based in part upon a score calculated for said therapy ingredient option node by comparing a patient state of said patient to at least one of a therapy restriction node linked to said elemental therapeutic intervention node or information obtained via said ontology link, and wherein said continuing said traversing comprises traversing to a dosage decision node that calculates a dosage regimen for said patient based upon said patient state and a rule link associated with said dosage decision node, followed by traversing to a subsequent decision node that monitors a therapeutic response of said patient to said medication.

Further included is this embodiment of claim 27. A method as recited in claim 27 further comprising: traversing from one of said guideline nodes to a therapeutic intervention node in said knowledge graph and determining a result found within said therapeutic intervention node or in an ancestor node of said therapeutic intervention node; and outputting a recommendation based in part upon said result.

### Embodiments for Technology Improvements

Prior art computer systems for clinical decision support represent medical guidelines using condition-specific rule sets, static document-based rules, or executable scripts designed for a single context (such as CQL). In these prior art systems, clinical factors, investigations, and therapies are encoded with condition-dependent properties embedded within each element, requiring duplication of the same clinical concept across multiple conditions and preventing computational reuse across conditions. This duplication increases storage requirements, increases the number of database queries needed for cross-condition analysis, and prevents a single traversal operation from reasoning across conditions. Embodiments of the present invention addresses these technical limitations through a specific graph architecture in which elemental nodes (EDFs, EDIs, ETIs) are atomic and independent-stripped of condition-dependent properties, which are instead stored in separate contextual nodes or edges linked to specific conditions- and in which a clinical knowledge graph ontology is structurally connected to a computational guideline ontology. This connected architecture enables a computer to perform a cross-ontology traversal in a single computational operation, retrieving information from the clinical knowledge graph that is not present within the guideline, and returning that information to the guideline to continue execution. This specific architecture reduces redundant storage of clinical concepts, reduces the computational complexity of cross-condition reasoning, and enables the computer system to process medical queries involving thousands of nodes and probabilistic relationships in timeframes that are computationally infeasible using prior art data structures.

By making each elemental node (EDF, EDI, ETI) atomic, independent, and free of condition-dependent properties, the computer system achieves several technical advantages. First, a single canonical node can represent a clinical concept (such as ‘fever’ or ‘penicillin V’) used across multiple conditions, eliminating the need to store duplicate representations and reducing the storage footprint of the knowledge graph. Second, patient data can be matched to a single canonical node rather than to multiple condition-specific duplicates, reducing the number of matching operations required. Third, because each elemental node includes an ontology link to a standardized clinical ontology, the computer system can perform inferential reasoning by traversing the ontology hierarchy from that node-expanding or narrowing the clinical concept-without requiring separate lookup databases or additional computational pipelines. These technical advantages are a direct consequence of the specific data architecture described herein and are not achievable by prior art systems that embed condition-dependent properties within clinical concept representations.

Connecting a clinical knowledge graph with a computational guideline ontology produces a comprehensive graph that enables computational operations that neither ontology can achieve independently. When a guideline decision node links to an elemental node (EDF, EDI, or ETI) in the clinical knowledge graph, the computer system can, during a single guideline traversal operation, access all conditions linked to that elemental node, all restrictions associated with the elemental node's therapy group, and all ancestor-descendant relationships available through the elemental node's ontology link-information that is not encoded within the guideline itself and that would be unavailable to any system that represents the guideline in isolation. This cross-ontology traversal constitutes a specific computational operation enabled by the structural linkage between the two ontologies and produces inferred data—such as cross-condition allergies, therapy contraindications, or additional diagnostic factors—that improves the accuracy and completeness of the system's output. As a result, the comprehensive graph reduces the number of separate queries required for cross-condition reasoning compared to prior art systems that maintain separate databases for clinical knowledge and guideline logic.

By virtue of the graph architecture described herein, computational platform 10 can process a medical query involving a knowledge graph of at least 10,000 or more nodes, match patient data to elemental nodes, perform inferential reasoning using ontology hierarchies, and traverse a computational guideline to produce a diagnosis, treatment recommendation, or predicted trajectory, all within seconds to minutes depending upon the computational power used. As described above, this same process performed manually by a human physician would require days to weeks per patient with error rates exceeding 50%. The specific data architecture—in which elemental nodes are atomic and independent, condition-dependent properties are held in separate contextual nodes, and clinical knowledge and guideline ontologies are structurally connected—is what enables this computational performance; prior art systems using condition-specific rule sets, document-based guidelines, or isolated CQL scripts cannot achieve comparable performance because they lack the structural connections that enable cross-condition reasoning in a single traversal.

### Computer System Embodiment

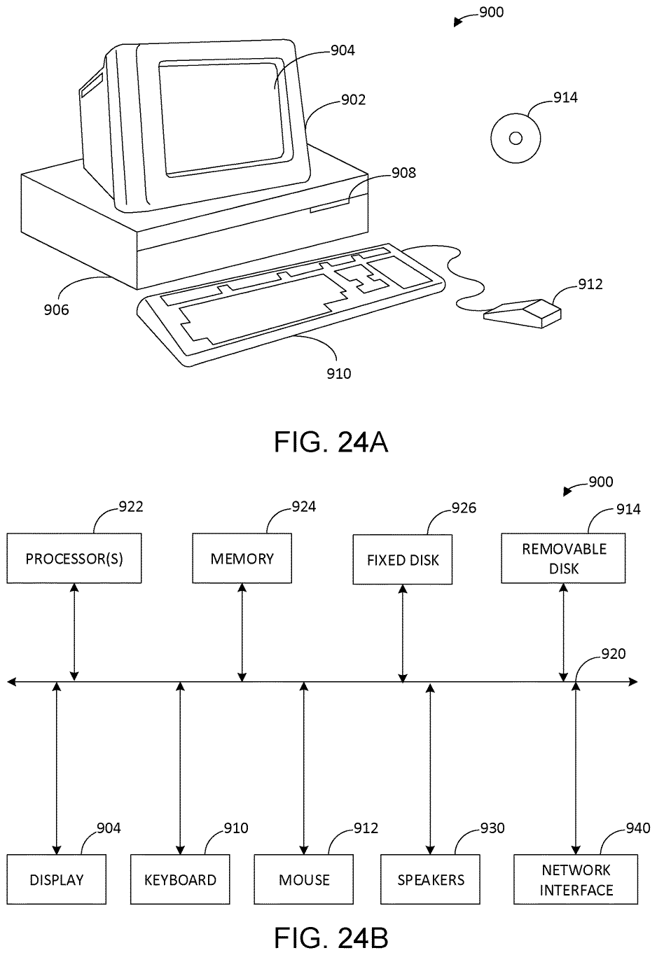

FIGS. 24A and 24B illustrate a computer system 900 suitable for implementing embodiments of the present invention. FIG. 24A shows one possible physical form of the computer system. Of course, the computer system may have many physical forms including an integrated circuit, a printed circuit board, a small handheld device (such as a mobile telephone or PDA), a personal computer or a super computer. Computer system 900 includes a monitor 902, a display 904, a housing 906, a disk drive 908, a keyboard 910 and a mouse 912. Disk 914 is a computer-readable medium used to transfer data to and from computer system 900.

FIG. 24B is an example of a block diagram for computer system 900. Attached to system bus 920 are a wide variety of subsystems. Processor(s) 922 (also referred to as central processing units, or CPUs) are coupled to storage devices including memory 924. Memory 924 includes random access memory (RAM) and read-only memory (ROM). As is well known in the art, ROM acts to transfer data and instructions uni-directionally to the CPU and RAM is used typically to transfer data and instructions in a bi-directional manner. Both of these types of memories may include any suitable of the computer-readable media described below. A fixed disk 926 is also coupled bi-directionally to CPU 922; it provides additional data storage capacity and may also include any of the computer-readable media described below. Fixed disk 926 may be used to store programs, data and the like and is typically a secondary mass storage medium (such as a hard disk, a solid-state drive, a hybrid drive, flash memory, etc.) that can be slower than primary storage but persists data. It will be appreciated that the information retained within fixed disk 926, may, in appropriate cases, be incorporated in standard fashion as virtual memory in memory 924. Removable disk 914 may take the form of any of the computer-readable media described below.

CPU 922 is also coupled to a variety of input/output devices such as display 904, keyboard 910, mouse 912 and speakers 930. In general, an input/output device may be any of: video displays, track balls, mice, keyboards, microphones, touch-sensitive displays, transducer card readers, magnetic or paper tape readers, tablets, styluses, voice or handwriting recognizers, biometrics readers, or other computers. CPU 922 optionally may be coupled to another computer or telecommunications network using network interface 940. With such a network interface, it is contemplated that the CPU might receive information from the network, or might output information to the network in the course of performing the above-described method steps. Furthermore, method embodiments of the present invention may execute solely upon CPU 922 or may execute over a network such as the Internet in conjunction with a remote CPU that shares a portion of the processing.

In addition, embodiments of the present invention further relate to computer storage products with a computer-readable medium that have computer code thereon for performing various computer-implemented operations. The media and computer code may be those specially designed and constructed for the purposes of the present invention, or they may be of the kind well known and available to those having skill in the computer software arts. Examples of computer-readable media include, but are not limited to: magnetic media such as hard disks, floppy disks, and magnetic tape; optical media such as CD-ROMs and holographic devices; magneto-optical media such as floptical disks; and hardware devices that are specially configured to store and execute program code, such as application-specific integrated circuits (ASICs), programmable logic devices (PLDs) and ROM and RAM devices. Examples of computer code include machine code, such as produced by a compiler, and files containing higher-level code that are executed by a computer using an interpreter.

Although the foregoing invention has been described in some detail for purposes of clarity of understanding, it will be apparent that certain changes and modifications may be practiced within the scope of the appended claims. Therefore, the described embodiments should be taken as illustrative and not restrictive, and the invention should not be limited to the details given herein but should be defined by the following claims and their full scope of equivalents.

### APPENDIX A—SAMPLE CQL CODE

library AntibioticTreatment version ‘1.0.0’
using FHIR version ‘4.0.1’
include FHIRHelpers version ‘4.0.1’
// Codesystems and valuesets
codesystem “SNOMED”: ‘http://snomed.info/set’
codesystem “RXNORM”: ‘http://www.nlm.nih.gov/research/umls/rxnorm’
codesystem “LOINC”: ‘http://loinc.org’
valueset “Acute Pharyngitis Treatment”: ‘http://evidium.info/edi/fhir/ValueSet/<uri>’
valueset “Penicillin Allergy Observation”:‘http://snomedct.info/set/<uri>’
valueset “Penicillins”: ‘http://rxnorm.info/rxcui/fhir/ValueSet/<uri>’
valueset “Cephalosporins”: ‘http://rxnorm.info/rxcui/fhir/ValueSet/<uri>’
valueset “Macrolides”: ‘http://rxnorm.info/rxcui/fhir/ValueSet/<uri>’
// Value sets for allergy severities
codesystem “SNOMED”: ‘http://snomed.info/set’
code “NoKnownAllergy”: ‘716186003’ from “SNOMED” display ‘No known allergy’
code “MildAllergy”: ‘255604002’ from “SNOMED” display ‘Mild’
code “SevereAllergy”: ‘24484000’ from “SNOMED” display ‘Severe’
code “UnknownSeverity”: ‘261665006’ from “SNOMED” display ‘Unknown’
context Patient
// CQL for Penicillin Allergy Decision: Each of the CQL function below returns a Boolean whenever the criteria matches
define “Active Penicillin Allergies”:
   [AllergyIntolerance: “Penicillins”] Allergy
      where Allergy.clinicalStatus~“active”
define “No Penicillin Allergy”:
   not exists “Active Penicillin Allergies”
define “Mild Penicillin Allergy”:
   exists (
      “Active Penicillin Allergies” Allergy
         where Allergy.severity~“mild”
define “Severe Penicillin Allergy”:
   exists (
      “Active Penicillin Allergies” Allergy
      where Allergy.severity~“severe”
define “Unknown Penicillin Allergy Severity”:
   exists (
      “Active Penicillin Allergies” Allergy
      where Allergy.severity is null
// CQL for Penicillin Susceptibility Testing: Returns all penicillin susceptibility observations in a descending order
define “Penicillin Susceptibility Test”:
   From [Observation: “Penicillin Susceptibility Panel”] PenTest
      where PenTest. status=‘final’
      and PenTest.value!=null
   return PenTest
      sort by PenTest.effectiveDateTime desc
define “Latest Penicillin Susceptibility”:
   First (“Penicillin Susceptibility Test”)
define “Penicillin Susceptible”:
   “Latest Penicillin Susceptibility” result=“SUSCEPTIBLE”
define “Penicillin Resistant”:
   “Latest Penicillin Susceptibility”.result=“RESISTANT”
// CQL for therapy choices & ingredients: Each CQL function below returns boolean for whether a patient satisfy the treatment criteria or not
// Treatment Eligibility Definitions
define “Penicillin Treatment Eligible”:
   “No Penicillin Allergy” and
   (“Penicillin Susceptible” or “Penicillin Susceptibility Unknown”)
define “Cephalosporin Gen1 Treatment Eligible”:
   (“Mild Penicillin Allergy” or
   (“No Penicillin Allergy” and “Penicillin Resistant”)) and
   (“Cephalosporin Gen1 Susceptible” or not exists “Latest Cephalosporin Gen1 Susceptibility Test”)
define “Cephalosporin Gen2 Treatment Eligible”:
   (“Cephalosporin Gen1 Treatment Eligible” and “Cephalosporin Gen1 Resistant”) or exists (
   [Condition] C
      where C.code in “Cephalosporin Gen1 Contraindications”
      and C.clinicalStatus~“active”
define “Cephalosporin Gen3 Treatment Eligible”:
   “Cephalosporin Gen2 Treatment Eligible” and “Cephalosporin Gen2 Resistant”
define “Macrolide Treatment Eligible”:
   “Severe Penicillin Allergy” or
   “Unknown Penicillin Allergy Severity” or
   (“Cephalosporin Gen3 Treatment Eligible” and “Cephalosporin Gen3 Resistant”)

### APPENDIX B—SAMPLE CQL, PYTHON AND SQL CODE

define “AppropriateNonAllergicTreatment”:
   exists (“Strep Pharyngitis Diagnosis”)
      and “No Penicillin Allergy”
      and exists (
         [MedicationRequest: medicationCodeableConcept in “NonAllergicStrepMedications”] M where M.status in {‘active’, ‘completed’}
         and FHIRHelpers. ToDateTime (M.authoredOn) during Interval[Today ( )−30 days, Today( )]
def extract_no_pen_allergy_rxcui_codes (treatment_graph):
   #Find the no penicillin allergy node
   no_allergy_node=next ((node for node, data in treatment_graph.nodes (data=True) if data[‘data’]. name==“No penicillin allergy”), None)
   #Traverse graph from no allergy node to find all medication nodes
   result=[ ]
   for node in nx.descendants (treatment_graph, no_allergy_node):
      node_data=treatment_graph.nodes[node][‘data’]
      if hasattr (node_data, ‘Therapy IngredientOptions’):
         result.extend (node_data)
   return result
SELECT
   tio.id,
   tio.code,
   tio.description,
   tio.rxcui,
   tio.ingredient_name
FROM
   Therapy IngredientOptions tio
JOIN
   MedicationRequest mr ON mr.medication_code=tio.code
JOIN
   Patient p ON mr.patient_id=p.id
JOIN
   Diagnosis d ON d.patient_id=p.id
LEFT JOIN
   PatientAllergy pa ON pa.patient_id=p.id AND pa.allergy_code IN (
      SELECT code FROM Allergies WHERE name LIKE ‘% penicillin %’
WHERE
   d.code IN (SELECT code FROM Diagnosis WHERE name=‘Strep Pharyngitis’)
   AND d.status=‘active’
   AND pa.id IS NULL—No penicillin allergy
   AND mr.status IN (‘active’, ‘completed’)
   AND mr.authored_on BETWEEN DATEADD (day,−30, GETDATE( )) AND GETDATE ( )
   AND tio.code IN (
      SELECT code FROM MedicationCatalog WHERE category=
‘NonAllergicStrepMedications’
ORDER BY
   tio.ingredient_name;

### APPENDIX C—SAMPLE QUERY FROM VALUE SET GENERATOR

{“resourceType”: “ValueSet”,
   “id”: “1.3.6.1.4.1.33895.1.3.0.45”,
   “meta”: {
      “versionId”: “29”,
      “lastUpdated”: “2023-12-21T17:43:03.000-05:00”,
      “profile”: [
         “http://h17.org/fhir/StructureDefinition/shareablevalueset”,
         “http://h17.org/fhir/us/cqfmeasures/StructureDefinition/computable-valueset-cqfm”,
         “http://h17.org/fhir/us/cqfmeasures/StructureDefinition/publishable-valueset-cqfm”] },
   “extension”: [
      }
         “url”: “http://h17.org/fhir/StructureDefinition/valueset-effectiveDate”,
         “valueDate”: “2021 Feb. 20”} ],
   “url”: “http://cts.nlm.nih.gov/fhir/ValueSet/1.3.6.1.4.1.33895.1.3.0.45”,
   “identifier”: [ {
      “system”: “urn:ietf.rfc:3986”,
      “value”: “urn:oid:1.3.6.1.4.1.33895.1.3.0.45”} ],
   “version”: “20210220”,
   “name”: “Penicillins”,
   “title”: “Penicillins for treating Acute Pharyngitis”,
   “status”: “active”,
   “experimental”: false,
   “date”: “2021-02-20T01:02:00-05:00”,
   “publisher”: “Evidium Medical”.
   “expansion”: {
      “identifier”: “urn:uuid:48d09229-aed2-4882-84ca-fc9ece2db06a”,
      “timestamp”: “2024-12-01T19:46:46-05:00”,
      “total”: 4,
      “offset”: 0,
      “parameter”: [ {
         “name”: “count”,
         “valueInteger”: 1000}, {
         “name”: “offset”,
         “valueInteger”: 0} ],
      “contains”: [
         {“system”:“http://www.nlm.nih.gov/research/umls/rxnorm”,
         “code”: “7980”,
         “display”: “Penicillin G”},
         { “system”: “http://www.nlm.nih.gov/research/umls/rxnorm”,
         “code”: “7984”,
         “display”: “Penicillin V” {,
         { “system”: “http://www.nlm.nih.gov/research/umls/rxnorm”,
         “code”: “723”,
         “display”: “Amoxicillin” } ] } }

We claim:

1. A method of diagnosing a patient using a computational guideline of a knowledge graph, said method comprising:
inputting a suspected patient condition and matching said patient condition to a medical condition node of said knowledge graph, said knowledge graph including a plurality of guideline nodes representing a medical guideline;
traversing to a decision node of said guideline nodes;
from said decision node, traversing to a diagnostic factor node of said knowledge graph that is linked to said medical condition node;
matching a known or unknown diagnostic factor of said patient represented by said diagnostic factor node to a child guideline node of said decision node; and
based upon said matching, continuing said traversing of said guideline nodes from said child guideline node.

2. A method as recited in claim 1 further comprising:
obtaining, from an electronic health record (EHR) of said patient or by querying said patient, said known or unknown diagnostic factor of said patient.

3. A method as recited in claim 1 further comprising:
determining a result that said suspected patient condition is present or absent in said patient and outputting said result.

4. A method as recited in claim 1 further comprising:
traversing from one of said guideline nodes to a therapeutic intervention node in said knowledge graph and determining a result found within said therapeutic intervention node or in an ancestor node of said therapeutic intervention node; and
outputting a recommendation based in part upon said result.

5. A method as recited in claim 1 wherein said knowledge graph includes at least 10,000 nodes.

6. A method as recited in claim 1 wherein at least one of said diagnostic factors, said investigation results, or said patient condition is inferred from patient data of a plurality of data sources using at least one of: inferential reasoning based on structural relationships within a computational guideline graph, hierarchical relationships in a clinical ontology, or a probabilistic model that recalculates diagnostic predictions based on said patient data.

7. A method as recited in claim 6 wherein said plurality of data sources includes at least two of: electronic health record (EHR) data, insurance claims data, clinical trial data, or clinician-entered data.

8. A method as recited in claim 6 wherein said inferred diagnostic factor, investigation result, or patient condition is associated with a recalculated diagnostic prediction produced by a probabilistic model, said method further comprising using said recalculated diagnostic prediction in said traversing or said querying.

9. A method as recited in claim 6 wherein said inferring comprises traversing ancestor-descendant relationships in said clinical ontology to identify clinical concepts broader or narrower than concepts present in said patient data.

10. A method as recited in claim 6 further comprising: receiving additional patient data from at least one of said data sources; updating said inferred diagnostic factor, investigation result, or patient condition based upon said additional patient data; and re-executing at least a portion of said method using said updated inferred data.

11. A method of diagnosing a patient using a computational guideline of a knowledge graph, said method comprising:
inputting a suspected patient condition and matching said patient condition to a medical condition node of said knowledge graph, said knowledge graph including a plurality of guideline nodes representing a medical guideline;
traversing to a decision node of said medical guideline nodes;
from said decision node, traversing to an investigation node of said knowledge graph node;
matching an investigation result of said patient represented by said investigation node to a child guideline node of said decision node; and
based upon said matching, continuing said traversing of said guideline nodes from said child guideline node.

12. A method as recited in claim 11 further comprising:
obtaining, from an electronic health record (EHR) of said patient or by querying said patient, said investigation result of said patient.

13. A method as recited in claim 11 further comprising:
determining a result that said suspected patient condition is present or absent in said patient and outputting said result.

14. A method as recited in claim 11 further comprising:
traversing from one of said guideline nodes to a therapeutic intervention node in said knowledge graph and determining a result found within said therapeutic intervention node or in an ancestor node of said therapeutic intervention node; and
outputting a recommendation based in part upon said result.

15. A method as recited in claim 11 wherein said knowledge graph includes at least 10,000 nodes.

16. A method of treating a patient using a computational guideline of a knowledge graph, said method comprising:
inputting a patient condition and matching said patient condition to a medical condition node of said knowledge graph, said knowledge graph including a plurality of guideline nodes representing a medical guideline;
traversing said guideline nodes from said medical condition node, wherein when a nondrug therapy node is reached, outputting a recommendation for said patient to perform a nondrug therapy represented by said nondrug therapy node; and
continuing said traversing of said guideline nodes from a child node of said nondrug therapy node.

17. A method as recited in claim 16 when said nondrug therapy node is reached, said method further comprising:
traversing to a therapeutic intervention node of said knowledge graph that is linked to said medical condition node; and
outputting a recommendation for said patient to perform a nondrug therapy represented by said therapeutic intervention node.

18. A method as recited in claim 16 wherein when a decision node of said guideline nodes is reached, said method further comprising:
matching a diagnostic factor of said patient or an investigation result of said patient to a child node of said decision node and continuing said traversing from said child node.

19. A method as recited in claim 16, wherein when a determination node of said guideline nodes is reached, said method further comprising:
determining a result of said medical guideline; and
outputting said result of said medical guideline.

20. A method as recited in claim 16 further comprising:
traversing from one of said guideline nodes to a therapeutic intervention node in said knowledge graph and determining a result found within said therapeutic intervention node or in an ancestor node of said therapeutic intervention node; and
outputting a recommendation based in part upon said result.

21. A method as recited in claim 16 wherein said knowledge graph includes at least 10,000 nodes.

22. A method as recited in claim 16 wherein at least one of said diagnostic factors, said investigation results, or said patient condition is inferred from patient data of a plurality of data sources using at least one of: inferential reasoning based on structural relationships within a computational guideline graph, hierarchical relationships in a clinical ontology, or a probabilistic model that recalculates diagnostic predictions based on said patient data.

23. A method as recited in claim 22 wherein said plurality of data sources includes at least two of: electronic health record (EHR) data, insurance claims data, clinical trial data, or clinician-entered data.

24. A method as recited in claim 22 wherein said inferred diagnostic factor, investigation result, or patient condition is associated with a recalculated diagnostic prediction produced by a probabilistic model, said method further comprising using said recalculated diagnostic prediction in said traversing or said querying.

25. A method as recited in claim 22 wherein said inferring comprises traversing ancestor-descendant relationships in said clinical ontology to identify clinical concepts broader or narrower than concepts present in said patient data.

26. A method as recited in claim 22 further comprising: receiving additional patient data from at least one of said data sources; updating said inferred diagnostic factor, investigation result, or patient condition based upon said additional patient data; and re-executing at least a portion of said method using said updated inferred data.

27. A method of treating a patient using a computational guideline of a knowledge graph, said method comprising:
inputting a patient condition and matching said patient condition to a medical condition node of said knowledge graph, said knowledge graph including a plurality of nodes representing a medical guideline;
traversing said guideline nodes from said medical condition node, wherein when a drug therapy node is reached, outputting a recommendation to prescribe a medication that is represented by a child node of said drug therapy node; and
continuing said traversing of said guideline nodes from a child node of said drug therapy node.

28. A method as recited in claim 27 when said drug therapy node is reached, said method further comprising:
traversing to a therapeutic intervention node of said knowledge graph that is linked to said medical condition node; and
outputting a recommendation for said patient to perform a drug therapy represented by said therapeutic intervention node.

29. A method as recited in claim 27 wherein when a decision node of said guideline nodes is reached, said method further comprising:
matching a diagnostic factor of said patient or an investigation result of said patient to a child node of said decision node and continuing said traversing from said child node.

30. A method as recited in claim 27, wherein when a determination node of said guideline nodes is reached, said method further comprising:
determining a result of said medical guideline; and
outputting said result of said medical guideline.
